(19) United States
(12) Patent Application Publication
(10) Pub. No.: US 2023/0312752 A1
(43) Pub. Date: Oct. 5, 2023

(54) METHODS FOR PREVENTING, REVERSING OR TREATING AN INFECTION INDUCED BY A VIRUS THAT ENTERS HOST CELLS VIA ACE2 RECEPTOR

(71) Applicant: Brown University, Providence, RI (US)

(72) Inventors: Jack A. ELIAS, Providence, RI (US); Chun Geun LEE, Woodbridge, CT (US); Suchitra KAMLE, Providence, RI (US); Bing MA, Branford, CT (US); Changmin Lee, Warwick, RI (US)

(21) Appl. No.: 18/058,167

(22) Filed: Nov. 22, 2022

Related U.S. Application Data
(60) Provisional application No. 63/282,910, filed on Nov. 24, 2021.
(60) Provisional application No. 63/282,956, filed on Nov. 24, 2021.
(60) Provisional application No. 63/301,331, filed on Jan. 20, 2022.
(60) Provisional application No. 63/301,338, filed on Jan. 20, 2022.

(51) Int. Cl. C07K 16/40; A61P 31/14

(52) U.S. Cl. CPC C07K 16/40; A61P 31/14; C07K 2317/565; C07K 2317/24

(57) ABSTRACT

Described herein are methods of preventing, reversing, and/or treating an infection induced by a virus that enters cells via the cellular receptor Angiotensin-Converting Enzyme 2 (ACE2) by administering an inhibitor of CHI3L1.

### CROSS-REFERENCE TO RELATED APPLICATIONS

This application claims benefit under 35 U.S.C. § 119(e) of U.S. Provisional Application No. 63/282,910 filed Nov. 24, 2021, U.S. Provisional Application No. 63/282,956 filed Nov. 24, 2021, U.S. Provisional Application No. 63/301,331 filed Jan. 20, 2022, and U.S. Provisional Application No. 63/301,338 filed Jan. 20, 2022, the contents of which are incorporated herein by reference in their entirety.

### FIELD OF THE INVENTION

The embodiments of the present invention relate to methods for the prevention, reversal, and/or treatment of an infection induced by a virus that enters host cells via the cellular receptor Angiotensin-Converting Enzyme 2 (ACE2). More specifically, the methods involve the administration of an inhibitor of CHI3L1 such as an anti-CHI3L1 antibody, the small molecule inhibitor kasugamycin, and/or an inhibitor of CHI3L1 phosphorylation.

### SEQUENCE LISTING

The instant application contains a Sequence Listing which has been submitted electronically in XML format and is hereby incorporated by reference in its entirety. Said XML copy, created on May 17, 2023, is named 405505-701001US_SL.xml and is 56,175 bytes in size.

### BACKGROUND OF THE INVENTION

Coronaviruses are a group of positive-stranded, enveloped RNA viruses that circulate broadly among humans, other mammals, and birds, causing respiratory, enteric, or hepatic diseases.¹ Since the turn of the century, coronaviruses have caused three major outbreaks. First, severe acute respiratory syndrome coronavirus (SARS-CoV) appeared in the Chinese Guangdong province in 2002. This outbreak had over 8000 infections worldwide, with a 10% fatality rate.², Only a decade later, the next large-scale coronavirus outbreak to arise was the Middle East respiratory syndrome coronavirus (MERS-CoV) in 2012. MERS-CoV infected fewer people than SARS-CoV but had a higher fatality rate of 36%.⁴ Research teams soon discovered other coronavirus isolates phylogenetically similar to the virulent SARS-CoV and MERS-CoV human coronavirus strains and warned about the high risk of novel coronaviruses entering the human population.^{5,6} In December 2019, a novel coronavirus, SARS-CoV-2, appeared in Wuhan, Hubei Province, in Central China, which quickly showed human-to-human transmission.⁷ As of November 2021, there have been 259M SARS-CoV-2 infections in the world, with 5.18M deaths.

A coronavirus contains four structural proteins, including spike (S), envelope (E), membrane (M), and nucleocapsid (N) proteins. Among them, S protein plays the most important roles in viral attachment, fusion and entry, and it serves as a target for development of antibodies, entry inhibitors and vaccines.^{8,9} The S protein mediates viral entry into host cells by first binding to a host receptor through the receptor-binding domain (RBD) in the S1 subunit and then fusing the viral and host membranes through the S2 subunit.^{10,11} In 2003, Angiotensin-Converting Enzyme-2 (ACE-2) was reported to be the receptor for SARS-CoV.¹² SARS-CoV and MERS-CoV RBDs recognize different receptors, with MERS-CoV recognizing dipeptidyl peptidase 4 (DPP4) as its receptor.¹³ Similar to SARS-CoV, SARS-CoV-2 also recognizes ACE2 as its host receptor binding to viral S protein.¹⁴ Therefore, the RBD can be defined as the most likely target for the development of virus attachment inhibitors, neutralizing antibodies, and vaccines.

Accordingly, there is a need for more effective therapeutic agents that inhibit the attachment of the virus to host cell receptors.

### BRIEF SUMMARY OF THE INVENTION

The present invention is based on the seminal discoveries that: (1) chitinase 3 like 1 (CHI3L1) stimulates/upregulates ACE2 and a few of the spike activating proteases (SAPs) required for the spike protein to enter the host cells; and (2) this CHI3L1 stimulation of ACE2 and the SAPs was fully reversed by a CHI3L1 inhibitor.

The embodiments of the present disclosure provide a method for preventing or treating an infection induced by a virus that enters host cells via the ACE2 cellular receptor, comprising administering a therapeutically effective amount of an inhibitor of CHI3L1 to subject at risk of, or afflicted with, the viral infection.

In some embodiments, the inhibitor of CHI3L1 is an antibody, antibody reagent, antigen-binding fragment thereof, or chimeric antigen receptor (CAR), that specifically binds a CHI3L1 polypeptide.

In some embodiments, the antibody, antibody reagent, antigen-binding portion thereof, or CAR comprises at least one complementarity determining regions (CDRs) selected from: (a) a light chain CDR1 having the amino acid sequence of SEQ ID NO: 4; (b) a light chain CDR2 having the amino acid sequence of SEQ ID NO: 5; (c) a light chain CDR3 having the amino acid sequence of SEQ ID NO: 6; (d) a heavy chain CDR1 having the amino acid sequence of SEQ ID NO: 1; (e) a heavy chain CDR2 having the amino acid sequence of SEQ ID NO: 2; and (f) a heavy chain CDR3 having the amino acid sequence of SEQ ID NO: 3.

In some embodiments, the antibody, antibody reagent, antigen-binding portion thereof, or CAR comprises a heavy chain sequence having the amino acid sequence selected from any one of SEQ ID NOS: 15-26. In some embodiments, the antibody, antibody reagent, antigen-binding portion thereof, or CAR comprises a heavy chain sequence having the amino acid sequence selected from any one of SEQ ID NOS: 27-34. In some embodiments, the antibody, antibody reagent, antigen-binding portion thereof, or CAR comprises a heavy chain sequence having the amino acid sequence selected from any of SEQ ID NOS: 15-26 and a light chain sequence having the amino acid sequence selected from any one of SEQ ID NOS: 27-34.

In some embodiments, the antibody, antibody reagent, antigen-binding portion thereof, or CAR comprises a heavy chain sequence having the amino acid sequence of SEQ ID NO: 13. In some embodiments, the antibody, antibody reagent, antigen-binding portion thereof, or CAR comprises a light chain sequence having the amino acid sequence of SEQ ID NO: 14. In some embodiments, the antibody, antibody reagent, antigen-binding portion thereof, or CAR comprises a heavy chain sequence having the amino acid sequence of SEQ ID NO: 13 and a light chain sequence having the amino acid sequence of SEQ ID NO: 14. In some embodiments, the antibody, antibody reagent, antigen-binding portion thereof, or CAR further comprises a conservative substitution relative to the heavy chain sequence or the light chain sequence, wherein the conservative substitution is in a sequence not comprised by a CDR. In some embodiments, the antibody, antibody reagent, antigen-binding portion thereof, or CAR is fully humanized except for the CDR sequences. In some embodiments, the antibody, antibody reagent, antigen-binding portion thereof, or CAR is selected from the group consisting of: an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a diabody, a multispecific antibody, a dual specific antibody, an anti-idiotypic antibody, and a bispecific antibody.

In alternative embodiments, the inhibitor of CHI3L1 is an inhibitor CHI3L1 and/or chitinase 1. In some embodiments, the inhibitor of CHI3L1 and/or chitinase 1 is kasugamycin (KSM) or a derivative, analog, or variant thereof. In some embodiments, the inhibitor of CHI3L1 and chitinase 1 is KSM.

In yet other alternative embodiments, the inhibitor of CHI3L1 phosphorylation is a CDK inhibitor. In some embodiments, the inhibitor of CHI3L1 is a CDK inhibitor. In some embodiments, the CDK inhibitor is selected from the group consisting of: a broad CDK inhibitor, a specific CDK inhibitor, and a multiple target inhibitor. In some embodiments, the CDK inhibitor has potency for at least one CDK isomer selected from the group consisting of: CDK1, CDK2, CDK3, CDK4, CDK5, CDK6, CDK7, CDK8, CDK9, and CLK. In some embodiments, the CDK inhibitor has potency for CDK1. In some embodiments, the CDK inhibitor has potency for CDK5. In some embodiments, the CDK inhibitor is selected from the group consisting of: Flavopiridol, Flavopiridol HCl, AT7519, BS-181 HCl, JNJ-7706621, Palbociclib HCl, PHA-793887, Roscovitine, SNS-032, A-674563, Milciclib, AZD5438, Dinaciclib, BMS-265246, PHA-767491, MK-8776, R547, Kenpaulione, AT7519 HCl, CGP60474, Wogonin, Purvalanol B, NU 6102, LY2835219 (abemaciclib), P276-00, Ribociclib, TG003, Palbociclib Isethionate, AMG-925, NU6027, THZI, LDC000067, ML167, SU9516, Ro-3306, CVT 313, NVP-LCQ195, Purvalanol A, NU2058, LY2857785, K03861, and Abemaciclib. In some embodiments, the CDK inhibitor is Flavopiridol or Flavopiridol HCl.

In some embodiments, the subject is also administered a therapeutically effective amount of a combination of CHI3L1 inhibitors. The combination can include at least two of: (i) an inhibitor of CHI3L1 such as an anti-CHI3L1 antibody; (ii) an inhibitor of CHI3L1 and chitinase 1; and/or (iii) an inhibitor of CHI3L1 phosphorylation. In some embodiments, the one or more CHI3L1 inhibitors can be combined with (i) remdesivir; (ii) dexamethasone; (iii) REGEN-COV-2 (Regeneron); (iv) Baricitinib (Lilly); (v) Sotrovimab (Vir); (vi) PAXLOVID™ (Pfizer); and/or molnupiravir (Merck).

In some embodiments, the subject has been exposed to another subject is afflicted with an ACE2-based viral infection and is administered a therapeutically effective amount of an inhibitor of CHI3L1 as a preventative measure. In some embodiments, the subject has tested positive for an ACE2-based viral infection in a diagnostic test and is administered a therapeutically effective amount of an inhibitor of CHI3L1 to reverse or prevent symptoms of the ACE2-based viral infection. In some embodiments, the subject displays one or more of the symptoms selected from the group consisting of: fever, chills, cough, shortness of breath, difficulty breathing, fatigue, muscle aches, body aches, headache, loss of taste or smell, sore throat, congestion, runny nose, nausea, vomiting, diarrhea, new confusion, inability to wake or stay awake, and bluish lips or face.

Other implementations are also described and recited herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For the purpose of illustration, certain embodiments of the present invention are shown in the drawings described below. It should be understood, however, that the invention is not limited to the precise arrangements, dimensions, and instruments shown. In the drawings:

FIG. 1 shows the effects of recombinant human CHI13L1 (rhCHI3L1) on the levels of mRNA encoding human Angiotensin-Converting Enzyme 2 (ACE2) and cathepsin L (CTSL) in A549 cells. Cells were incubated with rhCHI3L1 from a commercial source (R&D Inc.) and rhCHI3L1 generated at Brown University (Brown) and compared to vehicle controls. mRNA was then extracted and the levels of mRNA for ACE2 and CTSL were evaluated by real time qRT-PCR. Levels were expressed in relationship to GAPDH controls. *p<0.05; **p<0.01 (Student's t-test).

FIG. 2 shows the kinetics of the rhCHI3L1 regulation of ACE2, transmembrane protease serine 2 (TMPRSS2), and cathepsin L (CTSL) in A549 epithelial cells. A549 cells were incubated with the noted concentrations of rhCHI3L1 for the note amounts of time. The levels of mRNA encoding ACE2, TMPRSS2, and CTSL were assessed via qRT-PCR. The statistics compare cells incubated with vehicle control (nothing tested, NT) and cells incubated with 250 ng/ml of rhCHI3L1. The levels are expressed in relationship to GAPDH controls. Ns, not significant; *p<0.05; **p<0.01 (Student's t-test).

FIG. 3 shows the dose response of CHI3L1 regulation of ACE2, TMPRSS2, and CTSL in normal human small airway epithelial cells (HSAEC). HSAEC were incubated with the noted concentrations of recombinant rhCHI3L1 for 24 hours. The levels of mRNA encoding ACE2, TMPRSS2, and CTSL were assessed via qRT-PCR. The levels are expressed in relationship to GAPDH controls. *p<0.05 (Student's t-test).

FIG. 4 shows the effects of an anti-CHI3L1 monoclonal antibody, FRG, on the basal and rhCHI3L1 regulated expression of ACE2, FURIN, TMPRSS2, and CTSL in Calu-3 lung epithelial cells. Calu-3 cells were incubated with vehicle or rhCHI3L1 for 24 hours in the presence of FRG or its isotype control (isotype). Levels of mRNA encoding ACE2 (top left), FURIN (top right), TMPRSS2 (bottom left), and CTSL (bottom right) were assessed via RT-PCR. FRG modestly diminished the levels of basal ACE2 expression and potently ameliorated the ability of rhCHI3L1 to stimulate ACE2 mRNA accumulation. FRG also potently decreased the basal and rhCHI3L1 stimulated expression of TMPRSS2, CTSL and FURIN. *p<0.05; **p<0.001; ***p<0.001 (Student's t-test).

FIG. 5 shows the effects of transgenic CHI3L1 on the levels of mRNA encoding Ace2 and Ctsl in the murine lung. Lungs were obtained from wild type (WT; −) and lung targeted CHI3L1 transgenic (Tg; +) mice. mRNA was extracted, and the levels of mRNA for Ace2 and Ctsl were evaluated by real-time qRT-PCR. Levels were expressed in relationship to p-actin controls. Glyceraldehyde-3-phosphate dehydrogenase (Gapdh) was used as an internal control. *p<0.05 (Student's t-test).

FIG. 6A-B shows the immunohistochemical evaluation of Ace2 and Ctsl expression in lungs from WT and CHI3L1 Tg mice. FIG. 6A shows Ace2 expression in the lungs of WT and CHI3L1 Tg mice. FIG. 6B shows Ctsl expression in lungs from WT and CHI3L1 mice. Blue-fluorescent DAPI (4′,6-diamidino-2-phenylindole) was used for nuclei stain. Red-fluorescence (RFP) and green fluorescence (FITC)-labeled antibodies against Ace2 and Ctsl were used for detection of ACE2 and CTSL expression or accumulation in the lungs, respectively. ×40 of original magnification. Arrows in subset of panels indicate the expression of Ace2 or Ctsl in airway epithelial cells.

FIG. 7 shows the double label immunohistochemistry (IHC) comparing the accumulation of Tmprss2 and Ctsl in lungs from wild type (WT) and CHI3L1 overexpressing transgenic (Tg) mice. Tmprss2 stains green. Ctsl stains red. Co-localized enzymes stain yellow. Heightened co-localized staining of Tmprss2 and Ctsl can be seen in airway and, to a lesser degree, alveolar epithelial cells. ×40 of original magnification.

FIG. 8 shows the effects of kasugamycin (KSM) on the basal and rhCHI3L1 regulated expression of ACE2, FURIN, TMPRSS2, and CTSL in Calu-3 lung epithelial cells. Calu-3 cells were incubated with vehicle or rhCHI3L1 for 24 hours in the presence of KSM (250 ng/mL) or its vehicle control (PBS). Levels of mRNA encoding ACE2 (top left), FURIN (top right), TMPRSS2 (bottom left), and CTSL (bottom right) were assessed via RT-PCR. KSM diminished the levels of basal ACE2 expression and potently ameliorated the ability of rhCHI3L1 to stimulate ACE2 mRNA accumulation. KSM also potently decreased the basal and rhCHI3L1-stimulated expression of TMPRSS2, CTSL and FURIN. *p<0.05, **p<0.01; ***p<0.001; ****p<0.0001 (Student's t-test).

FIG. 9 shows the time course and dose response of CDK regulation of CHI3L1 phosphorylation. U87 cells that endogenously express all components of chitosome were subjected to Co-IP/Immunoblot assays after Pan-CDK inhibitor flavopiridol treatment. Flavopiridol was used at 50 nM unless otherwise indicated. Hr=hours.

FIG. 10 shows the effects of flavopiridol (Flavo) on the basal and CHI3L1 regulated expression of ACE2 and spike activating proteases (SAPs) in Calu-3 lung epithelial cells. After transfection of the cells with empty vector (pcDNA) or full length CHI3L1 cDNA, the Calu-3 cells were incubated with flavopiridol (25 nM) or its vehicle control for 24 hours. Levels of mRNA encoding ACE2 (top left), TMPRSS2 (top right), CTSL (bottom left), and FURIN (bottom right) were assessed via RT-PCR. *p<0.05; **p<0.01 (Student's t-test).

FIG. 11A-B shows that CHI3L1 stimulates cellular integration of S proteins and FRG abrogates the CHI3L1 effect. Calu-3 cells were incubated with vehicle (rCHI3L1(−)) or the noted concentrations of rCHI3L1 for 24 hours and then transfected with a pseudovirus containing the S protein (PS; D614 and G164 variants) from SARS-CoV-2 (SC2) and a GFP expression construct. The transfected cells were incubated for additional 24 hours and evaluated using fluorescent microscopy. FIG. 11A shows the quantification of mean fluorescent intensity (MFI), as can be seen in the dot plot on the right. In FIG. 11B, Calu-3 cells were incubated with rCHI3L1 (250 ng/mL) or vehicle (PBS) for 24 hours in the presence or absence of an antibody against CHI3L1 (the FRG antibody) or control antibody (IgG). The Calu-3 cells were infected with spike protein (S)-containing pseudovirus (PS-S; D614 and G614 variants) expressing GFP and GFP expression was evaluated by flow cytometry. ***P<0.001 (One-Way ANOVA with post hoc Dunnett's multiple comparison test).

FIG. 12 shows that CHI3L1 stimulates cellular integration of Spike proteins of SARS-Cov2 and CHI3L1 inhibitors abrogate the CHI3L1-stimulated pseudoviral infection effect of various variant forms of S proteins. Calu-3 cells were incubated with either the vehicle (PBS), a control antibody (IgG), FRG (an anti-CHI3L1 antibody), or Kasugamycin (KSM) with or without stimulation of recombinant CHI3L1 (rCHI3L1; 250 ng/mL) for 24 hours. They were then transfected with a pseudovirus (PS) containing the various mutations of S protein (D614G, E484K, United Kingdom (UK strain), South African (SA). Brazilian (BZ) from SARS-CoV-2 (SC2) and a GFP expression construct. The transfected cells were incubated for additional 48 hours and then evaluated by FACS analysis. The numbers shown in each subpanel represent % of GFP positive cells.

FIG. 13A-E shows that CHI3L1 stimulates cellular integration of pseudovirus with the ancestral G614 S protein (FIG. 13A), and mutated S proteins with alpha (FIG. 13B), beta (FIG. 13C), gamma (FIG. 13D), or delta (FIG. 13E) variants. The numbers shown in each subpanel represent % of GFP positive cells.

FIG. 14A-E shows that CHI3L1 stimulates cellular integration of Spike proteins of SARS-Cov2 and FRG, an anti-CHI3L1 monoclonal antibody, abrogates the CHI3L1-stimulated pseudoviral infection effect of various variant forms of S proteins. Calu-3 cells were incubated with either the vehicle (PBS), a control antibody (IgG), or FRG, with or without stimulation of recombinant CHI3L1 (rCHI3L1; 250 ng/mL) for 24 hours. Cells were then transfected with a pseudovirus (PS) containing the ancestral G614 (WT) S protein (FIG. 14A) or one of various mutations of the S protein from SARS-CoV-2 (SC2) and a GFP expression construct. Variants tested included the United Kingdom (alpha) variant (FIG. 14B), the South African (beta) variant (FIG. 14C), the Brazilian (gamma) variant (FIG. 14D), or the Indian (delta) variant (FIG. 14E). The transfected cells were incubated for additional 60 hours and then evaluated by FACS analysis. The numbers shown in each subpanel represent % of GFP positive cells.

FIG. 15A-E shows that CHI3L1 stimulates cellular integration of Spike proteins of SARS-Cov2 and kasugamycin (KSM), an inhibitor of CHI3L1 and chitinase 1, abrogates the CHI3L1-stimulated pseudoviral infection effect of various variant forms of S proteins. Calu-3 cells were incubated with either the vehicle (PBS), a control antibody (IgG), or KSM, with or without stimulation of recombinant CHI3L1 (rCHI3L1; 250 ng/mL) for 24 hours. Cells were then transfected with a pseudovirus (PS) containing the ancestral G614 (WT) S protein (FIG. 15A) or one of various mutations of the S protein from SARS-CoV-2 (SC2) and a GFP expression construct. Variants tested included the United Kingdom (alpha) variant (FIG. 15B), the South African (beta) variant (FIG. 15C), the Brazilian (gamma) variant (FIG. 15D), or the Indian (delta) variant (FIG. 15E). The transfected cells were incubated for additional 60 hours and then evaluated by FACS analysis. The numbers shown in each subpanel represent % of GFP positive cells.

FIG. 16 shows the CHI3L1 stimulation of pseudovirus uptake. Calu-3 cells were incubated with recombinant human (rh) CHI3L1 (CHI3L1, 250 ng/mL) or vehicle (PBS) control for 48 hours. Pseudoviruses (PS) that contain S proteins with G614, alpha, beta, gamma or delta mutations were added and GFP was quantitated by FACS. The % of GFP-positive cells was evaluated by flow cytometry. The noted values are representative of a minimum of three similar evaluations.

FIG. 17A-E shows the effects of FRG on G614, alpha, beta, gamma and delta pseudovirus infection. Calu-3 cells were incubated with rhCHI3L1 (CHI3L1, 250 ng/mL) or vehicle control for 48 hours in the presence of anti-CHI3L1 (FRG) or its isotype control (IgG). Pseudoviruses (PS) that contain S proteins with the G614, alpha, beta, gamma or delta mutations were added and GFP was quantitated by FACS. The % of GFP-positive cells was evaluated by flow cytometry. The noted values are representative of a minimum of 3 similar evaluations.

FIG. 18A-C shows the immunocytochemical evaluation of delta pseudovirus infection of Calu-3 cells. Calu-3 cells were incubated in the presence and or absence of rhCHI3L1 (CHI3L1) in the presence of FRG or its isotype control. FIG. 18A: Pseudoviruses with delta S proteins (PS-d) were added and ACE2 and GFP viral infection were evaluated using double labeled immunocytochemistry (ICC). DAPI (blue) was used to evaluate nuclei, red label was used to evaluate ACE2 and the pseudoviruses contained GFP. FIG. 18B-C: The quantification of ACE2 can be seen in panel (FIG. 18B) and the quantification of GFP is illustrated in panel (FIG. 18C). These evaluations were done using fluorescent microscopy (×20 of original magnification). In these quantifications, five randomly selected fields were evaluated. The values in FIG. 18B-C are the mean±SEM of the noted 5 evaluations. **P<0.01; ***P<0.001, ****P<0.0001; ns=not significant (One way ANOVA with multiple comparisons). Scale bar: 10 μm (applies to all subpanels in FIG. 18A)

FIG. 19 shows the Kasugamycin inhibition of CHI3L1-induced signaling. Calu-3 cells were stimulated with rhCHI3L1 (250 ng/mL) or its vehicle control for 6-12 hours in the presence of Kasugamycin (250 ng/mL) and vehicle control (PBS). Western blotting was then employed to evaluate the levels of activated (phosphorylated, p) and total (t) ERK and AKT. The noted figure is representative of a minimum of three similar experiments.

FIG. 20 shows the effects of Kasugamycin on alpha, beta, gamma and delta pseudovirus infection. Calu-3 cells were incubated with rhCHI3L1 (250 ng/mL) or vehicle control for 48 hours in the presence of kasugamycin or its vehicle control. Pseudoviruses that contain S proteins with the G614, alpha, beta, gamma or delta mutations were added and GFP was quantitated by FACS analysis. The % of GFP-positive cells was evaluated by flow cytometry. The noted values are representative of a minimum of three similar evaluations.

FIG. 21A-C shows the immunocytotochemical evaluation of delta pseudovirus infection of Calu-3 cells. Calu-3 cells were incubated in the presence and or absence of CHI3L1 (250 ng/mL) in the presence or of Kasugamycin (250 ng/mL) or its vehicle control. FIG. 21A: Pseudoviruses with delta S proteins were added and ACE2 and GFP viral infection were evaluated using double labeled immunocytochemistry (ICC). DAPI (blue) was used to evaluate nuclei, red label was used to evaluate ACE2 and the pseudoviruses contained GFF. FIG. 21B-C: The quantification of ACE2 can be seen in panel (FIG. 21B) and the quantification of GFP is illustrated in panel (FIG. 21C). These evaluations were done using fluorescent microscopy (×20 of original magnification). In these quantifications, five randomly selected fields were evaluated. The values in FIG. 21B-C are the mean±SEM of the noted five evaluations. *P<0.05, **P<0.01; ***P<0.001; ns=not significant (One way ANOVA with multiple comparisons). Scale bar: 10 μm (applies to all subpanels in FIG. 21A).

FIG. 22 shows the effects of FRG and Kasugamycin on omicron pseudovirus infection. Calu-3 cells were incubated with rhCHI3L1 (CHI3L1, 250 ng/mL) or vehicle control for 48 hours in the presence or absence of anti-CHI3L1 (FRG) or its isotype control (IgG) or kasugamycin or vehicle control. Pseudoviruses that contain S proteins with the omicron mutations (PS-o) were added and GFP was quantitated by FACS. The % of GFP-positive cells was evaluated by flow cytometry. The noted values are representative of a minimum of three similar evaluations.

### DETAILED DESCRIPTION OF THE INVENTION

It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

Singular forms: As used in this specification and the appended claims, the singular forms “a,” “an” and “the” include plural referents unless the content clearly dictates otherwise. For example, reference to “a cell” includes a combination of two or more cells, and the like.

Alternative forms: As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Exempli gratia: The abbreviation, “e.g.” is derived from the Latin exempli gratia and is used herein to indicate a non-limiting example. Thus, the abbreviation “e.g.” is synonymous with the term “for example.”

Approximately or about: As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

Comprising: As used herein, the term “comprising” means that other elements can also be present in addition to the defined elements presented. The use of “comprising” indicates inclusion rather than limitation.

Consisting of: The term “consisting of” refers to compositions, methods, and respective components thereof as described herein, which are exclusive of any element not recited in that description of the embodiment.

Consisting essentially of: As used herein the term “consisting essentially of” refers to those elements required for a given embodiment. The term permits the presence of additional elements that do not materially affect the basic and novel or functional characteristic(s) of that embodiment of the invention.

Statistically significant: The term “statistically significant” or “significantly” refers to statistical significance and generally means a two standard deviation (2SD) or greater difference.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition (e.g., one or more symptoms or features of a Covid-19 infection).

Therapeutic-effective amount: As used herein, the phrase “therapeutically-effective amount”, “effective amount” or “effective dose” refers to an amount that provides a therapeutic or aesthetic benefit in the treatment, prevention, or management of a Covid-19 infection, e.g., an amount that provides a statistically significant decrease in at least one symptom, sign, or marker of a Covid-19 infection. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic agents.

Treatment: As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

Administration: As used herein, the term “administering,” refers to the placement of a Covid-19 therapeutic agent, as disclosed herein, into a subject by a method or route which results in at least partial delivery of the agent at a desired site. Pharmaceutical compositions comprising the compounds disclosed herein can be administered by any appropriate route which results in an effective treatment in the subject.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks, or more.

The administration of the compositions contemplated herein may be carried out in any convenient manner, including by aerosol inhalation, injection, ingestion, transfusion, implantation or transplantation. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

Covid-19 subject: A subject that has a Covid-19 infection is a subject having a measurable level of SARS-CoV-2, the virus that causes Covid-19. Viral tests check samples from the subject's respiratory system, such as a swab from the inside of the subject's nose or throat. Some tests are point-of-care tests, meaning results may be available at the testing site in less than an hour. Other tests must be sent to a laboratory for analysis, a process that takes 1-2 days or longer.

Symptoms of Covid-19 may appear 2-14 days after exposure to the virus and include, but are not limited to, fever or chills, cough, shortness of breath or difficulty breathing, fatigue, muscle or body aches, headache, loss of taste or smell, sore throat, congestion or runny nose, nausea or vomiting, diarrhea. Emergency warning signs for Covid-19 include, but are not limited to, trouble breathing, persistent pain or pressure in the chest, new confusion, inability to wake or stay awake, or bluish lips or face.

Decrease: The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

Increase: The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

Polypeptide: As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

Variants: In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

Nucleic acid: As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

Expression vector: As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

Isolated: The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

Engineered: As used herein, “engineered” refers to the aspect of having been manipulated by the hand of man. For example, an antibody, antibody reagent, antigen-binding portion thereof, or CAR antibody is considered to be “engineered” when the sequence of the antibody, antibody reagent, antigen-binding portion thereof, or CAR antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

Epitope: As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. A single domain antibody (sdAb), also known as a nanobody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).¹⁵ The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

Antibody: As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (sdAb), a diabody, a multispecific antibody, a dual specific antibody, an anti-idiotypic antibody, an antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

Complementarity determining regions: As used herein, the term “CDR” refers to the complementarity determining regions within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems. The system described by Kabat et al. (1987) and (1991) not only provides an unambiguous residue numbering system applicable to any variable region of an antibody, but also provides precise residue boundaries defining the three CDRs. These CDRs may be referred to as Kabat CDRs. Other boundaries defining CDRs overlapping with the Kabat CDRs have been described by Padlan et al. (1995),¹⁶ MacCallum et al. (1996),¹⁷ and Chothia et al. (1987)¹⁸ and (1989).¹⁹ Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat defined CDRs.

Antigen-binding portion: The term “antigen-binding portion” of an antibody refers to one or more portions of an antibody as described herein, said portions) still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a sdAb portion consisting of a VH domain or of VH, CH1, CH2, DH3, or VH, CH2, CH3 (sdAbs, or single domain antibodies, comprising only V_{L} domains have also been shown to specifically bind to target epitopes). Although the two domains of the Fv portion, namely VL and VH, are encoded by separate genes, they may further be linked to one another using a synthetic linker, e.g., a poly-G4S amino acid sequence (‘G4S’ disclosed as SEQ ID NO: 29 in U.S. Pat. No. 10,253,111),²⁰ (“G4S” disclosed as SEQ ID NO: 47) and recombinant methods, making it possible to prepare them as a single protein chain in which the VL and VH regions combine in order to form monovalent molecules (known as single chain Fv (ScFv)). The term “antigen-binding portion” of an antibody is also intended to comprise such single chain antibodies. Other forms of single chain antibodies such as “diabodies” are likewise included here. Diabodies are bivalent antibodies in which VH and VL domains are expressed on a single polypeptide chain but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

Antigen reagent: As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (sdAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody, antigen-binding portion thereof, or CAR as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cysteine residue, a marker peptide and a C-terminal polyhistidinyl, e.g., hexahistidinyl tag (‘hexahistidinyl tag’ disclosed as SEQ ID NO: 30 in U.S. Pat. No. 10,253,111) (“Hexahistidinyl” disclosed as SEQ ID NO: 48) in order to produce bivalent and biotinylated scFv molecules.

In some embodiments, the antibody, antibody reagent, antigen-binding portion thereof, or CAR described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide. In some embodiments, the CAR comprises an extracellular domain that binds CHI3L1, wherein the extracellular domain comprises a humanized or chimeric antibody or antigen-binding portion thereof.

Human antibody: The term “human antibody” refers to antibodies whose variable and constant regions correspond to or are derived from immunoglobulin sequences of the human germ line, as described, for example, by Kabat et al. (1991)²¹ However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line. See, Kabat, et al. (1991). According to particular embodiments, however, such recombinant human antibodies are subjected to in vitro mutagenesis (or to a somatic in vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089;²² 6,824,989;²³ and 6,835,823.²⁴ These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

In some embodiments, the antibody, antibody reagent, antigen-binding portion thereof, and/or CAR as described herein can be a variant of a sequence described herein, e.g., a conservative substitution variant of an antibody polypeptide. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or portion thereof that retains activity, e.g., antigen-specific binding activity for the relevant target polypeptide, e.g., CHI3L1. A wide variety of PCR-based site-specific mutagenesis approaches are also known in the art and can be applied by the ordinarily skilled artisan.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin). The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

Chimeric antibody: In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Humanized antibody: Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody, antigen-binding portion thereof, or CAR as described herein. Such functional activities include anti-CHI3L1 activity. Additionally, a polypeptide having functional activity means the polypeptide exhibits activity similar, but not necessarily identical to, an activity of a reference antibody, antigen-binding portion thereof, or CAR as described herein, including mature forms, as measured in a particular assay, such as, for example, a biological assay, with or without dose dependency. In the case where dose dependency does exist, it need not be identical to that of the reference antibody, antigen-binding portion thereof, or CAR, but rather substantially similar to the dose-dependence in a given activity as compared to the reference antibody, antigen-binding portion thereof, or CAR as described herein (i.e., the candidate polypeptide will exhibit greater activity, or not more than about 25-fold less, about 10-fold less, or about 3-fold less activity relative to the antibodies, antigen-binding portions, and/or CARs described herein).

In some embodiments, the antibody reagents (e.g., antibodies or CARs) described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

In some embodiments, the antibody, antibody reagent, antigen-binding portion thereof, and/or CAR is an isolated polypeptide. In some embodiments, the antibody, antibody reagent, antigen-binding portion thereof, and/or CAR is a purified polypeptide. In some embodiments, the antibody, antibody reagent, antigen-binding portion thereof, and/or CAR is an engineered polypeptide.

Avidity: “Avidity” is the measure of the strength of binding between an antigen-binding molecule (such as an antibody or antigen-binding portion thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D} of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (K_{A}) of 10⁵ to 10¹² liter/moles or more, such as 10⁷ to 10¹² liter/moles or 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

Accordingly, as used herein, “selectively binds” or “specifically binds” refers to the ability of a peptide (e.g., an antibody, CAR, bispecific antibody or portion thereof) described herein to bind to a target, such as an antigen present on the cell-surface of a cell, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody, antigen-binding portion thereof, CAR or bispecific antibody is said to specifically bind an antigen when it preferentially recognizes its target antigen in a complex mixture of proteins and/or macromolecules.

In some embodiments, an antibody, antigen-binding portion thereof, or CAR, as described herein, binds to CHI3L1 with a dissociation constant (K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, an antibody, antigen-binding portion thereof, or CAR, as described herein, binds to CHI3L1 with a dissociation constant (K_{D}) of from about 10⁻⁵ M to 10⁻⁶ M. In some embodiments, an antibody, antigen-binding portion thereof, or CAR, as described herein, binds to CHI3L1 with a dissociation constant (K_{D}) of from about 10⁻⁶ M to 10⁻⁷ M. In some embodiments, an antibody, antigen-binding portion thereof, or CAR, as described herein, binds to CHI3L1 with a dissociation constant (K_{D}) of from about 10⁻⁷ M to 10⁻⁸ M. In some embodiments, an antibody, antigen-binding portion thereof, or CAR, as described herein, binds to CHI3L1 with a dissociation constant (K_{D}) of from about 10⁻⁸ M to 10⁻⁹ M. In some embodiments, an antibody, antigen-binding portion thereof, or CAR, as described herein, binds to CHI3L1 with a dissociation constant (K_{D}) of from about 10⁻⁹ M to 10⁻¹⁰ M. In some embodiments, an antibody, antigen-binding portion thereof, or CAR, as described herein, binds to CHI3L1 with a dissociation constant (K_{D}) of from about 10⁻¹⁰ M to 10⁻¹¹ M. In some embodiments, an antibody, antigen-binding portion thereof, or CAR, as described herein, binds to CHI3L1 with a dissociation constant (K_{D}) of from about 10⁻¹¹ M to 10⁻¹² M. In some embodiments, an antibody, antigen-binding portion thereof, or CAR, as described herein, binds to CHI3L1 with a dissociation constant (K_{D}) of less than 10⁻¹² M.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in THE MERCK MANUAL OF DIAGNOSIS AND THERAPY, 19th (2011);²⁵ THE ENCYCLOPEDIA OF MOLECULAR CELL BIOLOGY AND MOLECULAR MEDICINE, (1999-2012);²⁶ MOLECULAR BIOLOGY AND BIOTECHNOLOGY: A COMPREHENSIVE DESK REFERENCE, (1995);²⁷ IMMUNOLOGY (2006);²⁸ JANEWAY'S IMMUNOBIOLOGY (2014);²⁹ LEWIN'S GENES XI (2014);³⁰ MOLECULAR CLONING: A LABORATORY MANUAL, 4^{th} ed. (2012);³¹ BASIC METHODS IN MOLECULAR BIOLOGY (2012);³² LABORATORY METHODS IN ENZYMOLOGY: DNA (2013);³³ CURRENT PROTOCOLS IN MOLECULAR BIOLOGY (CPMB) (2014);³⁴ CURRENT PROTOCOLS IN PROTEIN SCIENCE (CPPS) (2005);³⁵ and CURRENT PROTOCOLS IN IMMUNOLOGY (CPI) (2003).³⁶

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### COVID-19

We are presently in the midst of a virus induced pandemic that has changed virtually everything in our day-to-day world. The resulting disease is called Covid-19 and is caused by a coronavirus called SARS-Cov-2. The virus is known to enter cells via a cellular receptor called ACE2. The viral spike or “S” protein that sticks out of the virus binds to ACE2. To get inside of the cell, the spike protein has to be modified by enzymes called spike activating proteases (SAPs) including cathepsin L and transmembrane protease, serine 2 (TMPRSS2). The binding of the virus's phosphorylated spike protein and ACE2 allows the virus to enter and do what it does in the cell. Once in the cell, the virus replicates and is subsequently released by the cell to infect other cells. In the process, it causes cell death and tissue injury, inflammation and eventually pulmonary fibrosis (scarring). When severe, massive lung injury leads to ARDS (adult respiratory distress syndrome) and, in many cases, respiratory failure and death. Unfortunately, the mechanisms of these events are poorly understood.

There are a number of interesting aspects of the epidemiology, clinical manifestations and natural history of the virus and Covid-19. They include:
(a) its striking proclivity to infect the elderly and people with comorbid diseases such as hypertension, diabetes, and obesity;
(b) the recent appreciation that an exaggerated injury, inflammation and cytokine response may contribute to the morbidity and mortality of the disease in many cases; and
(c) the recent appreciation that many of the people that develop ARDS end up chronically dependent on a ventilator with pulmonary fibrosis.
   Importantly, the mechanisms that explain these features of Covid-19 have not been defined.

We did some preliminary experiments to see if the gene family called the 18 glycosyl hydrolases has any role in Covid-19. We have found that one of the prototypes of this gene family called chitinase 3 like 1 (CHI3L1) stimulates ACE2 and a few of the SAPs. The SAP stimulation was particularly striking for cathepsin L and TMPRSS2. These data suggested that this CHI3L1-ACE2 pathway may be a major contributor to the pathogenesis of Covid-19 that can explain many of the unique features described above. Specifically:
(a) Interestingly, CHI3L1 is known to increase with aging and in the comorbid diseases mentioned above;
(b) CHI3L1 is known to drive tissue fibrosis in the lung;³⁷ and
(c) CHI3L1 is known to exist in an activated (phosphorylated) and a non-phosphorylated form with phosphorylation at specific sites being essential in the mediation of its effector functions.

With respect to viral infections, type 1 immune responses are effective antiviral responses whereas type 2 immune responses are not. T helper type 1 (Th1) lymphocytes secrete interleukin (IL)-2, interferon-γ, and lymphotoxin-α and stimulate type 1 immunity, which is characterized by intense phagocytic activity. Conversely, Th2 cells secrete IL-4, IL-5, IL-9, IL-10, and IL-13 and stimulate type 2 immunity, which is characterized by high antibody titers. Type 1 and type 2 immunity are not strictly synonymous with cell-mediated and humoral immunity, because Th1 cells also stimulate moderate levels of antibody production, whereas Th2 cells actively suppress phagocytosis. For most infections, save those caused by large eukaryotic pathogens, type 1 immunity is protective, whereas type 2 responses assist with the resolution of cell-mediated inflammation. It is important to note that CHI3L1 fosters type 2 immune responses and interventions that block CHI3L1 foster type 1 antiviral immune responses.

These above-described findings raised the exciting hypothesis that the increase in CHI3L1 with aging and with comorbid disease is responsible for the marked proclivity of the disease for the elderly or people with other diseases. It also raised the exciting idea that the CHI3L1 inhibitors might be useful in the prevention or treatment of Covid-19. Lastly, the findings suggested that an intervention that controls the ACE2 and SAPs such as cathepsin L and TMPRSS2, and fibrosis might be particularly useful in Covid-19.

### SARS-CoV-2 and Mutation Variants

Covid-19 is caused by a highly transmissible novel coronavirus, severe acute respiratory syndrome coronavirus 2 (SARS-CoV-2). First reported in Wuhan, China, on Dec. 31, 2019, this new virus presents particular dangers as there is no known pre-immunity, no vaccine, and no specific treatment.

The virus is contagious, and everyone is presumed to be susceptible. By March 2020, Covid-19 moved rapidly throughout Europe and the US. Older adults and people who have underlying medical conditions (e.g., heart or lung disease, diabetes, a BMI of 30 or higher) seem to be at higher risk for developing more serious complications from Covid-19 illness.

All viruses, including SARS-CoV-2, change over time through mutation. Most changes have little to no impact on the virus' properties. However, some changes do affect the virus's properties, such as how easily it spreads, the associated disease severity, or the performance of vaccines, therapeutic medicines, diagnostic tools, or other public health and social measures. Indeed, as SARS-CoV-2 continues to spread and cause diseases, emerging variants of the virus are being identified around the globe. The persisting challenges of SARS-CoV-2 to the international public health system have elicited concerns among scientists, drug and vaccine developers, and the general population.

For easier and more practical discussion of the variants, the World Health Organization (WHO) has designated some variants “Variants of Concerns (VOCs)” or “Variants of Interests (VOIs)” because of their ability to significantly change the virus' properties. Recently, WHO has renamed the dominantly circulating variants by Greek alphabets, i.e., alpha (α) for B.1.1.7 (U.K. variant), beta (β) for B.1.351 (South Africa), gamma (γ) for P.1 (Brazil), delta (γ) for B.1.617.2 (India), etc. Since the SARS-CoV-2 delta outbreak in India in April 2021, the highly contagious delta variant has rapidly spread all over the world and displaced alpha to be the most prevalent variant. Another variant lambda (A) for C.37 sparked headlines the summer of 2021 after the WHO noted its rapid spread in South American countries, including Peru, Ecuador, Argentina and Brazil. The WHO reported that “lambda has been associated with substantive rates of community transmission in multiple countries, with rising prevalence over time concurrent with increased COVID-19 incidence” and that more investigations would be carried out into the variant.

A more recent variant, AY.4.2, also known as “delta-plus,” is due to a mutation of the SARS-CoV-2 delta variant. The impact of this mutation is not quite clear yet. AY.4.2 has been followed by the World Health Organization (WHO) since July 2021 and is present now in dozens of countries. As of November 2021, most of the cases have occurred in the U.K. but even there, it's only present in about 6% of the cases. In the United States, it's been present in less than 1% of cases. The AY.4.2 variant has three mutations, with two of them being on the spike protein.

A more detailed description is provided below for some of the variants:

### D614G Variant

The spike aspartic acid-614 to glycine (D614G) substitution is prevalent in global SARS-CoV-2 strains. SARS-CoV-2 D614G variant shows enhanced infectivity in immortalized cell lines and replication fitness in upper human respiratory epithelia compared with the ancestral WT virus.^{38,39}

### E484K Variant

The E484K mutation is not a new variant in itself, it's a mutation which occurs in different variants and has already been found in the South African (B.1.351) and Brazilian (B.1.1.28) variants. The mutation is in the spike protein and appears to have an impact on the body's immune response and, possibly, vaccine efficacy, prompting fears the virus is evolving further and could become resistant to vaccines.⁴⁰

### United Kingdom (UK) Variant

A new SARS-CoV-2 variant (named B.1.1.7) was identified from genomic sequencing of samples from patients with covid-19 in the southeast of England in early October 2020. In December 2020, Public Health England identified this virus as a variant of concern.⁴¹ It is estimated to be 40%-80% (with most estimates occupying the middle to higher end of this range) more transmissible than the wild-type SARS-CoV-2. This increase is thought to be at least partly because of one or more mutations in the virus's spike protein. The variant is also notable for having more mutations than normally observed.⁴² The spike mutations in the B.1.1.7 variant (B.1.7: BPS BIOSCIENCE #78112-1) are:
Deletions of H69, V70, and Y144; N501Y, A570D, D614G, P681H, T716I, S982A, D1118H.

### South African (SA) Variant

On 18 Dec. 2020, the 501.V2 variant, also known as 501.V2, 20H/501Y.V2 (formerly 20C/501Y.V2), VOC-20DEC-02 (formerly VOC-202012/02), or lineage B.1.351,⁴³ was first detected in South Africa and reported by the country's health department.⁴⁴ Researchers and officials reported that the prevalence of the variant was higher among young people with no underlying health conditions, and by comparison with other variants it is more frequently resulting in serious illness in those cases.⁴⁵ The spike mutations in the B.1.351 variant (B.1.351: BPS BIOSCIENCE #78142-1) are:
L18F, D80A, D215G, R246I, K417N, E484K, N501Y, D614G, A701V.

### Brazilian (BZ) Variant

Lineage P.1, also known as 20J/501Y.V3, Variant of Concern 202101/02 (VOC-202101/02) or colloquially known as the Brazil(ian) variant, was first detected by the National Institute of Infectious Diseases (NIID), Japan, on 6 Jan. 2021 in four people who had arrived in Tokyo having visited Amazonas, Brazil, four days earlier.^{46,47} It was subsequently declared to be in circulation in Brazil.⁴⁸ This variant has 17 amino acid changes, ten of which are in its spike protein, including these three designated to be of particular concern: N501Y, E484K and K417T.⁴⁹ The spike mutations in the P.1 variant (P.1: BPS BIOSCIENCE #78144-1) are:
L18F, T20N, P26S, D138Y, R190S, K417T, E484K, N501Y, D614G, H655Y, T1027I.

### Chintinase-3-Like Protein 1 (CH3L1) Inhibitors

The GH18 proteins are members of an ancient gene family that exists in species as diverse as bacteria, plants and man. This gene family contains true chitinases (Cs) which degrade chitin polysaccharides and chitinase-like proteins (CLPs) which bind to but do not degrade chitin. Chitinase 3-like-1 (CHI3L1 or Chi3l1, also called YKL-40 in man and BRP-39 in mice), the prototypic CLP, was discovered in cancer cells and is now known to be expressed by a variety of cells including macrophages, epithelial cells and smooth muscle cells and is stimulated by a number of mediators including IL-13, IL-6, IL-1β, TGF-β1 and IFN-γ. In keeping with these diverse sources and stimuli, elevated levels of CHI3L1/YKL-40 have been noted in a variety of diseases characterized by inflammation and remodeling and a variety of malignancies.

The present invention provides a method for the treatment of a Covid-19 infection with the administration of one or more inhibitor of CHI3L1. The sequences of CHI3L1 expression products are known for a number of species, e.g., human CHI3L1 (NCBI Gene ID No: 1116) mRNA (NCBI Ref Seq: NM_001276.1 and NCBI Ref Seq: NM_001276.2) and polypeptide (NCBI Ref Seq: NP_001267.1 and NCBI Ref Seq: NP_001267.2).

### Anti-CHI3L1 Antibody

In one aspect of any of the embodiments described herein, the CHI3L1 inhibitor is an anti-CHI3L1 antibody, antibody reagent, antigen-binding fragment thereof, or chimeric antigen receptor (CAR), that specifically binds a CHI3L1 polypeptide, said antibody reagent, antigen-binding portion thereof, or CAR comprising at least one heavy or light chain complementarity determining region (CDR) selected from the group consisting of: (a) a light chain CDR1 having the amino acid sequence of SEQ ID NO: 4; (b) a light chain CDR2 having the amino acid sequence of SEQ ID NO: 5; (c) a light chain CDR3 having the amino acid sequence of SEQ ID NO: 6; (d) a heavy chain CDR1 having the amino acid sequence of SEQ ID NO: 1; (e) a heavy chain CDR2 having the amino acid sequence of SEQ ID NO: 2; and (f) a heavy chain CDR3 having the amino acid sequence of SEQ ID NO: 3; or a conservative substitution variant of one or more of (a)-(f).

In some of the embodiments, the CHI3L1 antigen-binding portion of the antibodies of the present invention include one or more of the heavy chain CDRs having the amino acid sequences of SEQ ID NOs: 1-3 and/or one or more of the light chain CDRs having the amino acid sequences of SEQ ID NOs: 4-6 disclosed in U.S. Pat. No. 10,253,111 and reproduced below in Table 1.

TABLE 1
Sequences of variable complementarity determining regions
(CDRs) of the FRG antibody
Heavy Chain CDRs

| | | |
|---|---|---|
| CDR1 | GYTFTNYG | SEQ ID NO: 1 |
| (DNA) | (GGGTATACCTTCACAAACTATGGA) | SEQ ID NO: 7 |
| CDR2 | INTYTGEP | SEQ ID NO: 2 |
| (DNA) | (ATAAATACCTACACTGGAGAGCCA) | SEQ ID NO: 8 |
| CDR3 | ARLGYGKFYVMDY | SEQ ID NO: 3 |
| (DNA) | (GCAAGATTGGGATATGGTAAATTCTATGTTATGGACTAC) | SEQ ID NO: 9 |

Light Chain CDRs

| | | |
|---|---|---|
| CDR1 | QSLVHSNGNTY | SEQ ID NO: 4 |
| (DNA) | (CAGAGCCTTGTACACAGTAATGGAAACACCTAT) | SEQ ID NO: 10 |
| CDR2 | KVS | |
| (DNA) | (AAAGTTTCC) | |
| CDR3 | SQSTHVTWT | SEQ ID NO: 6 |
| (DNA) | (TCTCAAAGTACACATGTTACGTGGACG) | SEQ ID NO: 12 |

In some embodiments of any of the aspects, the antibody, antibody reagent, antigen-binding portion thereof, or CAR comprises heavy chain CDRs having the amino acid sequences of SEQ ID NOs: 1-3 or a conservative substitution variant of such amino acid sequence. In some embodiments of any of the aspects, the antibody, antibody reagent, antigen-binding portion thereof, or CAR comprises light chain CDRs having the amino acid sequences of SEQ ID NOs: 4-6 or a conservative substitution variant of such amino acid sequence. In some embodiments of any of the aspects, the antibody, antibody reagent, antigen-binding portion thereof, or CAR comprises light chain CDRs having the amino acid sequences of SEQ ID NOs: 4-6 and heavy chain CDRs having the amino acid sequences of SEQ ID NOs: 1-3 or a conservative substitution variant of such amino acid sequence.

In one aspect of any of the embodiments, described herein is an antibody, antibody reagent, antigen-binding portion thereof, or CAR that specifically binds an CHI3L1 polypeptide, and can compete for binding of CHI3L1 with an antibody comprising light chain CDRs having the amino acid sequences of SEQ ID NOs: 4-6 and heavy chain CDRs having the amino acid sequences of SEQ ID NOs: 1-3.

In some of the embodiments, the backbone of an anti-human CHI3L1 antibody comprises a conservative substitution relative to the heavy chain sequence having the amino acid sequence of SEQ ID NO: 36 or the light chain sequence having the amino acid sequence of SED ID NO: 38 disclosed in U.S. Pat. No. 10,253,111, wherein the conservative substitution is in a sequence not comprised by a CDR. In an alternative embodiment, the backbone of an anti-human CHI3L1 antibody comprises the heavy chain sequence of the FRG antibody having the amino sequence of SEQ ID NO: 36 or the light chain sequence of the FRG antibody having the amino acid sequence of SED ID NO: 38 disclosed in U.S. Pat. No. 10,253,111, both of which are provided below as SED ID NO: 13 and SED ID NO: 14, respectively.

TABLE 2
FRG Sequences
FRG Heavy Chain Sequence
QIQLVQSGPELKKPGETVKISCKASGYTFTNYGMNWVKQAPGKGLKWMGW
INTYTGEPTYADDFKGRFAFSLETSASTAYLQINNLRNEDMSTYFCARLG
YGKFYVMDYWGQGTSVTVSS (SEQ ID NO: 13)
FRG Light Chain Sequence
DVVMTQTPLSLPVSLGDQASISCRSSQSLVHSNGNTYLHWYLQKPGQSPK
LLIYKVSNRFSGVPDRFSGSGSGTDFTLKISRVEAEDLGVYFCSQSTHVT
WTFGGGTKLEIK (SEQ ID NO: 14)

In other alternative embodiments, the CHI3L1 antigen-binding portion of the antibodies of the present invention include one or more of the heavy chain CDRs having the amino acid sequences of SEQ ID NOs: 15-26 and/or one or more of the light chain CDRs having the amino acid sequences of SEQ ID NOs: 27-34 disclosed in Table 3. See, e.g., International Published Application WO 2019/060675.⁵⁰

TABLE 3
Additional Anti-CHI3L1 Antibody Sequences
Heavy Chain CDRs
QIQLVQSGSELKKPGASVKISCKASGYTFTNYGMNWVRQAPGQGLEWMGWINTYTGEPTYADDFKGRFVFS
LDTSVSTAYLQISSLKAEDTSVYFCARLGYGKFYVMDYWGQGTSVTVSS (SEQ ID NO: 15)
QIQLVQSGSELKKPGASVKISCKASGYTFTNYGMNWVRQAPGQGLEWMGWINTYTGEPTYAQGFTGRFVFS
LDTSVSTAYLQISSLKAEDTSVYFCARLGYGKFYVMDYWGQGTSVTVSS (SEQ ID NO: 16)
QIQLVQSGPELKKPGASVKISCKASGYTFTNYGMNWVRQAPGQGLKWMGWINTYTGEPTYADDFTGRFVFS
LDTSVSTAYLQISSLKAEDTSVYFCARLGYGKFYVMDYWGQGTSVTVSS (SEQ ID NO: 17)
QIQLVQSGSELKKPGASVKISCKASGYTFTNYGMNWVRQAPGQGLKWMGWINTYTGEPTYADDFKGRFVFS
LDTSVSTAYLQISSLKAEDTSVYFCARLGYGKFYVMDYWGQGTSVTVSS (SEQ ID NO: 18)
QIQLVQSGPELKKPGASVKISCKASGYTFTNYGMNWVKQAPGQGLKWMGWINTYTGEPTYAQGFTGRFVFS
LDTSVSTAYLQISSLKAEDTSTYFCARLGYGKFYVMDYWGQGTSVTVSS (SEQ ID NO: 19)
QIQLVQSGPELKKPGASVKISCKASGYTFTSYAMNWVKQAPGQGLKWMGWINTYTGEPTYADDFKGRFVFS
LDTSVSTAYLQISSLKAEDTSVYFCARLGYGKFYVMDYWGQGTSVTVSS (SEQ ID NO: 20)
QIQLVQSGHEVKQPGASVKISCKASGYTFTNYGMNWVPQAPGQGLEWMGWINTYTGEPTYADDFKGRFVFS
LDTSASTAYLQISSLKAEDMSMYFCARLGYGKFYVMDYWGQGTSVTVSS (SEQ ID NO: 21)
QIQLVQSGHEVKQPGASVKISCKASGYTFTNYGMNWVPQAPGQGLEWMGWINTYTGEPTYAQGFTGRFVFS
LDTSASTAYLQISSLKAEDMSMYFCARLGYGKFYVMDYWGQGTSVTVSS (SEQ ID NO: 22)
QIQLVQSGHEVKQPGASVKISCKASGYTFTNYGMNWVKQAPGQGLKWMGWINTYTGEPTYADDFTGRFVFS
LDTSASTAYLQISSLKAEDMSMYFCARLGYGKFYVMDYWGQGTSVTVSS (SEQ ID NO: 23)
QIQLVQSGPEVKQPGASVKISCKASGYTFTNYGMNWVPQAPGQGLKWMGWINTYTGEPTYADDFTGRFVFS
LDTSASTAYLQISSLKAEDMSMYFCARLGYGKFYVMDYWGQGTSVTVSS (SEQ ID NO: 24)
QIQLVQSGPEVKQPGASVKISCKASGYTFTNYGMNWVKQAPGQGLKWMGWINTYTGEPTYAQGFTGRFVFS
LDTSASTAYLQISSLKAEDMSTYFCARLGYGKFYVMDYWGQGTSVTVSS (SEQ ID NO: 25)
QIQLVQSGPEVKQPGASVKISCKASGYSFTTYGMNWVKQAPGQGLEWMGWINTYTGEPTYADDFKGRFVFS
LDTSASTAYLQISSLKAEDMSTYFCARLGYGKFYVMDYWGQGTSVTVSS (SEQ ID NO: 26)
Light Chain CDRs
DVVMTQSPLSLPVTLGQPASISCRSSQSLVHSNGNTYLNWYQQRPGQSPRLLIYKVSNRFSGVPDRFSGSG
SGTDFTLKISRVEAEDVGVYFCSQSTHVTWTFGGGTKLEIK (SEQ ID NO: 27)
DVVMTQSPLSLPVTLGQPASISCRSSQSLVHSDGNTYLHWYQQRPGQSPRLLIYKVSNRFSGVPDRFSGSG
SGTDFTLKISRVEAEDVGVYFCSQSTHVTWTFGGGTKLEIK (SEQ ID NO: 28)
DVVMTQSPLSLPVTLGQPASISCRSSQSLVHSNGNTYLHWYQQRPGQSPRLLIYKVSNRDSGVPDRFSGSG
SGTDFTLKISRVEAEDVGVYFCSQSTHVTWTFGGGTKLEIK (SEQ ID NO: 29)
DVVMTQSPLSLPVTLGQPASISCRSSQSLVHSNGNTYLHWFQQRPGQSPRLLIYKVSNRFSGVPDRFSGSG
SGTDFTLKISRVEAEDVGVYFCSQSTHVTWTFGGGTKLEIK (SEQ ID NO: 30)
DIVMTQTPLSLSVTPGQPASISCKSSQSLVHSNGNTYLHWYLQKPGQSPQLLIYKVSNRFSGVPDRFSGSG
SGTDFTLKISRVEAEDVGVYYCSQSTHVTWTFGGGTKLEIK (SEQ ID NO: 31)
DVVMTQSPLSLPVTLGQPASISCRSSQSLVHSNGNTYLHWFQQRPGQSPRRLIYKVSNRFSGVPDRFSGSG
SGTDFTLKISRVEAEDVGVYYCSQSTHVTWTFGGGTKLEIK (SEQ ID NO: 32)
DVVMTQSPLSLPVTLGQPASISCRSSQSLVHSNGNTYLHWFLQRPGQSPRLLIYKVSNRFSGVPDRFSGSG
SGTDFTLKISRVEAEDVGVYYCSQSTHVTWTFGGGTKLEIK (SEQ ID NO: 33)
DVVMTQSPLSLPVTLGQPASISCRSSQSLVHSNGNTYLHWYQQRPGQSPRLLIYKVSNRFSGVPDRFSGSG
SGTDFTLKISRVEAEDVGVYYCSQSTHVTWTFGGGTKLEIK (SEQ ID NO: 34)

In some embodiments of any of the aspects, the antibody, antibody reagent, antigen-binding portion thereof, or CAR that specifically binds an CHI3L1 polypeptide binds specifically to an epitope selected from SEQ ID NOs: 13-24 disclosed in U.S. Pat. No. 10,253,111, reproduced in Table 4 as SEQ ID NOs: 35-46, respectively. In some embodiments of any of the aspects, the antibody, antibody reagent, antigen-binding portion thereof, or CAR that specifically binds a CHI3L1 polypeptide binds specifically to the epitope of SEQ ID NO: 35 in Table 4.

TABLE 4
CHI3L1 Epitopes

Phe Arg Gly Gln Glu Asp Ala Ser Pro Asp Arg Phe (SEQ ID NO: 35)
Arg Gly Ala Thr Val His Arg Ile Leu Gly Gln Gln (SEQ ID NO: 36)
Ala Ser Ser Glu Thr Gly Val Gly Ala Pro Ile Ser (SEQ ID NO: 37)
Ile Lys Glu Ala Gln Pro Gly Lys Lys Gln Leu Leu (SEQ ID NO: 38)
Ser Asn Asp His Ile Asp Thr Trp Glu Trp Asn Asp (SEQ ID NO: 39)
Tyr Pro Gly Arg Arg Asp Lys Gln His Phe Thr Thr (SEQ ID NO: 40)
Leu Arg Leu Gly Ala Pro Ala Ser Lys Leu Val Met (SEQ ID NO: 41)
Pro Gly Ile Pro Gly Arg Phe Thr Lys Glu Ala Gly (SEQ ID NO: 42)
Gly Ser Gln Arg Phe Ser Lys Ile Ala Ser Asn Thr (SEQ ID NO: 43)
Gly Lys Val Thr Ile Asp Ser Ser Tyr Asp Ile Ala (SEQ ID NO: 44)
Gly Met Leu Asn Thr Leu Lys Asn Arg Asn Pro Asn (SEQ ID NO: 45)
Ser Asn Thr Gln Ser Arg Arg Thr Phe Ile Lys Ser (SEQ ID NO: 46)

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen (e.g., CHI3L1). Such conservatively modified variants are in addition to and do not exclude polymorphic variants, interspecies homologs, and alleles consistent with the disclosure.

Examples of substitution variants include conservative substitution of amino acids, e.g., in a V_{H} or V_{L}, domain, that do not alter the sequence of a CDR. A conservative substitution in a sequence not comprised by a CDR can be a substitution relative to a wild-type or naturally-occurring sequence, e.g., human or murine framework and/or constant regions of an antibody sequence.

A given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., antigen-binding activity and specificity of a native or reference polypeptide is retained.

Amino acids can be grouped according to similarities in the properties of their side chains (BIOCHEMISTRY, 2^{nd} edition, (1975)⁵¹ at pp. 73-75): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

A variant amino acid or DNA sequence preferably is at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required.

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

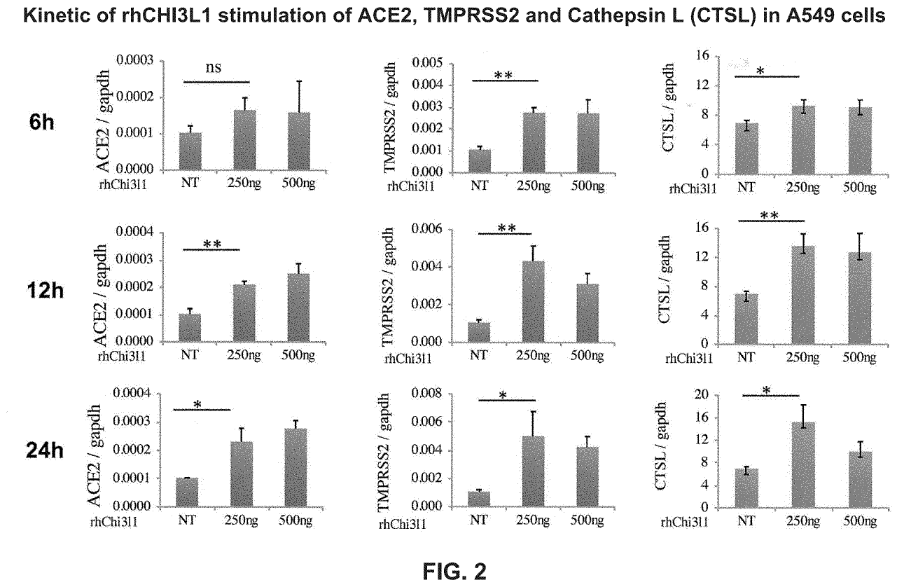

In particular embodiments wherein an antibody, antigen-binding portion thereof, or CAR as described herein comprises at least one CDR which is not identical to the sequence of CHI3L1 CDR provided herein, the amino acid sequence of that at least one CDR can be selected by methods well known to one of skill in the art. For example, Fujii (2004),⁵² particularly at FIG. 2 and Section 3.3, which describes methods of generating a library for any CDR of interest. This allows one of ordinary skill in the art to identify alternative CDRs, including conservative substitution variants of the specific CDR sequences described herein, which, when present in an antibody or antigen-binding portion thereof as described herein, will result in an antigen or antigen-binding portion thereof which will bind a cell surface antigen. The method described in Fujii also permits one of ordinary skill in the art to screen for a light chain sequence which will give the desired binding behavior when combined with a known heavy chain fragment and vice versa.

In some embodiments, a CAR comprises an extracellular domain comprising an anti-CHI3L1 antibody or antigen-binding portion thereof that binds one or more epitopes of a CHI3L1 polypeptide; a transmembrane domain, one or more intracellular co-stimulatory signaling domains, and a primary signaling domain. Exemplary anti-CHI3L1 antibodies and antigen-binding portions thereof, as well as exemplary epitopes, are described elsewhere herein.

As used herein, “chimeric antigen receptor” or “CAR” refers to an artificially constructed hybrid polypeptide comprising an antigen-binding domain (e.g., an antigen-binding portion of an antibody (e.g., a scFv)), a transmembrane domain, and a T cell signaling and/or T cell activation domain. CARs have the ability to redirect T cell specificity and reactivity toward a selected target in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries), in various embodiments, this scFv is fused to a transmembrane domain and then to an intracellular signaling domain. “First-generation” CARs include those that solely provide CD3zeta (CD3ζ) signals upon antigen binding, “Second-generation” CARs include those that provide both co-stimulation (e.g., CD28 or CD 137) and activation (CD3ζ). “Third-generation” CARs include those that provide multiple costimulatory (e.g., CD28 and CD 137) domains and activation domains (e.g., CD3ζ). In various embodiments, the CAR is selected to have high affinity or avidity for the antigen. Further discussion of CARs can be found, e.g., in Maus et al. (2014);⁵³ Reardon et al. (2014);⁵⁴ Hoyos et al. (2012);⁵⁵ Byrd et al. (2014);⁵⁶ Maher and Wilkie (2009);⁵⁷ and Tamada et al. (2012).⁵⁸

In some embodiments of any of the aspects, a CAR comprises an extracellular binding domain that comprises a humanized CHI3L1-specific binding domain; a transmembrane domain; one or more intracellular co-stimulatory signaling domains; and a primary signaling domain. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., CHI3L1. The binding domain may be derived either from a natural, synthetic, semi-synthetic, or recombinant source.

In some embodiments, the CARs contemplated herein may comprise linker residues between the various domains, e.g., added for appropriate spacing and conformation of the molecule. In particular embodiments the linker is a variable region linking sequence. A “variable region linking sequence,” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, can comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In particular embodiments, the binding domain of the CAR is followed by one or more “spacer domains,” which refers to the region that moves the antigen binding domain away from the effector cell surface to enable proper cell/cell contact, antigen binding and activation. The hinge domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8a, CD4, CD28 and CD7, which may be wild-type hinge regions from these molecules or may be altered. In another embodiment, the hinge domain comprises a CD8a hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T cell receptor, CD3ε, CD3ζ, CD4, CD5, CD8a, CD9, CD 16, CD22, CD27, CD28, CD33, CD37, CD45, CD64, CD80, CD86, CD134, CD137, CD152, CD154, and PD1.

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Illustrative examples of ITAM containing primary signaling domains that are of particular use in the invention include those derived from TCRζ, FcRγ, FcRβ, CD3γ, CD3δ, CD3ε, CD3ζ, CD22, CD79a, CD79b, and CD66d.

As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In one embodiment, a CAR comprises one or more co-stimulatory signaling domains selected from the group consisting of CD28, CD137, and CD134, and a CD3ζ primary signaling domain.

In some embodiments, an antibody-drug conjugate is provided. In particular embodiments, an antibody-drug conjugate comprises an antibody, antibody reagent, or antigen-binding portion thereof as described herein. In some embodiments, the antibody-drug conjugate comprises a therapeutic agent directly conjugated and/or bound to an antibody or antigen-binding portion thereof. In some embodiments, binding can be non-covalent, e.g., by hydrogen bonding, electrostatic, or van der Waals interactions; however, binding may also be covalent. By “conjugated” is meant the covalent linkage of at least two molecules. In some embodiments, the composition can be an antibody-drug conjugate.

In some embodiments, an antibody, antibody reagent, or antigen-binding portion thereof can be bound to and/or conjugated to multiple therapeutic molecules. In some embodiments, an antibody-drug conjugate can be bound to and/or conjugated to multiple therapeutic molecules. In some embodiments, the ratio of a given therapeutic molecule to an antibody or antigen-binding portion thereof can be from about 1:1 to about 1,000:1, e.g., a single antibody reagent molecule can be linked to, conjugated to, etc. from about 1 to about 1,000 individual therapeutic molecules.

In some embodiments, an antibody, or antigen-binding portion thereof, and the therapeutic agent can be present in a scaffold material. Scaffold materials suitable for use in therapeutic compositions are known in the art and can include, but are not limited to, a nanoparticle; a matrix; a hydrogel; and a biomaterial, biocompatible, and/or biodegradable scaffold material. As used herein, the term “nanoparticle” refers to particles that are on the order of about 10⁻⁹ or one to several billionths of a meter. The term “nanoparticle” includes nanospheres; nanorods; nanoshells; and nanoprisms; these nanoparticles may be part of a nanonetwork.

The term “nanoparticles” also encompasses liposomes and lipid particles having the size of a nanoparticle. As used herein, the term “matrix” refers to a 3-dimensional structure comprising the components of a composition described herein (e.g., an antibody or antigen-binding portion thereof). Non-limiting examples of matrix structures include foams; hydrogels; electrospun fibers; gels; fiber mats; sponges; 3-dimensional scaffolds; non-woven mats; woven materials; knit materials; fiber bundles; and fibers and other material formats. See, e.g., Rockwood et al. (2011)⁵⁹ and U.S. Patent Publications 2011/0167602;⁶⁰ 2011/0009960;⁶¹ 2012/0296352;⁶² and U.S. Pat. No. 8,172,901.⁶³ The structure of the matrix can be selected by one of skill in the art depending upon the intended application of the composition, e.g., electrospun matrices can have greater surface area than foams.

In some embodiments, the scaffold is a hydrogel. As used herein, the term “hydrogel” refers to a three-dimensional polymeric structure that is insoluble in water but which is capable of absorbing and retaining large quantities of water to form a stable, often soft and pliable, structure. In some embodiments, water can penetrate in between the polymer chains of the polymer network, subsequently causing swelling and the formation of a hydrogel. In general, hydrogels are superabsorbent. Hydrogels have many desirable properties for biomedical applications. For example, they can be made nontoxic and compatible with tissue, and they are highly permeable to water, ions, and small molecules. Hydrogels are super-absorbent (they can contain over 99% water) and can be comprised of natural (e.g., silk) or synthetic polymers, e.g., PEG.

As used herein, “biomaterial” refers to a material that is biocompatible and biodegradable. As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo. As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding amino acid sequence variants of antibodies are prepared by a variety of methods known in the art. These methods include, but are not limited to preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations can be used to construct nucleic acid sequences which encode a monoclonal antibody molecule, antibody reagent, antigen binding region thereof, or CAR.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art.

In some embodiments, a nucleic acid encoding an antibody, antibody reagent, antigen-binding portion thereof, or CAR as described herein is comprised by a vector. In some of the aspects described herein, a nucleic acid sequence encoding an antibody, antibody reagent, antigen-binding portion thereof, or CAR as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody, antigen-binding portion thereof, or CAR as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In one aspect of any of the embodiments, described herein is a cell comprising an antibody, antibody reagent, antigen-binding portion thereof, or CAR as described herein, or a nucleic acid encoding such an antibody, antibody reagent, antigen-binding portion thereof, or CAR.

The expression of an antibody, antibody reagent, antigen-binding portion thereof, or CAR as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence. Moreover, problems associated with retention of initiation codon-derived methionine residues in direct yeast (or bacterial) expression maybe avoided. Any of a series of yeast gene expression systems incorporating promoter and termination elements from the actively expressed genes coding for glycolytic enzymes produced in large quantities when yeast are grown in mediums rich in glucose can be utilized to obtain recombinant antibodies or antigen-binding portions thereof as described herein. Known glycolytic genes can also provide very efficient transcriptional control signals. For example, the promoter and terminator signals of the phosphoglycerate kinase gene can be utilized.

Production of antibodies or antigen-binding portions thereof as described herein in insects can be achieved. For example, by infecting the insect host with a baculovirus engineered to express a transmembrane polypeptide by methods known to those of ordinary skill in the art.

In some embodiments, the introduced nucleotide sequence is incorporated into a plasmid or viral vector capable of autonomous replication in the recipient host. Any of a wide variety of vectors can be employed for this purpose and are known and available to those or ordinary skill in the art. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli., for example. Other gene expression elements useful for the expression of cDNA encoding antibodies, antigen-binding portions thereof, or CARs include, but are not limited to (a) viral transcription promoters and their enhancer elements, such as the SV40 early promoter, Rous sarcoma virus LTR, and Moloney murine leukemia virus; (b) splice regions and polyadenylation sites such as those derived from the SV40 late region, and (c) polyadenylation sites such as in SV40. Immunoglobulin cDNA genes can be expressed, e.g., using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA, the transcriptional promoter can be human cytomegalovirus, the promoter enhancers can be cytomegalovirus and mouse/human immunoglobulin, and mRNA splicing, and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

A gene is assembled in, or inserted into, an expression vector. Recipient cells capable of expressing the chimeric immunoglobulin chain gene product are then transfected singly with an antibody, antigen-binding portion thereof, or CAR, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the genes encoding the antibody, antigen-binding portion thereof, CAR, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the genes in a bacterial system. The genes so produced and amplified in a bacterial host are subsequently used to co-transfect a eukaryotic cell, and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the genes can be assembled on the same expression vector.

For transfection of the expression vectors and production of the antibodies, antibody reagents, antigen-binding portions thereof, or CARs described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, antigen-binding portion thereof, and/or CAR as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment, as known to one of ordinary skill in the art.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives for hybridoma-based production systems. The main advantages of transgenic animals are potential high yields from renewable sources.

In one aspect, a cell comprising an isolated antibody, antigen-binding portion thereof, or CAR as described herein is provided. In some embodiments, the isolated antibody, antigen-binding portion thereof, or CAR as described herein is expressed on the cell surface. In some embodiments, the cell comprises a nucleic acid encoding an isolated antibody, antigen-binding portion thereof, or CAR as described herein.

In some embodiments, the cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the cell is a T cell; a NK cell; a NKT cell; lymphocytes, such as B cells and T cells; and myeloid cells, such as monocytes, macrophages, eosinophils, mast cells, basophils, and granulocytes.

In particular embodiments, a cell (e.g., an immune cell) is transduced with a retroviral vector, e.g., a lentiviral vector, encoding a CAR. For example, an immune effector cell is transduced with a vector encoding a CAR that comprises an anti-CHI3L1 antibody or antigen binding portion thereof that binds a CHI3L1 polypeptide, with an intracellular signaling domain of CD3ζ, CD28, 4-1BB, Ox40, or any combinations thereof. Thus, these transduced cells can elicit a CAR-mediated cytotoxic response.

Retroviruses are a common tool for gene delivery. In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising a CAR to a cell.

Retroviral vectors and more particularly lentiviral vectors may be used in practicing particular embodiments of the present invention. Accordingly, the term “retrovirus” or “retroviral vector”, as used herein is meant to include “lentivirus” and “lentiviral vectors” respectively.

### CHI3L1 Phosphorylation Inhibitors/CDK Inhibitors

In one aspect of any of the embodiments described herein, the methods of the present invention can further include the administration of an inhibitor of CHI3L1 phosphorylation. In some embodiments, the CHI3L1 phosphorylation inhibitor is an CDK (cyclin-dependent kinase) inhibitor. A CDK inhibitor is any chemical that inhibits the function of CDKs. They are been used to treat cancers by preventing over proliferation of cancer cells. In our laboratory, the phosphorylation of CHI3L1 was shown to be blocked by several CDK inhibitors, including flavopiridol, a broad spectrum CDK inhibitor. This CDK inhibitor was also shown to decrease the induction of ACE2 and SAPs.

CDK inhibitors have been categorized into three groups based on their target specificity: (i) broad CDK inhibitors, which include compounds targeting a broad spectrum of CDKs; (ii) specific CDK inhibitors, which include compounds targeting a specific CDK isoforms; and (iii) multiple target inhibitors, which include compounds targeting CDKs as well as additional kinases such as VEGFR or PDGFR.⁶⁴ CDK inhibitors that can be used in the methods of the present invention include, but are not limited to, CDK inhibitors listed in Table 1.

TABLE 1
Inhibitory potency of several CDK inhibitors⁶⁵

| CDK Inhibitor | CDK1 | CDK2 | CDK3 | CDK4 | CDK5 | CDK6 | CDK7 | CDK8 | CDK9 | CLK |
|---|---|---|---|---|---|---|---|---|---|---|
| AT7519 | ++ | ++ | | ++ | +++ | ++ | + | | ++++ | |
| BS-181 HCl | | | | | | | +++ | | | |
| Flavopiridol | +++ | +++ | | +++ | | ++ | + | | | |
| Flavopiridol HCl | +++ | +++ | + | +++ | | +++ | + | | | |
| JNJ-7706621 | ++++ | ++++ | | + | | ++ | | | | |
| Palbociclib HCl | | | | +++ | | +++ | | | | |
| PHA-793887 | ++ | ++++ | | ++ | ++++ | | ++++ | | ++ | |
| Roscovitine | | + | | | ++ | | | | | |
| SNS-032 | + | +++ | | + | + | | ++ | | ++++ | |
| A-674563 | | ++ | | | | | | | | |
| Milciclib | + | +++ | | ++ | + | | ++ | | | |
| AZD5438 | +++ | ++++ | ++ | | | | | | +++ | |
| Dinaciclib | ++++ | ++++ | | | ++++ | | | | ++++ | |
| BMS-265246 | ++++ | ++++ | | ++ | | | | | | |
| PHA-767491 | ++ | ++ | | | + | | | | +++ | |
| MK-8776 | | ++ | | | | | | | | |
| R547 | ++++ | ++++ | | ++++ | | | | | | |
| Kenpaulione | + | + | | | + | | | | | |
| AT7519 HCl | ++ | ++ | + | ++ | +++ | ++ | | | ++++ | |
| CGP60474 | +++ | +++ | | | | | | | | |
| Wogonin | | | | | | | | | ◯ | |
| Purvalanol B | | +++ | | | ++++ | | | | | |
| NU 6102 | +++ | ++++ | | + | | | | | | |
| LY2835219 (Abemaciclib) | | | | ++++ | | +++ | | | | |
| P276-00 | ++ | ++ | | ++ | | + | + | | +++ | |
| Ribociclib | | | | ◯ | | | | | | |
| TG003 | | | | | | | | | | +++ |
| Palbociclib Isethionate | | | | ++++ | | +++ | | | | |
| AMG-925 | + | | | ++++ | | | | | | |
| NU6027 | + | | | | | | | | | |
| THZI | | | | | | | ++++ | | | |
| LDC000067 | + | | | + | | | | | +++ | |
| ML167 | | | | | | | | | | ++ |
| SU9516 | +++ | +++ | | ++ | | | | | | |
| Ro-3306 | +++ | | | | | | | | | |
| CVT 313 | + | + | | + | | | | | | |
| NVP-LCQ195 | ++++ | +++ | ◯ | | ++++ | ◯ | ◯ | | ◯ | ◯ |
| Purvalanol A | | +++ | | + | | | | | | |
| NU2058 | + | + | | | | | | | | |
| LY2857785 | | | | | | | + | +++ | +++ | |
| K03861 | | ++++ | | | | | | | | |
| Indirubin | ++++ | | | | | | | | | |

+ is used to indicate the potency of the inhibitor; ++++ > +++ > ++ > +
◯ refers to compounds with inhibitory effects on the related isoform, but without specific value.

In some embodiments, the CDK inhibitor administered is a broad CDK inhibitor. In some embodiments, the CDK inhibitor administered is a specific CDK inhibitor. In some embodiments, the CDK inhibitor administered is a multiple target inhibitor.

In some embodiments, the CDK inhibitor administered is a CDK inhibitor with potency for the CDK1, CDK2, CDK3, CDK4, CDK5, CDK6, CDK7, CDK8, CDK9, and/or CLK isomers. In some embodiments, the CDK inhibitor administered with KSM is a CDK inhibitor with potency for the CDK1 isomer and/or the CDK5 isomer.

In some embodiments, the CDK inhibitor administered with KSM is a CDK inhibitor selected from the group consisting of: Flavopiridol, Flavopiridol HCl, AT7519, BS-181 HCl, JNJ-7706621, Palbociclib HCl, PHA-793887, Roscovitine, SNS-032, A-674563, Milciclib, AZD5438, Dinaciclib, BMS-265246, PHA-767491, MK-8776, R547, Kenpaulione, AT7519 HCl, CGP60474, Wogonin, Purvalanol B, NU 6102, LY2835219 (Abemaciclib), P276-00, Ribociclib, TG003, Palbociclib Isethionate, AMG-925, NU6027, THZI, LDC000067, ML167, SU9516, Ro-3306, CVT 313, NVP-LCQ195, Purvalanol A, NU2058, LY2857785, K03861, and/or Indirubin.

### Flavopiridol

Flavopiridol Hydrochloride (aka Alvocidib Hydrochloride; L86-8275 Hydrochloride; HMR-1275 Hydrochloride, shown as Formula I) is a broad inhibitor of CDK, competing with ATP to inhibit CDK isomers including CDK1, CDK2, CDK4 with an IC₅₀ of 30, 170, and 100 nM, respectively. The compound is a synthetic analog of natural product rohitukine which was initially extracted from Amoora rohituka [syn. Aphanamixis polystachya] and later from Dysoxylum binectariferum.^{66,67}

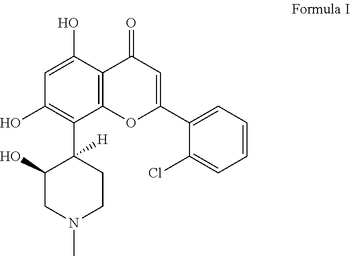 [SMILES: CN1CC[C@H](c2c(O)cc(O)c3c(=O)cc(-c4ccccc4Cl)oc23)[C@H](O)C1]

In some embodiments, the CDK inhibitor administered with KSM is a Flavopiridol or Flavopiridol HCl.

### Kasugamycin (KSM)

In another aspect of any of the embodiments described herein, the methods of the present invention can further include the administration of one or more inhibitor of CHI3L1 and chitinase 1 such as kasugamycin.

Kasugamycin (e.g., a compound of Formula II) is an amino cyclitol glycoside that is isolated from Streptomyces kasugaensis and exhibits antibiotic and fungicidal properties. Like many of the known natural antibiotics, kasugamycin inhibits proliferation of bacteria by tampering with their ability to make new proteins, the ribosome being the major target. Kasugamycin can also be referred to in the art as 2-amino-2-[(2R,3S,5S,6R)-5-amino-2-methyl-6-[(2R,3S,5S,6S)-2,3,4,5,6-pentahydroxycyclohexyl]oxyoxan-3-yl]iminoacetic acid; Kasumin; or 3-O-[2-Amino-4-[(carboxyiminomethyl)amino]-2,3,4,6-tetradeoxy-D-arabino-hexopyranosyl]-D-chiro-inositol.

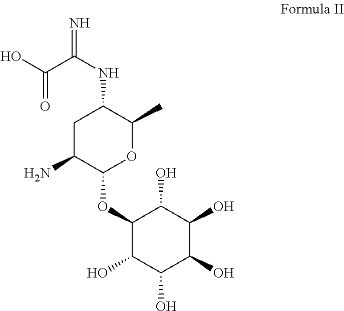 [SMILES: C[C@H]1O[C@H](O[C@H]2[C@H](O)[C@@H](O)[C@@H](O)[C@H](O)[C@@H]2O)[C@@H](N)C[C@@H]1NC(=N)C(=O)O]

Non-limiting examples of kasugamycin derivatives include those described in U.S. Pat. Nos. 3,968,100; 4,554,269; 5,317,095; and 3,480,614.⁶⁸ As used herein, a molecule is said to be a “derivative” of another molecule when it contains additional chemical moieties not normally a part of the molecule and/or when it has been chemically modified. Such moieties can improve the molecule's expression levels, enzymatic activity, solubility, absorption, biological half-life, etc. The moieties can alternatively decrease the toxicity of the molecule, eliminate or attenuate any undesirable side effect of the molecule, etc. Moieties capable of mediating such effects are disclosed in REMINGTON'S PHARMACEUTICAL SCIENCES (1990).⁶⁹ A “variant” of a molecule is meant to refer to a molecule substantially similar in structure and function to either the entire molecule, or to a fragment thereof. A molecule is said to be “substantially similar” to another molecule if both molecules have substantially similar structures and/or if both molecules possess a similar biological activity. Thus, provided that two molecules possess a similar activity, they are considered variants as that term is used herein even if the structure of one of the molecules not found in the other, or if the structure is not identical. An “analog” of a molecule is meant to refer to a molecule substantially similar in function to either the entire molecule or to a fragment thereof.

We have previously demonstrated in our laboratory that kasugamycin displays unexpected activity in inhibiting the mechanisms of fibrosis, an activity unique to kasugamycin and not displayed by other aminoglycoside antibiotics.⁷⁰ We have shown that kasugamycin has the ability to inhibit CHI3L1 and its cousin molecule chitinase 1. It has been reported to have antiviral properties. Based on these observations, it was hypothesized that a therapeutic agent inhibiting CHI3L1 and chitinase 1 might be useful in the prevention or treatment of the deleterious effects of Covid-19 infections.

In some embodiments of any of the aspects, an effective dose of a composition comprising kasugamycin or a derivative, analog, or variant thereof as described herein can be administered to a patient once. In some embodiments of any of the aspects, an effective dose of a composition comprising kasugamycin or a derivative, analog, or variant thereof can be administered to a patient repeatedly. For systemic administration, subjects can be administered a therapeutic amount of a composition comprising kasugamycin or a derivative, analog, or variant thereof, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, the kasugamycin or a derivative, analog, or variant thereof as described herein can be administered at a dose of more than about 50 mg/kg. In some embodiments of any of the aspects, the kasugamycin or a derivative, analog, or variant thereof as described herein can be administered at a dose of about 100 mg/kg or greater. In some embodiments of any of the aspects, the kasugamycin or a derivative, analog, or variant thereof as described herein can be administered at a dose from about 50 mg/kg to about 500 mg/kg. In some embodiments of any of the aspects, the kasugamycin or a derivative, analog, or variant thereof as described herein can be administered at a dose from about 50 mg/kg to about 1,000 mg/kg. In some embodiments of any of the aspects, the kasugamycin or a derivative, analog, or variant thereof as described herein can be administered at a dose from about 100 mg/kg to about 500 mg/kg.

In some embodiments of any of the aspects, after an initial treatment regimen, the treatments can be administered on a less frequent basis. For example, after daily treatments for two weeks, treatment biweekly for three months, treatment can be repeated once per month, for six months or a year or longer. Treatment according to the methods described herein can reduce levels of a marker or symptom of a condition, e.g., kasugamycin or a derivative, analog, or variant thereof by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to kasugamycin or a derivative, analog, or variant thereof. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising kasugamycin or a derivative, analog, or variant thereof can be administered over a period of time, such as over a 5 minute, 10 minute, 15 minute, 20 minute, or 25 minute period.

The dosage ranges for the administration of kasugamycin or a derivative, analog, or variant thereof, according to the methods described herein depend upon, e.g., the form of the active ingredient, its potency, and the extent to which symptoms, markers, or indicators of a condition described herein are desired to be reduced, for example the percentage reduction desired for fibrosis. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of kasugamycin or a derivative, analog, or variant thereof in, e.g., the treatment of a condition described herein, or to induce a response as described herein (e.g., a decrease of chitinase activity) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., chitinase activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., collagen levels, degree of fibrosis, and/or BAL cell recovery). It is well within the ability of one skilled in the art to monitor efficacy of administration and/or treatment by measuring any one of such parameters, or any combination of parameters. Efficacy can be assessed in animal models of a condition described herein, for example treatment of mouse models of pulmonary fibrosis. When using an experimental animal model, efficacy of treatment is evidenced when a statistically significant change in a marker is observed, e.g., collagen levels, and/or chitinase activity.

### Pharmaceutical Compositions

In one aspect of any of the embodiments, described herein is a composition comprising an antibody, antibody reagent, antigen-binding portion thereof, or CAR as described herein or a nucleic acid encoding an antibody, antibody reagent, antigen-binding portion thereof, or CAR as described herein, or a cell as described herein. In some embodiments, the composition is a pharmaceutical composition. As used herein, the term “pharmaceutical composition” refers to the active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic agent or composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the invention that will be effective in the treatment of a particular disorder or condition will depend on the nature of the disorder or condition and can be determined by standard clinical techniques.

In some embodiments, the composition comprising an antibody, antibody reagent, antigen-binding portion thereof, or CAR as described herein or a nucleic acid encoding an antibody, antibody reagent, antigen-binding portion thereof, or CAR as described herein can be a lyophilisate.

In some embodiments, the technology described herein relates to a syringe or catheter, including an organ-specific catheter (e.g., renal catheter, biliary catheter, cardiac catheter, etc.), comprising a therapeutically effective amount of a composition described herein.

In one aspect, described herein is a method of inhibiting or killing a CHI3L1+ cell, the method comprising contacting the cell with an isolated antibody, antibody reagent, antigen-binding portion thereof, or CAR as described herein, a nucleic acid encoding such polypeptides, a cell comprising such a polypeptide or nucleic acid, or a composition comprising such a polypeptide or nucleic acid. Inhibiting a CHI3L1+ cell can comprise inhibiting the metabolic activity, metastasis, and/or proliferation of the cell. Assays for measuring metabolic activity, metastasis (e.g., migration assays) and proliferation are well known in the art. Similarly, assays for measuring killing of CHI3L1+ cells, e.g., cell viability assays are well known in the art.

As used herein, a “CHI3L1+” cell is a cell expressing an increased level of CHI3L1+, e.g., as compared to a healthy cell of the same type or an average level of CHI3L1+ found in healthy cells of the same type.

In some embodiments of any of the aspects described herein, a subject administered a composition described herein can be a subject determined to have an elevated level of CHI3L1 or a level of CHI3L1 that is increased compared to a prior assessment of the level in that subject. In some embodiments of any of the aspects, the elevated level of CHI3L1 is the level of circulating CHI3L1.

In some embodiments of any of the aspects described herein, the method comprising administering a composition as described herein can further comprise a first step of identifying a subject having an elevated level of CHI3L1. In some embodiments of any of the aspects, the elevated level of CHI3L1 is the level of circulating CHI3L1.

As used herein, a “CHI3L1+” cell is a cell expressing an increased level of CHI3L1+, e.g., as compared to a healthy cell of the same type or an average level of CHI3L1 found in healthy cells of the same type. In some embodiments of any of the aspects, an increased level of CHI3L1 can be a level which is at least 1.5× the level found in a reference, e.g., 1.5×, 2×, 3×, 4×, 5× or greater than the reference level.

In one aspect, the technology described herein relates to a method comprising administering an antibody, antibody reagent, antigen-binding portion thereof, or CAR as described herein or a nucleic acid encoding an antibody, antibody reagent, antigen-binding portion thereof, or CAR as described herein to a subject. In some embodiments, the subject is in need of treatment for a Covid-19 infection. In some embodiments, the subject is in need of treatment for severe symptoms of Covid-19 including, but not limited to, acute respiratory syndrome (SARS), acute respiratory distress syndrome (ARDS), acute liver injury, acute cardiac injury, acute kidney injury, septic shock, disseminated intravascular coagulation, blood clots, multisystem inflammatory syndrome, and rhabdomyolysis. In some embodiments, the method is a method of treating a subject. In some embodiments, the method is a method of treating a Covid-19 infection in a subject.

In one aspect, the technology described herein relates to a method comprising administering an antibody, antibody reagent, antigen-binding portion thereof, or CAR as described herein or a nucleic acid encoding an antibody, antibody reagent, antigen-binding portion thereof, or CAR as described herein to a subject.

In one aspect, described herein is a method of treating a Covid-19 infection in a subject in need thereof, the method comprising administering a cell as described herein, e.g., a cell comprising an antibody, antibody reagent, antigen-binding portion thereof, or CAR as described herein. In some embodiments, the cell is an immune cell.

In one aspect, described herein is a method of treating a Covid-19 infection in a subject in need thereof, the method comprising administering a nucleic acid as described herein or an immune cell comprising the nucleic acid to the subject, wherein the subject's immune cells are caused to express the polypeptide encoded by the nucleic acid. In some embodiments, the immune cell is a T cell. Nucleic acids can be targeted to particular cell types by, e.g., use of a cell-type specific promoter and/or a composition that selectively binds to the desired cell type. For example, conjugation of a nucleic acid to an aptamer can permit targeted delivery. See, e.g., McNamara, et al. (2006).⁷¹ In an alternative embodiment, the nucleic acid can be delivered using drug delivery systems such as a nanoparticle, a dendrimer, a polymer, liposomes, or a cationic delivery system. Positively charged cationic delivery systems facilitate binding of a nucleic acid molecule (negatively charged) and also enhance interactions at the negatively charged cell membrane to permit efficient uptake of a nucleic acid by the cell. Cationic lipids, dendrimers, or polymers, can either be bound to a nucleic acid, or induced to form a vesicle or micelle (see, e.g., Kim, et al. (2008)⁷²) that encases a nucleic acid. The formation of vesicles or micelles further prevents degradation of the nucleic acid when administered systemically. Methods for making and administering cationic-inhibitory nucleic acid complexes are well within the abilities of one skilled in the art. Some non-limiting examples of drug delivery systems useful for systemic delivery of nucleic acids include DOTAP Oligofectamine, “solid nucleic acid lipid particles”, cardiolipin, polyethyleneimine, Arg-Gly-Asp (RGD) peptides, and polyamidoamines. In some embodiments, a nucleic acid forms a complex with cyclodextrin for systemic administration. Methods for administration and pharmaceutical compositions of nucleic acids and cyclodextrins can be found in U.S. Pat. No. 7,427,605.⁷³ Targeted delivery of nucleic acids is described, for example in Ikeda and Taira (2006);⁷⁴ Soutschek et al. (2004);⁷⁵ and Lorenze et al. (2004).⁷⁶ By way of example, the nucleic acid can be targeted to immune cells by encapsulating the inhibitor in a liposome comprising ligands of receptors expressed on immune cells, e.g., TCRs. In some embodiments, the liposome can comprise aptamers specific for immune cells.

In some embodiments, the methods described herein relate to CAR-T cell therapy. CAR-T cell and related therapies relate to adoptive cell transfer of immune cells (e.g., T cells) expressing a CAR that binds specifically to a targeted cell type (e.g., cells expressing ACE2) to treat a subject. In some embodiments, the cells administered as part of the therapy can be autologous to the subject. In some embodiments, the cells administered as part of the therapy are not autologous to the subject. In some embodiments, the cells are engineered and/or genetically modified to express the CAR.

It can generally be stated that a pharmaceutical composition comprising the cells, e.g., T cells or immune cells, described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/mL and generally is greater than 10⁷ cells/mL, generally 10⁸ cells/mL or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response. In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. In some embodiments, one dose of cells can be administered. In some embodiments, the dose of cells can be repeated, e.g., once, twice, or more. In some embodiments, the dose of cells can be administered on, e.g., a daily, weekly, or monthly basis.

The dosage ranges for the agent depend upon the potency and encompass amounts large enough to produce the desired effect e.g., a reduction or elimination of one or more of the Covid-19 symptoms. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In some embodiments, the doses are given once a day, or multiple times a day, for example but not limited to three times a day. In some embodiments, the doses recited above are administered daily for several weeks or months. The duration of treatment depends upon the subject's clinical progress and responsiveness to therapy.

In some embodiments, the dose can be from about 2 mg/kg to about 15 mg/kg. In some embodiments, the dose can be about 2 mg/kg. In some embodiments, the dose can be about 4 mg/kg. In some embodiments, the dose can be about 5 mg/kg. In some embodiments, the dose can be about 6 mg/kg. In some embodiments, the dose can be about 8 mg/kg. In some embodiments, the dose can be about 10 mg/kg. In some embodiments, the dose can be about 15 mg/kg. In some embodiments, the dose can be from about 100 mg/m² to about 700 mg/m². In some embodiments, the dose can be about 250 mg/m². In some embodiments, the dose can be about 375 mg/m². In some embodiments, the dose can be about 400 mg/m². In some embodiments, the dose can be about 500 mg/m2.

In some embodiments, the dose can be administered intravenously. In some embodiments, the intravenous administration can be an infusion occurring over a period of from about 10 minute to about 3 hours. In some embodiments, the intravenous administration can be an infusion occurring over a period of from about 30 minutes to about 90 minutes.

In some embodiments, the dose can be administered about daily. In some embodiments, the dose can be administered weekly. In some embodiments, the dose can be administered weekly for from about 1 week to about 12 weeks. In some embodiments, the dose can be administered about every two days. In some embodiments, the dose can be administered about every three days. In some embodiments, the dose can be from about 2 mg/kg to about 15 mg/kg administered about every two days. In some embodiments, the dose can be from about 2 mg/kg to about 15 mg/kg administered about every three days. In some embodiments, the dose can be from about 2 mg/kg to about 15 mg/kg administered intravenously about every two days. In some embodiments, the dose can be from about 2 mg/kg to about 15 mg/kg administered intravenously about every three days. In some embodiments, the dose can be from about 200 mg/m² to about 400 mg/m² administered intravenously about every day. In some embodiments, the dose can be from about 200 mg/m2 to about 400 mg/m² administered intravenously about every two days. In some embodiments, the dose can be from about 200 mg/m² to about 400 mg/m² administered intravenously about every three days. In some embodiments, a total of from about 2 to about 10 doses are administered. In some embodiments, a total of four doses are administered. In some embodiments, a total of five doses are administered. In some embodiments, a total of six doses are administered. In some embodiments, a total of seven doses are administered. In some embodiments, a total of eight doses are administered. In some embodiments, the administration occurs for a total of from about four weeks to about 12 weeks. In some embodiments, the administration occurs for a total of about six weeks. In some embodiments, the administration occurs for a total of about eight weeks. In some embodiments, the administration occurs for a total of about 12 weeks. In some embodiments, the initial dose can be from about 1.5 to about 2.5 fold greater than subsequent doses.

In some embodiments, the dose can be from about 1 mg to about 2000 mg. In some embodiments, the dose can be about 3 mg. In some embodiments, the dose can be about 10 mg. In some embodiments, the dose can be about 30 mg. In some embodiments, the dose can be about 1000 mg. In some embodiments, the dose can be about 2000 mg. In some embodiments, the dose can be about 3 mg given by intravenous infusion daily. In some embodiments, the dose can be about 10 mg given by intravenous infusion daily. In some embodiments, the dose can be about 30 mg given by intravenous infusion three times per week.

A therapeutically-effective amount is an amount of an agent that is sufficient to produce a statistically significant, measurable reduction of Covid-19 symptoms (efficacy measurements are described herein). Such effective amounts can be gauged in clinical trials as well as animal studies.

An agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. It is preferred that the compounds used herein are administered orally, intravenously or intramuscularly to a patient having a Covid-19 infection. Local administration directly to the subject's lungs is also specifically contemplated.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically-effective amount. The quantity to be administered and timing depends on the subject to be treated, capacity of the subject's system to utilize the active ingredient, and degree of therapeutic-effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In some embodiments, the methods further comprise administering the pharmaceutical composition described herein along with one or more additional therapeutic agents, biologics, drugs, or treatments as part of a combinatorial therapy. In some such embodiments, the therapeutic agent biologic, drug, or treatment is selected from the group consisting of: (i) an inhibitor CHI3L1 and chitinase 1; (ii) a CDK inhibitor; (iii) remdesivir; (ii) dexamethasone; (iii) REGEN-COV-2 (Regeneron); (iv) Baricitinib (Lilly); (v) Sotrovimab (Vir); (vi) PAXLOVID™ (Pfizer); and/or molnupiravir (Merck). In some embodiments, the inhibitor CHI3L1 and chitinase 1 is Kasugamycin. In some embodiments, the CDK inhibitor is Flavopiridol.

The efficacy of a given treatment for, e.g., Covid-19, can be determined by the skilled clinician. However, a treatment is considered “effective treatment,” as the term is used herein, if any one or all of the signs or symptoms of e.g., breathing is altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

An effective amount for the treatment of a disease means that amount which, when administered to a mammal in need thereof, is sufficient to result in effective treatment as that term is defined herein, for that disease. Efficacy of a therapeutic agent can be determined by assessing physical indicators of, for example a Covid-19 infection, e.g., improved breathing, prevention or decrease in lung inflammation, tissue injury, and pulmonary fibrosis, acute respiratory syndrome (SARS), acute respiratory distress syndrome (ARDS), acute liver injury, acute cardiac injury, acute kidney injury, septic shock, disseminated intravascular coagulation, blood clots, multisystem inflammatory syndrome, and rhabdomyolysis, etc.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention, and are not intended to limit the invention.

### Example 1 Effects of Recombinant Human CHI3L1 on the Levels of mRNA Encoding Human ACE2, TMPRSS2, and Ctsl in Various Lung Cells In Vitro

The coronavirus SARS-Cov-2 is known to enter cells via a cellular receptor called Angiotensin-Converting Enzyme 2 (ACE2). The viral spike or “S” proteins stick out of the SARS-Cov-2 virus and bind to ACE2. To get inside the cell, the spike protein has to be modified by enzymes called proteases including cathepsin L (CSTL). The binding of the virus's phosphorylated spike protein and ACE2 allows the virus to enter. Once in the cell, the virus replicates and subsequently released by the cell to infect other cells. In the process, it causes cell death and tissue injury, inflammation and eventually pulmonary fibrosis (scarring).

Transmembrane protease, serine 2 (TMPRSS2) is an enzyme that that belongs to the serine protease family. The encoded protein contains a type II transmembrane domain, a receptor class A domain, a scavenger receptor cysteine-rich domain and a protease domain. Serine proteases are known to be involved in many physiological and pathological processes. Some coronaviruses, e.g., both the SARS coronavirus of 2003 and the SARS-CoV-2 are activated by TMPRSS2 and can thus be inhibited by TMPRSS2 inhibitors (Hoffmann et al., March 2020).⁷⁷ SARS-CoV-2 uses the SARS-CoV receptor ACE2 for entry and the serine protease TMPRSS2 for S protein priming (Rahman et al., May 2020).⁷⁸ As such, a TMPRSS2 inhibitor might constitute a treatment option for Covid-19 infections.

The purpose of the present study was to assess the effects of recombinant human CHI3L1 on the levels of mRNA encoding ACE2, TMPRSS2, and CTSL in various types of lung cells.

### Materials and Methods

### Real Time RT-PCR

Total cellular RNA was obtained using TRIzol reagent (ThermoFisher Scientific) followed by RNA extraction using RNeasy Mini Kit (Qiagen, Germantown, MD) according to the manufacturer's instructions. mRNA was measured and used for real time (RT)-PCR as described previously.^{79,80} The primer sequences used in these studies are summarized in Table 1. Ct values of the test genes were normalized to the internal housekeeping gene β-actin.

TABLE 1
Sequences of RT-PCR primers

| Gene | | Sense (5′-3′) | Antisense (5′-3′) |
|---|---|---|---|
| Human | ACE2 | CGAAGCCGAAGACCTGTTCTA | GGGCAAGTGTGGACTGTTCC |
| | | (SEQ ID NO: 49) | (SEQ ID NO: 56) |
| | CTSL | CAAGTGCTCCAACTCTGGGAT | AACACACCGATTCTCGTCCTC |
| | | (SEQ ID NO: 50) | (SEQ ID NO: 57) |
| | TMPRSS2 | CTTTTGCCTGGGAATTGCCTC | CATCGCCTTCCACTTGGTC (SEQ |
| | | (SEQ ID NO: 51) | ID NO: 58) |
| | FURIN | CCTGGTTGCTATGGGTGGTAG | AAGTGGTAATAGTCCCCGAAGA |
| | | (SEQ ID NO: 52) | (SEQ ID NO: 59) |
| Mouse | Ace2 | TCCAGACTCCGATCATCAAGC | GCTCATGGTGTTCAGAATTGTGT |
| | | (SEQ ID NO: 53) | (SEQ ID NO: 60) |
| | Ctsl | ATCAAACCTTTAGTGCAGAGTGG | CTGTATTCCCCGTTGTGTAGC |
| | | (SEQ ID NO: 54) | (SEQ ID NO: 61) |
| | Tmprss2 | CAGTCTGAGCACATCTGTCCT | CTCGGAGCATACTGAGGCA (SEQ |
| | | (SEQ ID NO: 55) | ID NO: 62) |

### Results

### A549 Cells

A549 cells are adenocarcinomic human alveolar basal epithelial cells. In the lung tissue of their origin, alveolar basal epithelial cells are responsible for the diffusion of some substances, such as water and electrolytes, across alveoli. The cells are able to synthesize lecithin and contain high levels of unsaturated fatty acids, which are important to maintain membrane phospholipids. A549 cells are widely used as models of alveolar Type II pulmonary epithelium, finding utility in research examining the metabolic processing of lung tissue and possible mechanisms of drug delivery to the tissue.⁸¹

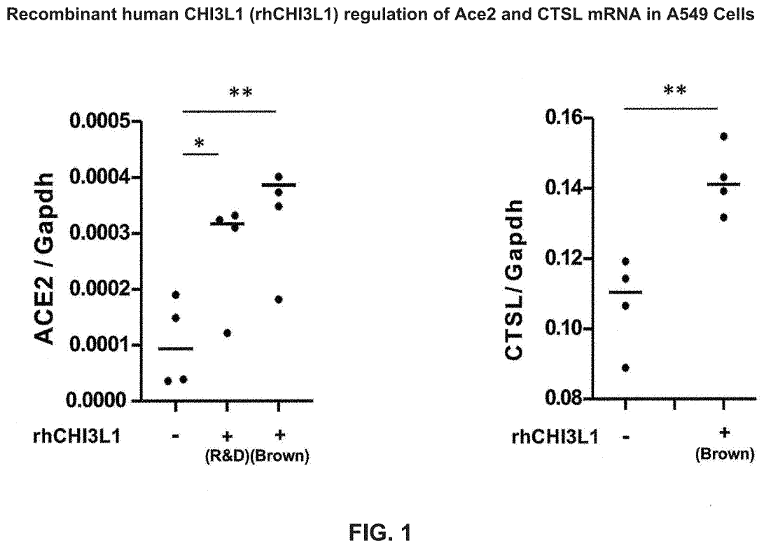

In the first study, A549 cells were incubated with rhCHI3L1 from a commercial source (R&D Inc.) and rhCHI3L1 generated at Brown University (Brown). The cells were incubated for 24 hr with stimulation of rhCHI3L1 (500 ng/mL) at 37° C. mRNA was then extracted and the levels of mRNA for ACE2 and CTSL were evaluated by real time qRT-PCR and compared to vehicle controls. Levels were expressed in relationship to GAPDH controls. As shown in FIG. 1, rhCHI3L1 significantly upregulated the levels of mRNA encoding Ace2 and cathepsin L (CTSL) in A549 cells.

In the next study, A549 cells were incubated with 0, 250 ng/mL, or 500 ng/mL of rhCHI3L1 for 24 hr with stimulation of rhCHI3L1 at 37° C. mRNA was then extracted and the levels of mRNA encoding ACE2, TMPRSS2, and CTSL were assessed via qRT-PCR. As shown in FIG. 2, rhCHI3L1 significantly upregulated the levels of mRNA encoding ACE2, TMPRSS2, and CTSL.

The observed upregulation of ACE2, TMPRSS2, and CTSL in A549 cells suggests that CHI3L1 may be a major contributor to the pathogenesis of Covid 19.

### Human Small Airway Epithelial Cells (HSAEC)

HSAEC are isolated from the distal portion of the human respiratory tract in the 1 mm bronchiole area. The distal respiratory tract mainly consists of pulmonary alveoli, which are spherical outcroppings of the respiratory bronchioles, and are the primary sites of gas exchange with the blood. HSAEC are used for functional studies to investigate disorders such as microbial (e.g., viral, bacterial) infection and pathogenesis; airway inflammation and wound healing; asthma; pulmonary fibrosis, chronic obstructive pulmonary disease; emphysema; toxicology/other testing of pharmaceuticals.⁸²

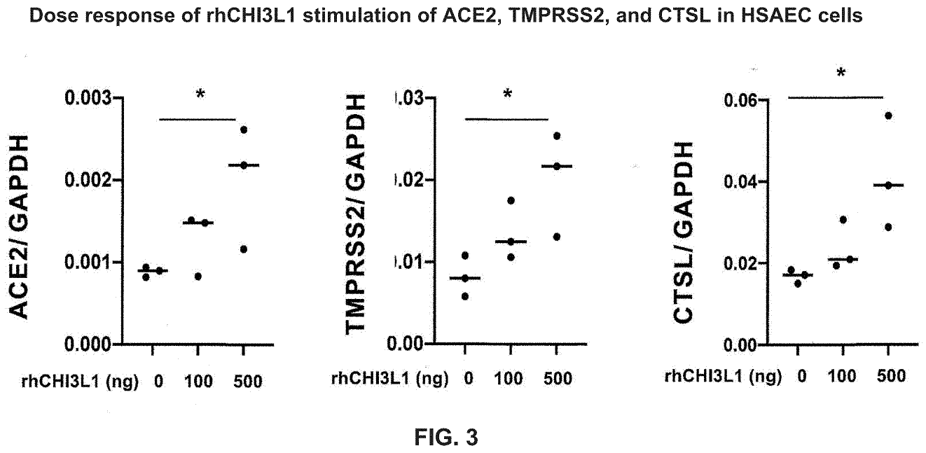

In the present study, normal HSAEC were incubated with 0, 100 ng/mL, or 500 ng/mL of rhCHI3L1 for 24 hours at 37° C. The levels of mRNA encoding ACE2, TMPRSS2, and CTSL were assessed via qRT-PCR and expressed in relationship to GAPDH controls. As shown in FIG. 3, 500 ng/mL of rhCHI3L1 significantly upregulated the levels of mRNA encoding ACE2, TMPRSS2, and CTSL.

The observed upregulation of ACE2, TMPRSS2, and CTSL in normal HSAEC suggests that CHI3L1 may be a major contributor to the pathogenesis of Covid 19.

### Calu-3 Cells

Calu-3 is a human lung cancer cell line commonly used in cancer research and drug development. Calu-3 cells are epithelial and can act as respiratory models in preclinical applications.⁸³ Calu-3 cells have been used to study SARS-CoV-2.⁸⁴

The present study not only assessed the effects of rhCHI3L1 on the levels of mRNA encoding ACE2, TMPRSS2, and CTSL in Calu-3 cells, it also assessed the effects on the levels of mRNA encoding furin. Furin, also known as PACE (Paired basic Amino acid Cleaving Enzyme), is a subtilisin-like peptidase. Some proteins are inactive when they are first synthesized and must have sections removed in order to become active. Furin cleaves these sections and activates the proteins. The spike glycoprotein of SARS-CoV-2 has recently been reported to contain a furin-like cleavage site for host cell furins that was not present in the genome sequence of other SARS-like CoVs.⁸⁵ Furthermore, the present study will also assess if the rhCHI3L1-induced upregulation of ACE2, TMPRSS2, CTSL, and FURIN can be inhibited by FRG, an anti-CHI3L1 monoclonal antibody.

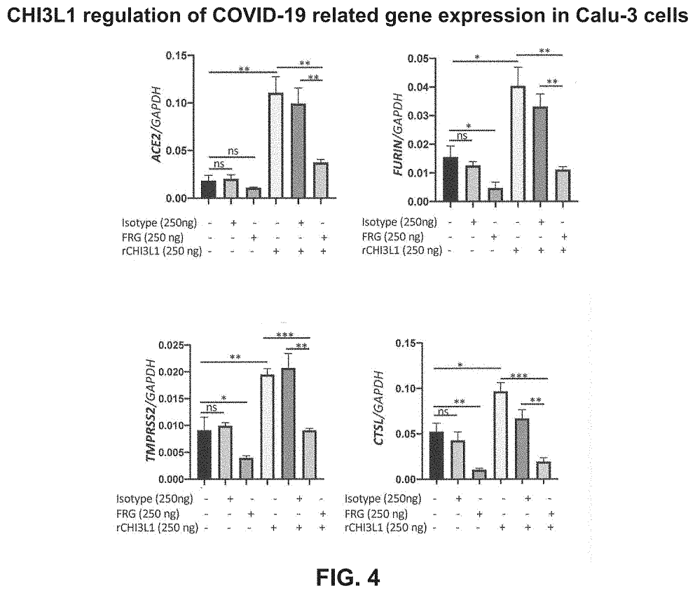

Calu-3 cells were incubated with vehicle or 250 ng/mL rhCHI3L1 for 24 hours in the presence of 250 ng/mL FRG or its isotype control (isotype) at 37° C. Levels of mRNA encoding ACE2, FURIN, TMPRSS2, and CTSL were assessed via RT-PCR. As shown in FIG. 4, rhCHI3L1 significantly upregulated the levels of mRNA encoding not only ACE2 (top left), TMPRSS2 (bottom left), and CTSL (bottom right) but also FURIN (top right) in Calu-3 lung epithelial cells. FRG modestly diminished the levels of basal ACE2 expression and potently inhibited the ability of rhCHI3L1 to stimulate ACE2 mRNA accumulation. FRG also potently decreased the basal and rhCHI3L1-stimulated expression of TMPRSS2, CTSL and FURIN.

Again, the observed upregulation of ACE2, TMPRSS2, CTSL, and FURIN in Calu-3 cells suggests that CHI3L1 may be a major contributor to the pathogenesis of Covid 19. Importantly, the anti-CHI3L1 monoclonal antibody, FRG, was able to decrease the basal and rhCHI3L1-stimulated enhancement of mRNA levels of ACE2, TMPRSS2, CTSL and FURIN. These data suggests that CHI3L1 inhibitors, such as FRG, may provide an effective treatment for Covid-19.

### Example 2 Evaluation of ACE2, TMPRSS2, and Ctsl in Wild Type and CHI3L1 Transgenic Mice

In Example 1, rhCHI3L1 was demonstrated to significantly increase the levels of mRNA encoding ACE2, TMPRSS2, CTSL and FURIN in vitro in three lung cell lines: A549 cells, HSAEC, and Calu-3 cells. To further define the role of CHI3L1 in the pathogenesis of Covid-19, CHI3L1 transgenic (Tg) mice were used, in which CHI3L1 was selectively and inducibly targeted to the lung using the CC10 promoter.⁸⁶ The present study assessed whether similar increases in mRNA levels encoding Ace2, Tmprss2, AND Ctsl would be observed in CHI3L1 transgenic (Tg) mice and if such mRNA increases results in increase expression of the encoded proteins.

### Materials and Methods

### CHI3L1 Transgenic (Tg) Mice

Tg mice in which human CHI3L1 was tightly and inducibly overexpressed (CC10-rtTA-tTS-CHI3L1) in a lung-specific manner were generated using constructs and approaches that have been previously described by our laboratory.⁸⁷ The CC10 promoter, reverse tetracycline transactivator, and tetracycline-controlled transcriptional suppressor were used to overexpress CHI3L1 in the mouse lung. These CC10-rtTA-tTS-CHI3L1 Tg mice had an appropriately targeted and inducible transgene (bronchoalveolar lavage—CHI3L1, 400-450 ng/mL after 48 h of doxycycline [dox] administration) and normal lungs on gross and light microscopic examination after 4 weeks of dox administration (not shown).

### Immunohistochemistry

Formalin-fixed paraffin embedded (FFPE) lung tissue blocks were serially sectioned at 5 μm-thickness and mounted on glass slides. After deparaffinization and dehydration, heat-induced epitope retrieval was performed by boiling the samples in a steamer for 30 minutes in antigen unmasking solution (Abcam, antigen retrieval buffer, 100× citrate buffer pH:6.0). To prevent nonspecific protein binding, all sections were blocked in a ready-to-use serum free protein blocking solution (Dako/Agilent, Santa Clara, CA) for 10 minutes at room temperature. The sections were then incubated with primary antibodies (α-Ace2 (R&D, Cat #AF3437), α-Cathepsin L (R&D, Cat #AF1515)), Tmprss2 (Abcam, Cat #92323) overnight at 4° C. After three washings, fluorescence-labeled secondary antibodies were incubated for 1 hour at room temperature. The sections were then counterstained with Blue-fluorescent DAPI (4′,6-diamidino-2-phenylindole) for nuclei stain and cover slips were added.

### Results

mRNA Levels in Mice Lungs

CHI3L1 Tg mice (n=6) were exposed to normal water or dox-treated water for 2 weeks to overexpress CHI3L1 in the mouse lung. Lungs were obtained from wild type (WT; −) and lung targeted CHI3L1 transgenic (Tg; +) mice. mRNA was extracted, and the levels of mRNA for Ace2 and Ctsl were evaluated by real-time qRT-PCR. Levels were expressed in relationship to p-actin controls. Glyceraldehyde-3-phosphate dehydrogenase (Gapdh) was used as an internal control.

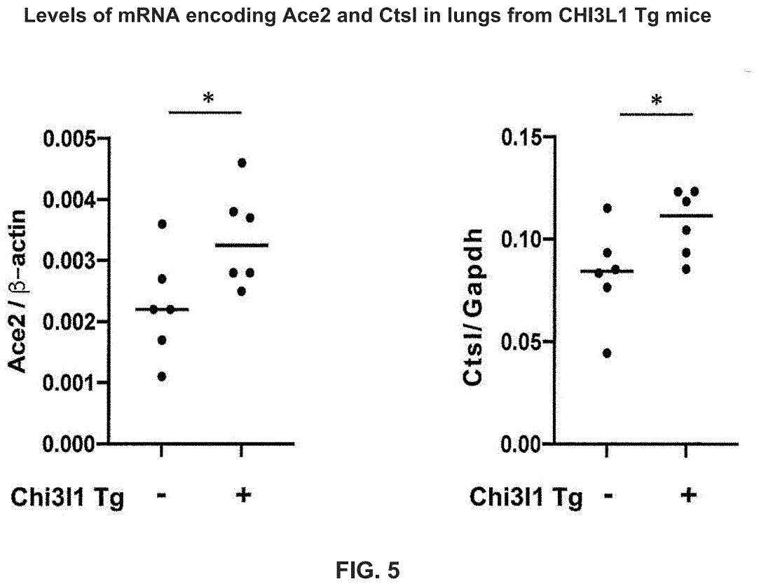

As shown in FIG. 5, the levels of mRNA encoding Ace2 and Ctsl were significantly elevated in the lungs of CHI3L1 Tg mice compared to wild type mice. These results suggest that, as was observed in vitro with lung cells, CHI3L1 can also upregulate the levels of mRNA encoding Ace2 and Ctsl in the mice lungs in vivo.

### Protein Expression in Mice Lungs

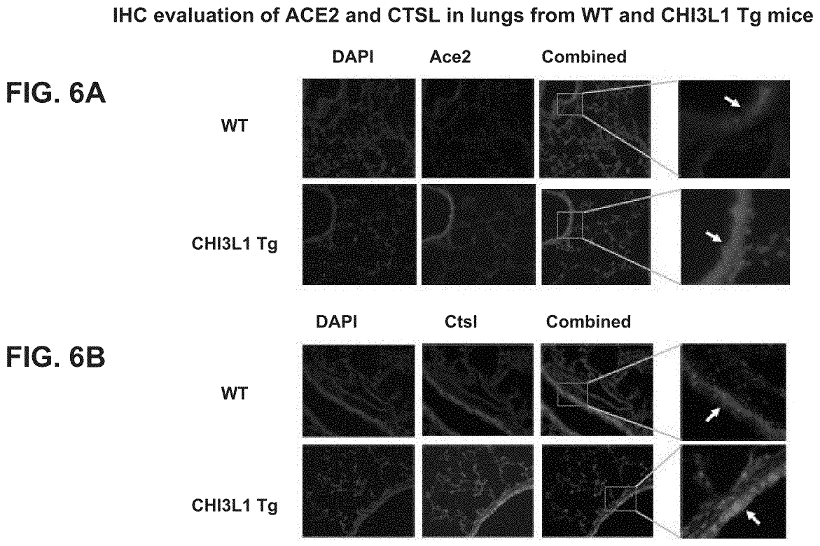

In FIG. 6A-B, lungs obtained from wild type (WT; −) and lung targeted CHI3L1 transgenic (Tg; +) mice were assessed for Ace2 and Ctsl expression. Blue-fluorescent DAPI was used for nuclei stain. Red-fluorescence (RFP) and green fluorescence (FITC)-labeled antibodies against Ace2 and Ctsl were used for detection of Ace2 and Cathepsin L expression or accumulation in the lungs, respectively.

As shown in FIG. 6A, the expression of Ace2 was much more prominent in the lungs of CHI3L1 Tg mice compared to the lungs of WT mice, especially in airway epithelial cells (right-most panels). Similarly, as shown in FIG. 6B, the expression of Cathepsin L was much more prominent in the lungs of CHI3L1 Tg mice compared to the lungs of WT mice, especially in airway epithelial cells (right-most panels).

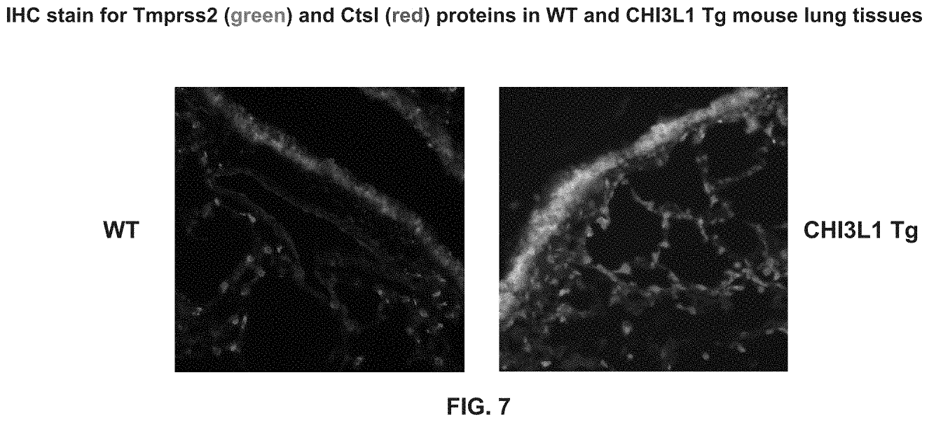

Double label immunohistochemistry (IHC) was then used to compare the accumulation of Tmprss2 and Ctsl in lungs from wild type (WT) and CHI3L1 Tg mice. Tmprss2 proteins were stained in green and Ctsl were proteins stained in red. Co-localized enzymes displayed in yellow. As shown in FIG. 7, the heightened co-localized staining of Tmprss2 and Cathepsin L can be seen in airway and, to a lesser degree, alveolar epithelial cells.

### SUMMARY

In summary, these results suggest that CHI3L1 stimulates ACE2 and a few of the spike activating proteases (SAPs), namely TMPRSS2, CTSL, and FURIN. The SAP stimulation was particularly striking for cathepsin L. These data suggested that this CHI3L1-ACE2 pathway may be a major contributor to the pathogenesis of Covid-19.

Importantly, the CHI3L1 stimulation of ACE2 and the SAPs was fully reversed by a CHI3L1 inhibitor, the anti-CHI3L1 monoclonal antibody, FRG. These data suggest that CHI3L1 inhibitors may represent a more effective therapeutic agents to treat and reverse the effects of Covid-19 infections.

### Example 3 Effects of Kasugamycin on the Levels of mRNA Encoding Human ACE2, TMPRSS2, and Ctsl in Calu-3 Lung Epithelial Cells In Vitro

Kasugamycin (KSM) is an inhibitor of CHI3L1 and chitinase 1. The present study assessed the effects of KSM on the levels of mRNA encoding ACE2, FURIN, TMPRSS2, and CTSL in Calu-3 cells.

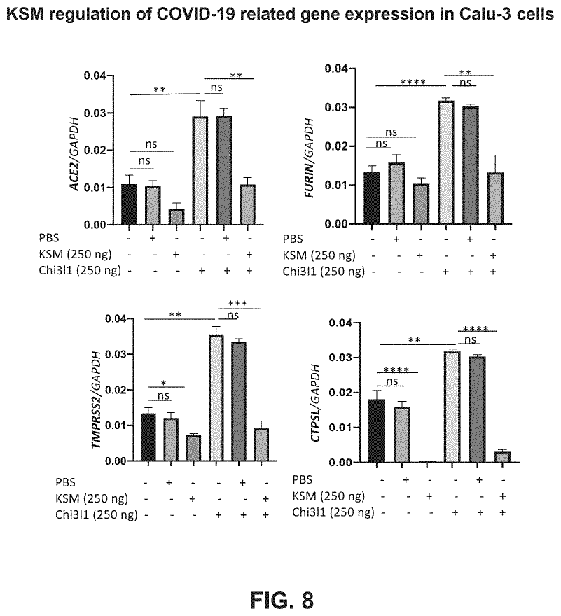

As shown in FIG. 8, CHI3L1 significantly upregulated the levels of mRNA encoding ACE2 (top left), FURIN (top right) TMPRSS2 (bottom left), and CTSL (bottom right) in Calu-3 lung epithelial cells. KSM diminished the levels of basal ACE2 expression and potently inhibited the ability of rhCHI3L1 to stimulate ACE2 mRNA accumulation. KSM also potently decreased the basal and rhCHI3L1-stimulated expression of TMPRSS2, CTSL and FURIN.

The observed upregulation of ACE2, TMPRSS2, CTSL, and FURIN in Calu-3 cells further confirms that CHI3L1 may be a major contributor to the pathogenesis of Covid 19. Importantly, KSM was able to decrease the basal and rhCHI3L1-stimulated enhancement of mRNA levels of ACE2, TMPRSS2, CTSL and FURIN. These data suggests that an inhibitor of CHI3L1 and chitinase 1, such as KSM or a derivative, analog, or variant thereof, can provide an effective treatment for Covid-19.

### Example 4 Effects of Flavopiridol on the Levels of mRNA Encoding Human ACE2, TMPRSS2, and CTSL in Calu-3 Lung Epithelial Cells In Vitro

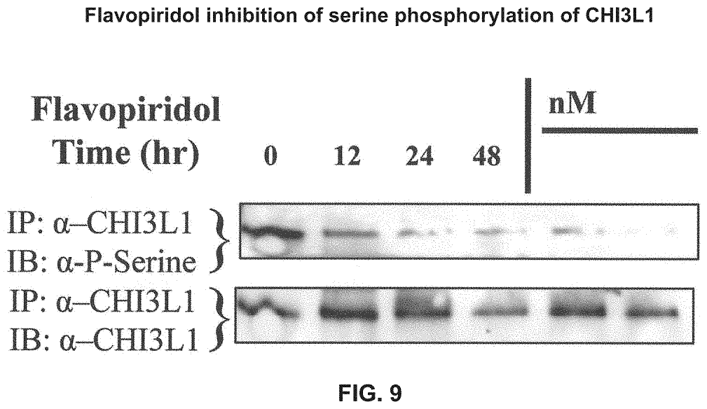

As shown in FIG. 9, the serine phosphorylation of CHI3L1 is blocked by flavopiridol, a broad spectrum CDK inhibitor, in a time-dependent and dose-dependent fashion. This represents the first demonstration that CHI3L1 is a phosphoprotein that has CDK binding site. Several other CDK inhibitors were tested (e.g., Palbociclib, Dinaciclib, Indirubin) and they showed variable degree of phosphorylation changes of CHI3L1 (data not shown). Flavopiridol, as a pan-CDK inhibitor, showed the most prominent changes in CDK phosphorylation and was used for subsequent studies.

The next study assessed whether the inhibition of CHI3L1 phosphorylation wound result in similar effects observed with CHI3L1 inhibitors on the levels of mRNA encoding ACE2 and spike activating proteases (SAPs), namely FURIN, TMPRSS2, and CTSL, in Calu-3 cells (described in Example 1).

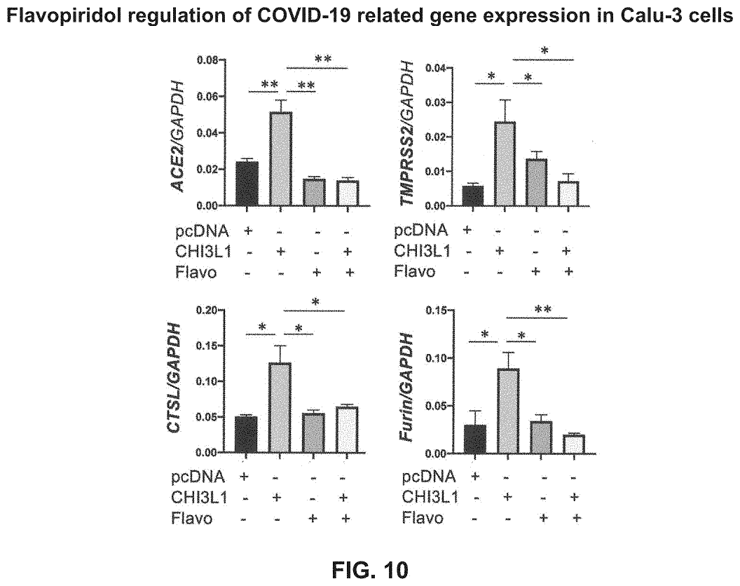

As shown in FIG. 10, CHI3L1 significantly upregulated the levels of mRNA encoding ACE2 (top left), TMPRSS2 (top right), CTSL (bottom left), and FURIN (bottom right) in Calu-3 lung epithelial cells. Flavopiridol (25 nM) diminished the levels of basal ACE2 expression and potently inhibited the ability of CHI3L1 to stimulate ACE2 mRNA accumulation. Flavopiridol also potently decreased the basal and CHI3L1-stimulated expression of the three SAPs, TMPRSS2, CTSL and FURIN.

The observed upregulation of ACE2, TMPRSS2, CTSL, and FURIN in Calu-3 cells further confirms that phosphorylated CHI3L1 may be a major contributor to the pathogenesis of Covid 19. Importantly, the inhibition of CHI3L1 phosphorylation by flavopiridol was able to decrease the basal and CHI3L1-stimulated enhancement of mRNA levels of ACE2, TMPRSS2, CTSL and FURIN. These data suggests that an inhibitor of CHI3L1 phosphorylation, such as CDK inhibitors, can provide an effective treatment for Covid-19.

### Example 5 Effects of CHI3L1 Inhibition on Covid-19 Variants

Covid-19 is caused by a highly transmissible novel coronavirus, severe acute respiratory syndrome coronavirus 2 (SARS-CoV-2). All viruses, including SARS-CoV-2, change over time through mutation. Most changes have little to no impact on the virus' properties. However, some changes do affect the virus's properties, such as how easily it spreads, the associated disease severity, or the performance of vaccines, therapeutic medicines, diagnostic tools, or other public health and social measures.

SARS-CoV-2 is an RNA virus, a family with significant adaptive evolution due to high mutation rates.⁸⁸ Although the changes in coronaviruses are slower than most RNA viruses, there are some viral components in SARS-CoV-2 that already yielded relevant mutations.^{89,90,91,92,93,94}

Using SARS-CoV-2 pseudovirus infection, the present studies assessed whether the therapeutic strategies of the present disclosure are also effective against SARS-CoV-2 mutations.

### SARS-CoV-2 Pseudovirus Infection

Pseudotyped SARS-CoV-2 virus which has a lentiviral core expressing green fluorescent protein (GFP) but with the SARS-CoV-2 spike protein (expressing D164G and E484K common variant forms of S protein) on its envelope were obtained from COBRE Center for Stem Cells and Aging established at Brown University and Rhode Island Hospital. UK (United Kingdom; B.1.1.7), SA (South African; B.1.351) and BR (Brazilian; P.1) variants of pseudovirus were purchased from BPS Bioscience (San Diego, CA). A plasmid expressing VSV-G protein instead of the S protein was used to generate a pantropic control lentivirus. Calu-3 cells were stimulated with rCHI3L1 (250 ng/mL) with and without FRG antibody or other CHI3L1 inhibitors, incubated for 24 hours, and then infected Pseudovirus. SARS-CoV-2 pseudovirus or VSV-G lentivirus were used to spin-infect Calu-3 cells in a 12-well plate (931 g for 2 hours at 30° C. in the presence of 8 μg/ml polybrene). Fluorescence microscopic images were taken 18 hours after infection. Flow cytometry analysis of GFP (+) cells was carried out 48 hours after infection on a BD LSRII flow cytometer and with the FlowJo software.

### SARS-CoV-2 Mutation Variants

The following mutation variants were used in the present study: the D614G variant; the E484K variant; the alpha or United Kingdom (UK) variant; the beta or South African (SA) variant; and the gamma or Brazilian (BZ) variant.

### Results

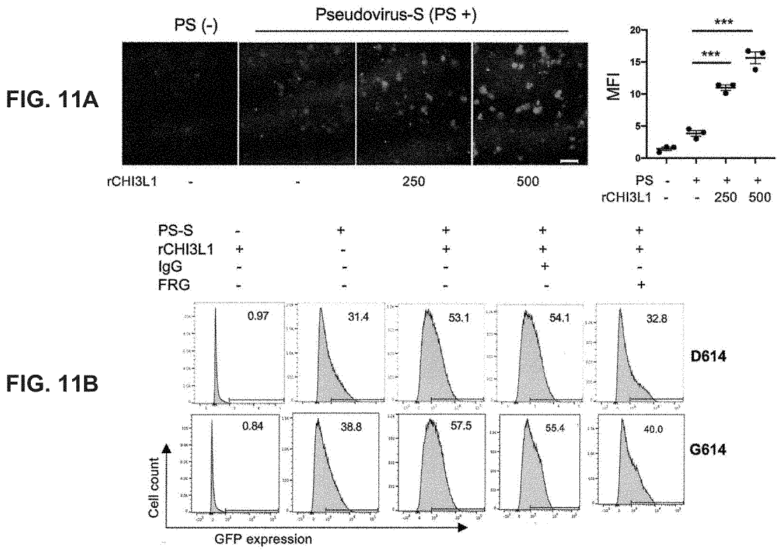

Calu-3 cells were incubated with vehicle (rCHI3L1(−)) or the noted concentrations of rCHI3L1 for 24 hours and then transfected with a pseudovirus containing the S protein (PS; D614 and G164 variants) from SC2 and a GFP expression construct. The transfected cells were incubated for additional 24 hours and evaluated using fluorescent microscopy. FIG. 11A shows the quantification of mean fluorescent intensity (MFI), as can be seen in the dot plot on the right. In FIG. 11B, Calu-3 cells were incubated with rCHI3L1 (250 ng/mL) or vehicle (PBS) for 24 hours in the presence or absence of an antibody against CHI3L1 (the FRG antibody) or control antibody (IgG). The Calu-3 cells were infected with spike protein (S)-containing pseudovirus (PS-S; D614 and G614 variants) expressing GFP and GFP expression was evaluated by flow cytometry. CHI3L1 stimulated cellular integration of S proteins in D614 and G614 variants and FRG abrogated the CHI3L1 effect.

The following variant forms of the Spike proteins were then assessed: D614G, E484K, United Kingdom (UK strain), South African (SA), and Brazilian (BZ). Calu-3 cells were incubated with either the vehicle (PBS), a control antibody (IgG), FRG (an anti-CHI3L1 antibody), or Kasugamycin (KSM) with or without stimulation of recombinant CHI3L1 (rCHI3L1; 250 ng/mL) for 24 hours. They were then transfected with a pseudovirus (PS) containing the various mutations of S protein (D614G, E484K, United Kingdom (UK strain), South African (SA). Brazilian (BZ) from SC2 and a GFP expression construct. The transfected cells were incubated for additional 48 hours and then evaluated by FACS analysis.

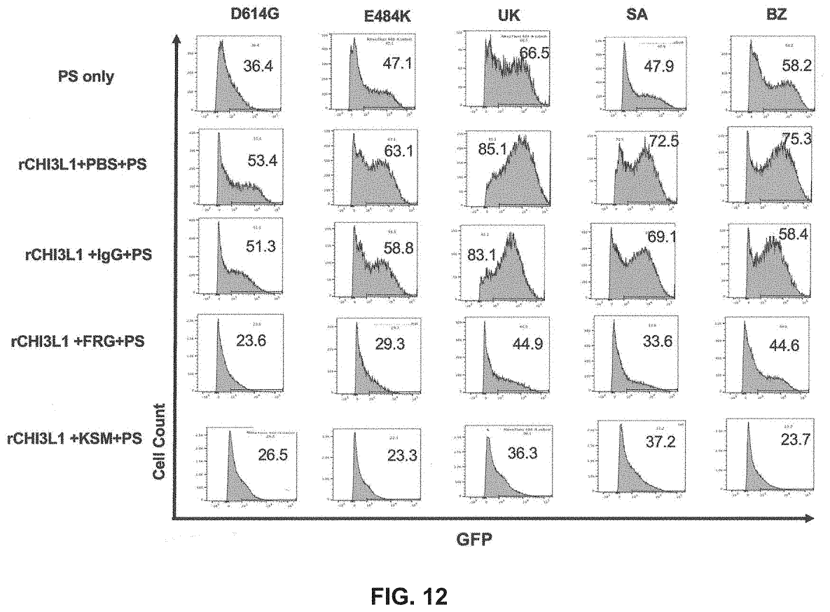

As shown in FIG. 12, CHI3L1 stimulated cellular integration of Spike proteins of SARS-Cov2 and CHI3L1 inhibitors abrogated the CHI3L1-stimulated pseudoviral infection effect of all variant forms of S proteins tested.

These data suggests that CHI3L1 inhibition provides an effective therapeutic strategy for variants of SARS-CoV-2 and can be used for the prevention, reversal, and/or treatment of Covid-19 infections.

### Example 6 Effects of CHI3L1 Inhibition on Covid-19 Delta Variants

First identified in India in December 2020, the delta variant swept rapidly through that country and Great Britain before reaching the U.S., where it quickly surged. Delta is believed to be more than twice as contagious as previous variants, and studies have shown that it may be more likely than the original virus to put infected people in the hospital. In two different studies from Canada and Scotland, patients infected with the delta variant were more likely to be hospitalized than patients infected with alpha or the original virus strains.^{95,96} Some data suggest the delta variant causes more severe illness than previous strains in unvaccinated persons.^{97,98} The delta variant is now the predominant SARS CoV-2 variant, accounting for more than 99% of COVID-19 cases in the U.S. and leading to an overwhelming increase in hospitalizations in some states.

Using SARS-CoV-2 pseudovirus infection, the present studies assessed whether the therapeutic strategies of the present disclosure are also effective against the delta variant and other SARS-CoV-2 mutations.

### Materials and Methods

Generation of Pseudoviruses with S Protein Mutations

To determine if the S protein mutations that are prominent features of SARS CoV-2 variants cause alterations in the ability of the virus to infect epithelial cells, pseudoviruses were generated with wild type S proteins or the S mutations that are seen in the alpha, beta, gamma and delta variants. These pseudotyped SARS-CoV-2 virus moieties had a lentiviral core expressing green fluorescent protein (GFP) and the SARS-CoV-2 spike protein but lacked core SARS CoV-2 sequences. Pseudotyped virus with mutated S and ancestral S proteins were compared in their ability to infect untreated and or treated Calu-3 epithelial cells. The mutations that were used to simulate each of the major mutants can be seen in Table 5 below.

TABLE 5

| Alpha (α) UK-VARIANT | Beta (β) South Africa SA-VARIANT |
|---|---|
| (Spike Mutations in B.1.1.7 variant) | (Spike Mutations in the B.1.351 variant) |
| Deletions of H69, V70, and Y144; | L18F |
| N501Y | D80A |
| A570D | D215G |
| D614G | R246I |
| P681H | K417N |
| T716I | E484K |
| S982A | N501Y |
| D1118H | D614G |
| Source: B.1.1.7: BPS BIOSCIENCE#78112-1 | A701V |
| | Source: B.1.351: BPS BIOSCIENCE#78142-1 |
| Gamma (γ) Brazil BZ-VARIANT | Delta (δ) India IN-VARIANT |
| (Spike Mutations in P.1 variant) | Spike Mutations in B.1.617.2 Variant (Delta T19R) |
| L18F | G142D |
| T20N | 156/157 DELETION |
| P26S | R158G |
| D138Y | L452R |
| R190S | T478K |
| K417T | D614G |
| E484K | P681R |
| N501Y | D950N |
| D614G | Source: BPS bioscience# 78216-1 |
| H655Y | |
| T1027I | |
| Source: P.1: BPS BIOSCIENCE#78144-1 | |

A plasmid expressing VSV-G protein instead of the S protein was used to generate a pantropic control lentivirus. Calu-3 cells were stimulated with rCHI3L1 (250 ng/mL) with and without FRG antibody or other CHI3L1 inhibitors, incubated for 24 hours, and then infected Pseudovirus. SARS-CoV-2 pseudovirus or VSV-G lentivirus were used to infect Calu-3 cells in a 12-well plate (931 g for 2 hours at 30° C. in the presence of 8 μg/ml polybrene). Fluorescence microscopic images were taken 18 hours after infection. Fluorescence activated cell sorting (FACS) analysis of GFP (+) cells was carried out 72 hours after infection.

### Effects of CHI3L1 on Epithelial Cell Uptake

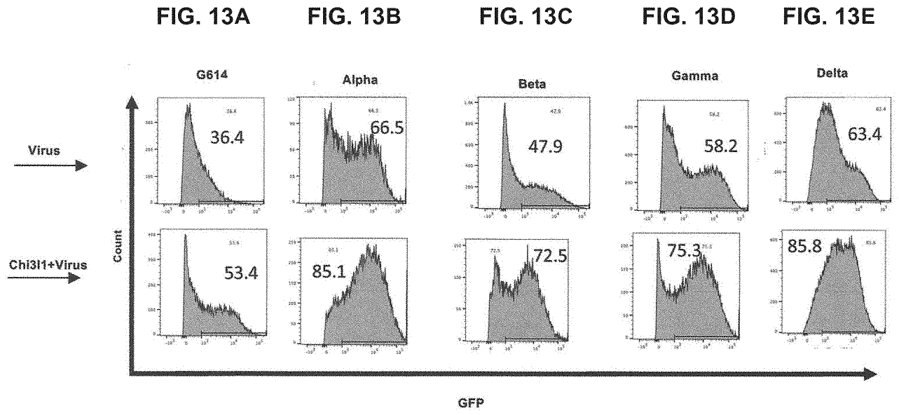

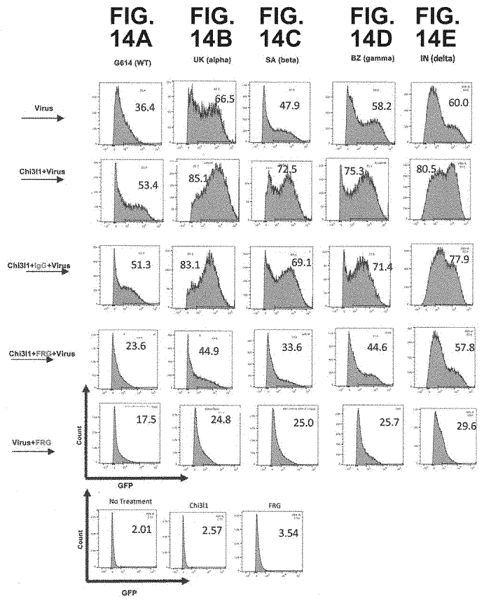

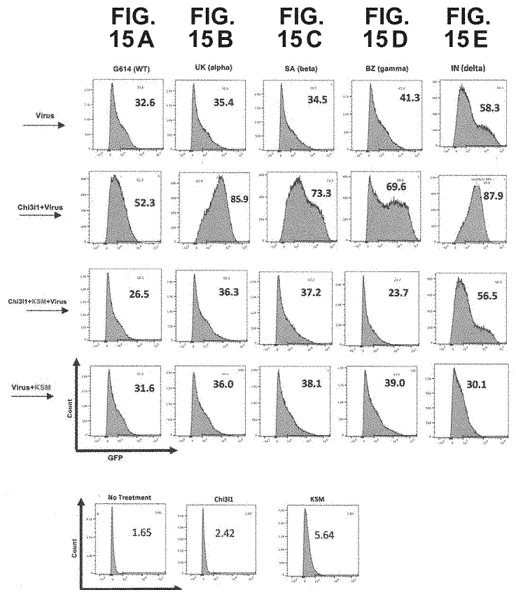

As demonstrated above, CHI3L1 is a potent stimulator of epithelial expression of ACE2, TMPRSS2, and CTSL and epithelial cell viral uptake. To determine if the major S protein variants altered these responses, the uptake of pseudovirus with ancestral and mutated S proteins by untreated and CHI3L1-treated Calu-3 cells were compared. As can be seen in FIG. 13A, CHI3L1 was a potent stimulator of the uptake of pseudovirus with the ancestral G614 S protein. Similar increases in Calu-3 cell pseudovirus uptake were seen when the S proteins that are characteristic of the alpha, beta or gamma variants were employed (FIG. 13A-D). When viewed in combination, these studies demonstrate that CHI3L1 augments SARS-CoV-2 pseudovirus uptake when the ancestral G614D and alpha, beta, or gamma S protein mutations are present.

### Effects of CHI3L1 on Epithelial Cell Uptake of Delta Pseudotyped SC2 Viruses

Because the Delta SARS-CoV-2 variant manifests enhanced viral infectivity and has spread widely since it first appeared in December 2020, the capability of CHI3L1 to alter the ability of the Delta variant to infect human epithelial cells was assessed. As shown in FIG. 13E, CHI3L1 was a potent stimulator of the uptake of pseudovirus with Delta S protein mutations.

The findings noted above and these observations, in combination, demonstrate that CHI3L1 is a stimulator of human epithelial cell uptake of SC2 viral pseudotypes with S protein mutations from all major SARS-CoV-2 variants of concern.

### Effects of FRG on CHI3L1-Induced Increase in Epithelial Cell Uptake of α, β, and γ Variants

These studies were undertaken to assess the effects of the monoclonal anti-CHI3L1 antibody, FRG, on the uptake of pseudovirus by Calu-3 cells treated with and without CHI3L1. As can be seen with the ancestral G614 S protein mutation, treatment of Calu-3 cells with rCHI3L1 augmented pseudovirus uptake and FRG abrogated this increase while treatment with the IgG control did not (FIG. 14A). Interestingly, FRG also diminished pseudovirus uptake by Calu-3 cells even when exogenous CHI3L1 was not administered (FIG. 14A). rCHI3L1 had similar stimulatory effects in experiments using pseudovirus with the alpha, beta, and gamma S protein mutations (FIG. 14B-D). Importantly, the uptake of pseudovirus with each of the S mutations in cells treated with and without rCHI3L1 was markedly diminished by FRG as well (FIG. 14A-D).

When viewed in combination, these studies demonstrate that monoclonal anti-Chi311 targeting exogenous and or endogenous CHI3L1 effectively inhibits the uptake of pseudovirus with ancestral, alpha, beta or gamma S protein mutations.

### Effects of FRG on CHI3L1-Induced Increase in Epithelial Cell Uptake of δ Variant

Because the Delta SARS-CoV-2 variant had such devastating clinical infectivity, FRG was also assessed for its ability to alter ability to infect human epithelial cells. As shown in FIG. 14E, CHI3L1 was a potent stimulator of the uptake of pseudovirus with Delta S protein mutations and FRG abrogated this increase while treatment with the IgG control did not. Interestingly, FRG also diminished pseudovirus uptake by Calu-3 cells even when exogenous CHI3L1 was not administered (FIG. 14E).

When viewed in combination, these studies demonstrate that monoclonal anti-CHI3L1 targeting exogenous and or endogenous CHI3L1 effectively inhibits the uptake of pseudovirus with the Delta or the other S protein mutations in the major SARS-CoV-2 viruses of concern (VOC).

### Effects of KSM on CHI3L1-Induced Increase in Epithelial Cell Uptake of α, β, and γ Variants

Previous studies from our laboratory demonstrated that kasugamycin (KSM) is a potent inhibitor of CHI3L1. In accord with these findings, these studies were undertaken to define the effects of KSM on the uptake of pseudovirus by Calu-3 cells treated with or without CHI3L1. As can be seen with the ancestral G614 S protein mutation, treatment of Calu-3 cells with rCHI3L1 augmented pseudovirus uptake and KSM abrogated this stimulatory effect (see FIG. 15A). Interestingly, KSM diminished pseudovirus uptake by Calu-3 cells even when exogenous rCHI3L1 was not administered (FIG. 15A). rCHI3L1 had similar stimulatory effects in experiments using pseudovirus with alpha, beta or gamma S protein mutations (see FIG. 15B-D). Importantly, the uptake of pseudovirus with each of the S mutations in cells treated with and without rCHI3L1 was also markedly diminished by KSM (FIG. 15B-D).

When viewed in combination, these studies demonstrate that KSM targeting exogenous and or endogenous CHI3L1 effectively inhibits the uptake of pseudovirus with ancestral, alpha, beta or gamma S protein mutations.

### Effects of KSM on CHI3L1-Induced Increase in Epithelial Cell Uptake of δ Variant

Because the Delta SARS-CoV-2 variant had such devastating clinical infectivity, KSM was also assessed for its ability to alter ability to infect human epithelial cells. As shown in FIG. 15E, CHI3L1 was a potent stimulator of the uptake of pseudovirus with delta S protein mutations and KSM abrogated this increase while treatment with the vehicle control did not. Interestingly, KSM also diminished pseudovirus uptake by Calu-3 cells even when exogenous CHI3L1 was not administered (FIG. 15E).

When viewed in combination, these studies demonstrate that KSM targeting exogenous and or endogenous CHI3L1 effectively inhibits the uptake of pseudovirus with the Delta or the other S protein mutations in the major SARS-CoV-2 viruses of concern (VOC).

### Example 7 Effects of CHI3L1 Inhibition on Covid-19 Omicron Variant

On Nov. 26, 2021, the World Health Organization (WHO) classified a new variant, B.1.1.529, as a Variant of Concern and named it omicron and on Nov. 30, 2021, the United States also classified it as a Variant of Concern (VOC).

Coronavirus disease 2019 (COVID 19), the illness caused by severe acute respiratory syndrome coronavirus virus-2 (SARS-CoV-2; SC2), was first discovered in man in 2019 and declared a global pandemic by the World Health Organization (WHO) on Mar. 11, 2020. It is the cause of a global health crisis with countries experiencing multiple waves of illness resulting in more than 273 million confirmed clinical cases and more than 5.34 million deaths as of Dec. 17, 2021.99 The disease caused by SC2 was initially noted to manifest as a pneumonia.¹⁰⁰ It is now known to have impressive extrapulmonary manifestations and vary in severity from asymptomatic to mildly symptomatic to severe disease with organ failure to death.¹⁰¹ However, the cellular and molecular events that account for the multiple waves of disease and the impressive clinical and pathologic heterogeneity that have been seen have not been defined.

SC2 interacts with cells via its spike (S) protein which binds to its cellular receptor angiotensin converting enzyme 2 (ACE2).¹⁰² To mediate viral entry the S protein is processed into S1 and S2 subunits by the S priming proteases (SPP) including TMPRSS2, cathepsin L (CTSL) and to a lesser degree, Furin. The S2 subunit mediates the fusion of the viral envelope and cell plasma membrane to allow for virus-cell entry.¹⁰³ In keeping with the importance of virus-cell interactions, many treatments for COVID 19 have focused on disease prevention using non-pharmacologic public health measures, antiviral antibodies and a critical global vaccination strategy. Treatments of acute infection include supportive interventions, anti-inflammatories, recently described oral antivirals and direct antivirals such as remdesivir.¹⁰⁴ Surprisingly, although ACE2 and SPP play critical roles in SC2 infection and proliferation, therapeutics that focus on these host moieties have not been adequately investigated.

Early strains of SC2 from Wuhan China manifest limited genetic diversity.¹⁰⁵ However, genetic epidemiologic evidence in February 2020 demonstrated the global emergence of a new dominant SC2 variant called D614G.¹⁰⁶ This variant was associated with enhanced transmissibility based on a S protein that is more likely to assume an “open” configuration and bind ACE2 with enhanced avidity when compared to the ancestral strain.¹⁰⁷ In the interval, since then multiple other SC2 variants have been appreciated. Many are now defined as variants of concern (VOC) due to their enhanced transmissibility, decreased susceptibility to neutralization by antibodies obtained from natural infection or vaccination, ability to evade detection or ability to decrease therapeutic or vaccine effectiveness.¹⁰⁸ As of June 2021, four variants (alpha, beta, gamma and delta) had been defined as VOC. Most recently omicron has been added to the list of VOC. Of these viral moieties, delta and omicron (B.1.1.529) are most problematic. The delta variant has caused the deadly second wave of disease in India and waves of COVID 19 at other sites around the world.¹⁰⁹ It is also known to have a high level of transmissibility and virulence when compared to ancestral controls and the alpha (a), beta (b), and gamma (g) variants. It manifests an enhanced ability to replicate and accumulates at very high levels in airways and tissues.¹¹⁰ In keeping with these characteristics, recent studies have also demonstrated that delta is associated with breakthrough infections in vaccinated individuals and a decrease in vaccine effectiveness, especially in the elderly.¹¹¹ The omicron variant was first detected in November 2021 and quickly declared a VOC based on its impressive transmissibility.¹¹² It has 37 S protein mutations in its predominant haplotype, 15 of which are in its receptor binding domain (RBD) which is the major target of neutralizing antibodies.¹¹³ Although it appears to cause less severe disease than delta, its impressive ability to spread and resist antibody neutralization has resulted in surges that run the risk of overwhelming health care systems worldwide. In spite of the important differences in the S proteins of the variants and the impressive importance of S and ACE2 in COVID 19, therapies that focus on host targets such as CHI3L1, ACE2 and SPP that are effective in multiple SC2 variants have not been adequately defined.

In order to assess that therapies that target the host factors involved in SC2 infection like CHI3L1 can contribute to the control of all infections induced by a virus that enters host cells via the cellular receptor ACE2, pseudoviruses that expressed S proteins from the α, β, γ, δ, and ∘ variants were employed and the ability of CHI3L1-based interventions to modify their ability to infect human lung epithelial cells were assessed.

As described below, these studies demonstrated that CHI3L1 augments the expression and accumulation of ACE2 and SPP and augments epithelial infection by the α, β, γ, δ, and ∘ pseudovirus variants. These studies also demonstrated that anti-CHI3L1 and the small molecule CHI3L1 inhibitor kasugamycin both inhibit the expression and accumulation of epithelial ACE2 and SPP and, in turn, inhibit epithelial infection by pseudoviruses that contain the α, β, γ, δ, and ∘ S proteins.

### Materials and Methods

### Cell Lines and Primary Cells in Culture

Calu-3 (HTB-55) lung epithelial cells were purchased from American Tissue Type Collection (ATCC) and maintained at 37° C. in Dulbecco's modified eagle medium (DMEM) supplemented with high glucose, L-Glutamine, minimal essential media (MEM) non-essential amino acids, penicillin/streptomycin and 10% fetal bovine serum (FBS) until used.

### Generation of Monoclonal Antibodies Against CHI3L1 (FRG)

The murine monoclonal anti-CHI3L1 antibody (FRG) was generated using peptide antigen (amino acid 223-234 of human CHI3L1) as immunogen through Abmart Inc (Berkeley Heights, NJ). This monoclonal antibody specifically detects both human and mouse CHI3L1 with high affinity (K_{d}»1.1×10⁻⁹). HEK-293T cells were transfected with the FRG construct using Lipofectamine™ 3000 (Invitrogen, #L3000015). Supernatant was collected for 7 days, and the antibody was purified using a protein A column (ThermoFisher scientific, #89960). Ligand binding affinity and sensitivity were assessed using ELISA techniques.

Infection of Pseudoviruses with S Protein Mutations

Pseudoviruses with wild type S proteins or the S mutations that are seen in the alpha, beta, gamma, and delta variants were purchased from BPS Bioscience Inc (San Diego. CA). The pseudovirus with omicron S protein was obtained from eEnzyme (Gaithersburg, MD, USA). Pseudoviruses containing S protein mutations of COVID variants used in this study can be seen in Table 6. These pseudotyped SARS-CoV-2 virus moieties had a lentiviral core expressing green fluorescent protein (GFP) and the SARS-CoV-2 spike protein but lacked core SC2 sequences. We then compared the ability of pseudotyped virus with mutated S and ancestral S proteins to infect untreated and or treated Calu-3 epithelial cells. A plasmid expressing VSV-G protein instead of the S protein was used to generate a pantropic control lentivirus. SARS-CoV-2 pseudovirus or VSV-G lentivirus were used to spin-infect Calu-3 cells in a 12-well plate (931 g for 2 hours at 30° C. in the presence of 8 μg/ml polybrene). Flow cytometry analysis of GFP (+) cells was carried out 48 h after infection on a BD LSRII flow cytometer and analyzed with the FlowJo software.

TABLE 6
Pseudoviruses containing S protein mutations of COVID variants used in this study

| Variant Name | Mutations in S protein | Pseudovirus Source (Cat #) |
|---|---|---|
| Alpha (B.1.1.7 and Q lineages) | Deletions of H69, V70, and Y144; N501Y, | BPS BIOSCIENCE Inc. |
| | A570D, D614G, P681H T716I, S982A, | Cat#78112-1 |
| | D1118H | |
| Beta (B.1.351 and | L18F, D80A, D215G, R246I, K417N, E484K, | BPS BIOSCIENCE Inc, |
| descendent lineages) | N501Y, D614G A701V | Cat#78142-1 |
| Gamma (P.1 and | L18F, T20N, P26S, D138Y, R190S K417T, | BPS BIOSCIENCE Inc. |
| descendent lineages) | E484K, N501Y, D614G H655Y, T1027I | Cart#78144-1 |
| Delta (B.1.617.2 and AY | T19R, G142D, 156/157 Deletion, R158G, | BPS Bioscience Inc. |
| lineages) | L452R, T478K D614G, P681R, D950N | Cat#78216-1 |
| Omicron (B.1.1.529 and | S371L, G339D, S375F, S373P, K417N, | eEnzyme.com |
| PANGO lineages) | N440K, G446S, S477N, T478K, E484A, | SCV2-PsV--Omicron |
| | Q498R, H505Y, N501Y, Q493R | |

### Immunofluorescence Assay (Immunocytochemistry)

Immunofluorescent staining was used to assess cellular integration of pseudoviruses associated with expression of ACE2. Briefly, Calu-3 cells were cultured in 4 well chamber slides (10⁶ cell/well) for 24 hr then infected with control and pseudoviruses for 48 hrs. Then the cells on the slides were fixed, permeabilized, and treated with blocking buffer then incubated with anti-ACE2 antibody (R&D, AF933) for overnight at 4° C. The photographs of cellular immunofluorescence of GFP (+) pseudovirus and Cy-5 (+) ACE2 expression was taken with fluorescent microscopes.

### Western Blotting (Immunoblotting)

25 μg lung or cell lysates were subjected to immunoblot analysis using antibodies against phosphorylated (p) ERK (pERK), total ERK(ERK), Phosphorylated(p) AKT (pAKT), total AKT(AKT) (Cell Signaling Tech, MA, USA). These samples were gel fractionated, transferred to membranes, and evaluated as described previously by our laboratory.¹¹⁴

### Results

### CHI3L1 Stimulates Epithelial Cell Uptake of the G614 and the Alpha, Beta, and Gamma Pseudotyped SC2 Viruses

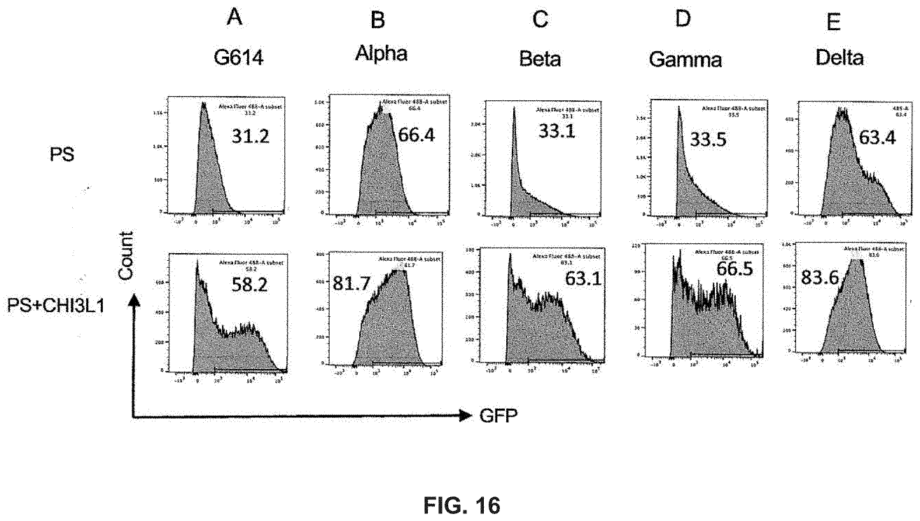

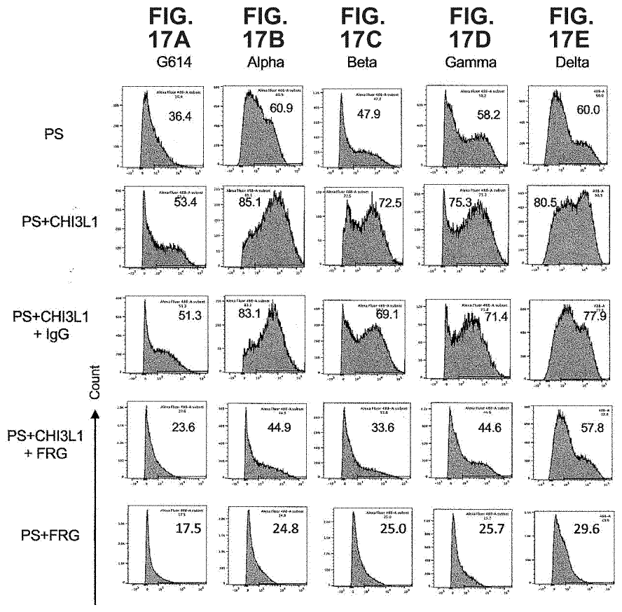

Previous studies from our laboratory demonstrated that CHI3L1 is a potent stimulator of epithelial expression of ACE2 and SPP and epithelial cell viral uptake.¹¹⁵ To determine if the major S variants altered these responses, the uptake of pseudovirus were compared with ancestral and mutated S proteins by untreated and CHI3L1-treated Calu-3 cells. As can be seen in FIG. 16A, CHI3L1 was a potent stimulator of the uptake of pseudovirus with the ancestral G614 S protein. Similar increases in Calu-3 cell pseudovirus uptake were seen when the S proteins that are characteristic of the α, β, or γ variants were employed (FIG. 16B-D). When viewed in combination, these studies demonstrate that CHI3L1 augments SC2 pseudovirus uptake when the ancestral D614G and α, β, or γ S protein mutations are present.

### CHI3L1 Stimulates Epithelial Cell Uptake of Delta Pseudotyped SC2 Viruses

Because the delta SC2 variant manifests enhanced viral infectivity and has spread widely since it first appeared in December 2020, the ability of CHI3L1 to alter its ability to infect human epithelial cells was also assessed. In these experiments, CHI3L1 was also a potent stimulator of the uptake of pseudovirus with delta S protein mutations (FIG. 16E). The findings noted above and these observations, in combination, demonstrate that CHI3L1 is a stimulator of human epithelial cell uptake of SC2 viral pseudotypes with S protein mutations from the α, β, γ, and δ VOC.

### The Monoclonal Antibody “FRG” Abrogates the CHI3L1-Induced Increase in Epithelial Cell Uptake of the G614 and the Alpha, Beta and Gamma Pseudotyped Viral Variants

Studies were next undertaken to define the effects of the monoclonal anti-CHI3L1 antibody entitled “FRG” on the uptake of pseudovirus by Calu-3 cells treated with and without CHI3L1. As was seen with the ancestral G614 S protein mutation, treatment of Calu-3 cells with rCHI3L1 augmented pseudovirus uptake and FRG abrogated this increase while treatment with the IgG control did not (FIG. 17A). Interestingly, FRG also diminished pseudovirus uptake by Calu-3 cells even when exogenous rCHI3L1 was not administered (FIG. 17A). rCHI3L1 had similar stimulatory effects in experiments using pseudovirus with a, b, or g S protein mutations (FIG. 17B-D). Importantly, the uptake of pseudoviruses with each of the S mutations in cells treated with and without rCHI3L1 was markedly diminished by FRG as well (FIG. 17A-D). When viewed in combination, these studies demonstrate that monoclonal anti-Chi311 targeting exogenous and or endogenous CHI3L1 effectively inhibits the uptake of pseudovirus with ancestral, α, β, or γ S protein mutations.

### The Monoclonal Antibody “FRG” Abrogates the CHI3L1-Induced Increase in Epithelial Cell Uptake of the Delta Pseudotyped Viral Variants

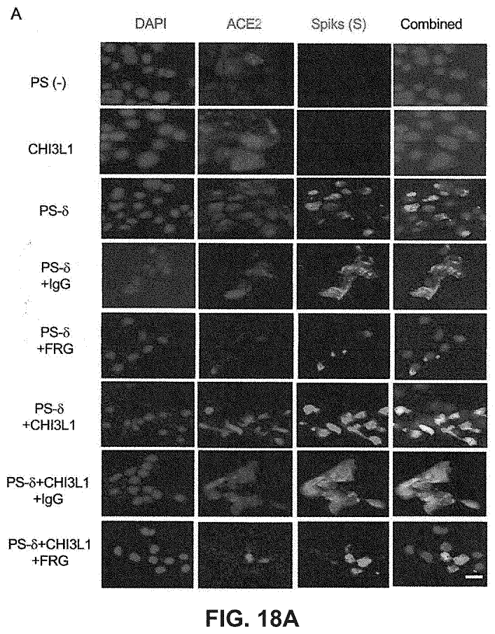

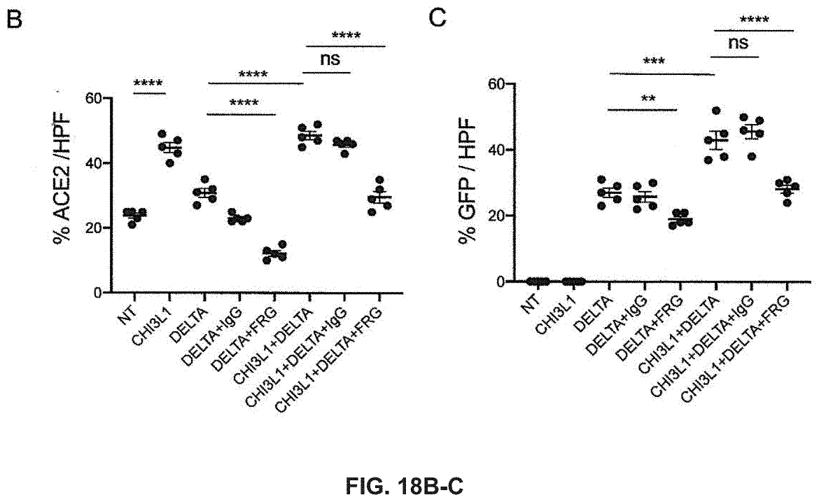

Because the delta SC2 variant has had such impressive clinical effects, the ability of FRG to alter its ability to infect human epithelial cells was also assessed. FACS-based evaluations demonstrated that CHI3L1 was a potent stimulator of the uptake of the pseudovirus with delta S protein mutations (FIG. 17E). FRG abrogated this increase while treatment with the IgG control did not (FIG. 17E). Interestingly, FRG also diminished pseudovirus uptake by Calu-3 cells even when exogenous CHI3L1 was not administered (FIG. 17E). These findings were reinforced by immunocyotchemical evaluations. These studies demonstrated that CHI3L1 augmented Calu-3 cell ACE2 accumulation and delta pseudovirus infection (FIG. 18A-C). They also demonstrated that FRG abrogated the expression of ACE2 and delta pseudovirus infection at baseline and or after the administration of rCHI3L1 (FIG. 18A-C). When viewed in combination, these studies demonstrate that monoclonal anti-CHI3L1 antibody (FRG) targeting exogenous and or endogenous CHI3L1 effectively inhibits the expression of ACE2 and the uptake of pseudoviruses with the delta or other S protein mutations.

Kasugamycin is a Small Molecule with Strong Anti-CHI3L1 Activity

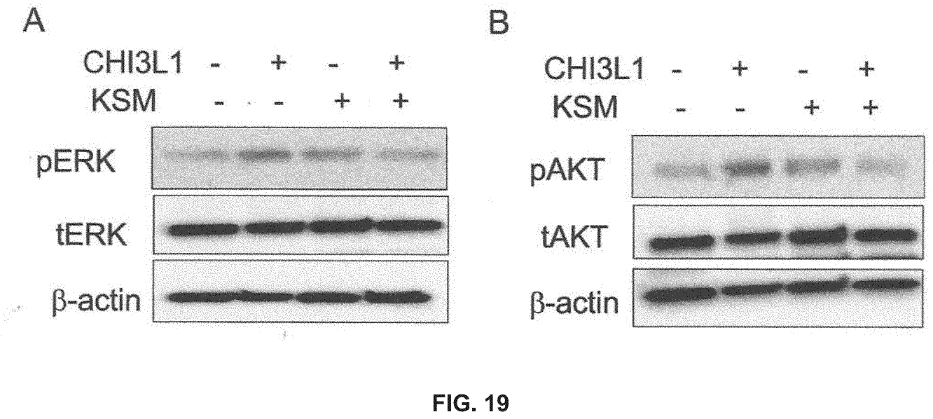

Recent studies from our lab and others identified that Kasugamycin, an aminoglycoside antibiotic, is a novel small molecule that has a strong anti-Chitinase 1 (CHIT1) activities.¹¹⁶ Since CHIT1 and CHI3L1 share structural homologies as members of 18-glycohydrolase family, we tested whether Kasugamycin (KSM) inhibits CHI3L1 activity similarly to CHIT1. As shown in FIG. 19, KSM treatment abrogated CHI3L1 stimulated MAPK and AKT activation in Calu-3 cells, suggesting a strong anti-CHI3L1 activity of KSM.

### Kasugamycin Abrogates the CHI3L1-Induced Increase in Epithelial Cell Uptake of the Alpha, Beta, and Gamma Pseudovirus Variants

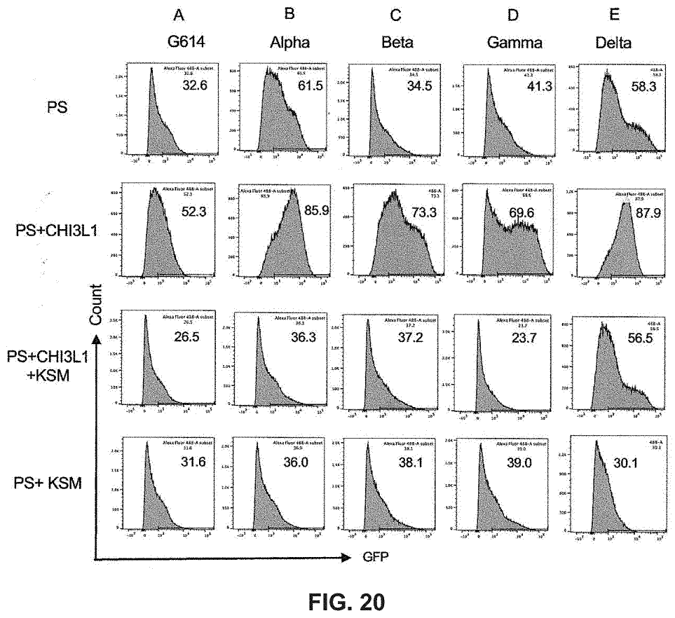

Studies were next undertaken to define the effects of kasugamycin on the uptake of pseudovirus by Calu-3 cells treated with or without rCHI3L1. As seen with the ancestral G614 S protein mutation, treatment of Calu-3 cells with rCHI3L1 augmented ancestral pseudovirus uptake and kasugamycin abrogated these stimulatory effects (FIG. 20A). rCHI3L1 had similar stimulatory effects in experiments using pseudoviruses with α, β, or γ S protein mutations (FIG. 20B-D) and these stimulatory effects were markedly decreased by kasugamycin (FIG. 20A-D). When viewed in combination, these studies demonstrate that kasugamycin targeting exogenous and or endogenous CHI3L1 effectively inhibits the uptake of pseudovirus with ancestral, α, β, or γ S protein mutations.

### Kasugamycin Abrogates the CHI3L1-Induced Increase in Epithelial Cell Uptake of Delta Pseudotyped Viral Variants

Because of the importance of the delta SC2 viral variant, the ability of kasugamycin to alter the variant's ability to infect human epithelial cells was also assessed. CHI3L1 was a potent stimulator of the uptake of pseudoviruses that contain the delta S protein mutations (FIG. 20E). Kasugamycin abrogated this increase while treatment with the vehicle control did not (FIG. 20E). Kasugamycin also diminished pseudovirus uptake by Calu-3 cells even when exogenous CHI3L1 was not administered (FIG. 20E).

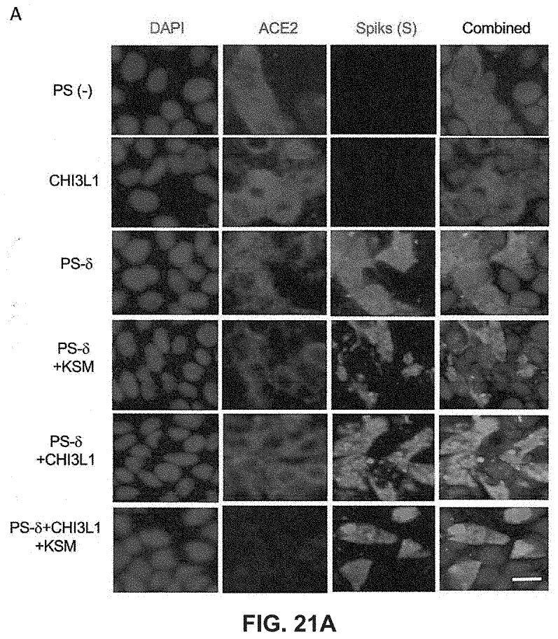

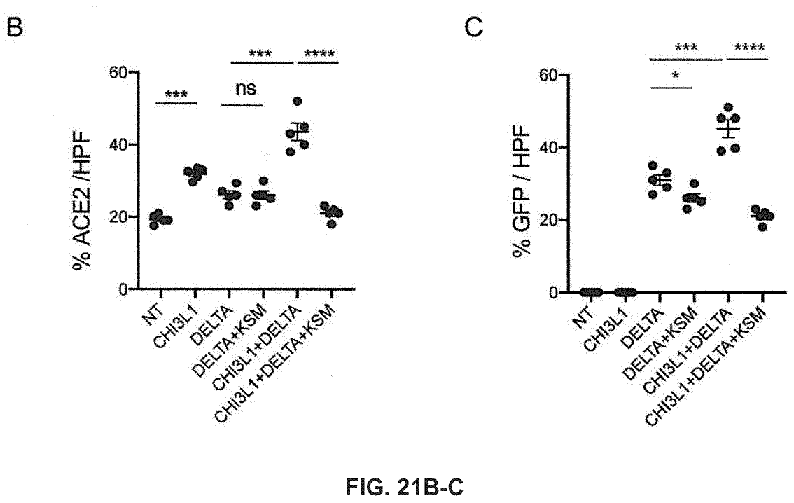

These findings were reinforced by immunocytochemical evaluations. These studies demonstrated that CHI3L1 augmented Calu-3 cell ACE2 accumulation and delta pseudovirus infection (FIG. 21). They also demonstrated that FRG abrogated the expression of delta pseudovirus infection at baseline and or after the administration of rCHI3L1 (FIG. 21). When viewed in combination, these studies demonstrate that kasugamycin targeting exogenous and or endogenous CHI3L1 effectively inhibits the uptake of pseudovirus with the α, β, γ, or δ S protein mutations.

### The Monoclonal Antibody “FRG” and Kasugamycin Inhibit Epithelial Uptake of Omicron Pseudotyped Viral Variants

Omicron variant has rapidly spread from its first appreciation as a highly mutated variant causing a localized outbreak in South Africa to the most common SC2 variant in the USA and the world (https://covid.cdc.gov/covid-data-tracker/#variant-proportions). Thus, studies were undertaken to determine if the therapies described above that target CHI3L1, ACE2 and SPP in ancestral α, β, γ, and δ pseudoviruses are also effective in pseudoviruses with omicron S protein mutations.

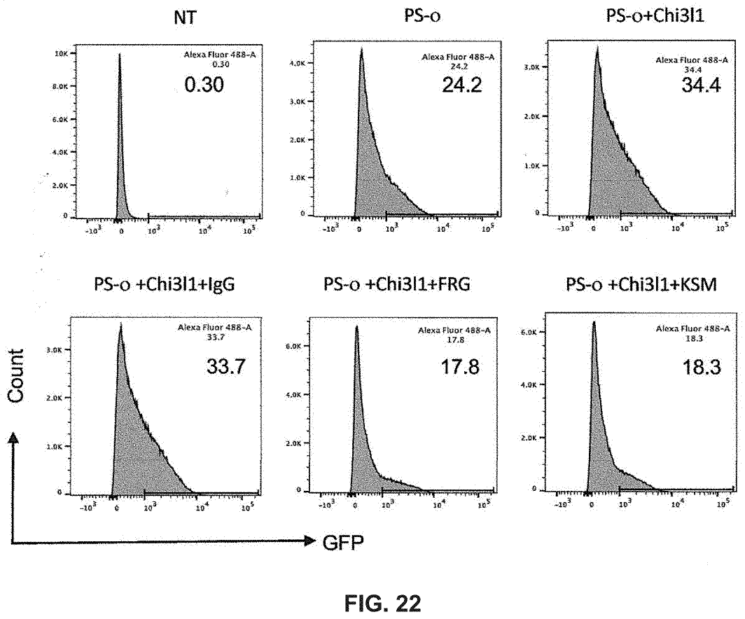

As was seen with the α, β, γ, and δ pseudoviruses, CHI3L1 was a potent stimulator of the uptake of pseudoviruses that contained omicron S proteins (FIG. 22). In addition, FRG and kasugamycin both inhibited epithelial cell uptake of pseudoviruses with omicron S protein mutations (FIG. 22). These studies demonstrate that antibodies or small molecule inhibitors that target CHI3L1 inhibit epithelial uptake of pseudoviruses with a wide range of S protein mutations including those seen in the omicron SC2 variant.

### Discussion

Coronaviruses are large enveloped single stranded viruses.¹¹⁷ Generally, the rates of nucleotide substitution of RNA viruses are fast and mainly the result of natural selection.¹¹⁸ This high error rate, and the subsequent rapidly evolving virus populations, can lead to the accumulation of amino acid mutations that affect the transmissibility, cell tropism, pathogenicity and or the responsiveness to vaccinations and or therapies.¹¹⁹ The SC2 VOC are known to manifest enhanced transmissibility and diminished vaccine effectiveness when compared to ancestral controls.¹²⁰ Their mutations are important causes of viral infection, the cause of new waves of illness and death and drivers of pandemic persistence.¹²¹ This can be readily appreciated in the rapid spread of the delta and omicron variants with the latter now accounting for 95.4% of SC2 infections in the USA (https://covid.cdc.gov/covid-data-tracker/#variant-proportions, as of Jan. 1, 2021). It can also be seen in delta's enhanced ability to replicate which drives the viral load up beyond what many other variants can do and outpaces the body's initial antiviral response.¹²² The fact that the spectrum of mutations in and characteristics of these variants differs from one another has complicated approaches to vaccination and therapy.

In light of the importance of the variants, especially delta and omicron, in COVID-19 studies were undertaken to determine if therapies could be developed by targeting host moieties that help to control many of the major VOC of SC2. In keeping with the importance of ACE2 and SPP in SC2 infection and the impressive ability of CHI3L1 to stimulate these moieties, these studies focused on the relationships between CHI3L1, ACE2 in infections caused by the α, β, γ, δ, and ∘ variants. The data presented above demonstrates that CHI3L1 stimulates the infection caused by these VOC by stimulating the expression and accumulation of ACE2 and SPP. The data also demonstrate that antibody-based and small molecule inhibitors of CHI3L1 inhibit the infection of human epithelial cells by these major SC2 VOC including delta and omicron. In combination, the data suggest that CHI3L1 is a potential therapeutic target that can be manipulated to prevent or alter the natural history of SC2 infection caused by the current and possible future viral variants that utilize ACE2 and SPP.

The S glycoprotein of SC2 is located on the outer surface of the virion and undergoes cleavage into S1 and S2 subunits. The S1 subunit is further divided into a receptor binding domain (RBD) and an N-terminal domain (NTD) which serve as potential targets for neutralization in response to antisera and or antibodies induced by vaccines.¹²³ Genetic variation in SC2 can have important implications for disease pathogenesis especially if the alterations involve the RBD. In keeping with this concept, the SC2 VOC have impressive mutations in the viral S proteins with alterations in RBD and NTD.¹²⁴ Three of the VOC have N501Y alterations which augment viral attachment to ACE2 and subsequent host cell infection.¹²⁵ Omicron has 37 amino acid mutations in its S protein.¹²⁶ Fifteen of these mutations are in the RBD and nine are in the RBM which is the subdomain of the RBD that directly interacts with ACE2.¹²⁷ When viewed in combination, these studies highlight the importance of the viral S proteins and host ACE2 and SPP in the responses induced by SC2 variants. Because our data demonstrates that CHI3L1 is a potent stimulator of ACE2 and SPP they also provide a mechanism by which CHI3L1-based interventions can be effective therapies in all SC2 variants that utilize ACE2 and SPP to mediate viral infection.

Antibodies against the SC2 spike proteins are an evolving and important part of the immune response to SC2 and treatment tool kit against COVID 19. Because the S protein of omicron is heavily mutated, the therapeutic efficacy of vaccine-induced antibodies and commercial monoclonal anti-spike protein antibodies have been characterized. These studies demonstrated that vaccine-induced antibodies can manifest diminished therapeutic efficacy compared to ancestral and other SC2 variants like delta.¹²⁸ In keeping with these findings, antibodies from Regeneron Inc and Eli Lilly Inc have been noted to manifest diminished potency against omicron while manifesting impressive efficacy against the delta and other variants.¹²⁹ Our studies demonstrate that the anti-CHI3L1 antibody FRG and kasugamycin, an inhibitor of CHI3L1 decrease the expression of ACE2 and the ability of the ancestral and the α, β, γ, δ, and ∘ variants to infect epithelial cells. This led us to hypothesize that FRG and kasugamycin could decrease the infection and spread of all SC2 variants that utilize ACE2 and SPP to elicit cell infection. In keeping with the instant findings, recent studies have demonstrated that omicron infection requires ACE2, and that omicron binds to ACE2 is more avidly than the binding of delta to ACE2.¹³⁰ This supports our contention that interventions that target CHI3L1 can be effective in the treatment of viruses that utilize ACE to infect epithelial cells.

Variants of interest (VOI) are defined as viral variants with specific genetic markers that may alter the transmissibility and or susceptibility of the virus to vaccination or therapeutic interventions when compared to ancestral strains.¹³¹ If the features of the variants are subsequently appreciated to exist, the variant is then reclassified as a VOC. As of Jun. 22, 2021, there were seven VOI including epsilon, zeta, eta, theta, kappa, and lambda. More recently, epsilon and Mu have been reclassified as a VOC.¹³² In all cases, ACE2 is presumed to be needed for infection by these viral variants. In keeping with this presumption, we believe that CHI3L1 will also regulate VOI infection, replication and symptom generation by altering ACE2 and SPP.

Studies from our laboratory and others have demonstrated that CHI3L1 is a critical regulator of inflammation and innate immunity and a stimulator of type 2 immune responses, fibroproliferative repair and angiogenesis.¹³³ These studies also demonstrated that CHI3L1 is increased in the circulation of patients that are older than 60 years of age and patients with a variety of comorbid diseases including obesity, cardiovascular disease, kidney disease, diabetes, chronic lung disease and cancer.¹³⁴ In keeping with these findings, we focused recent efforts on the development of CHI3L1-based interventions for these disorders. One of the most effective was a monoclonal antibody raised against amino acid 223-234 of CHI3L1 which is now called FRG. There are a number of reasons to believe that FRG can be an effective therapy in COVID 19. First, as noted by our laboratory¹³⁵ and in the studies noted above, it is a potent inhibitor of CHI3L1 stimulation of ACE2 and SPP which decreases the infection of epithelial cells by SC2. In addition, CHI3L1 is a potent stimulator of type 2 immune responses and type 2 and type 1 immune responses counter regulate each other. As a consequence, anti-CHI3L1 augments type 1 immune responses which have potent antiviral properties. Anti-CHI3L1 also inhibits the abnormal fibroproliferative repair responses that are seen in pathologic tissue fibrosis such as that seen in lungs from patients with COVID 19 who require prolonged mechanical ventilation. The present studies add to these insights by highlighting the ability of FRG to inhibit the infection of epithelial cells by the α, β, γ, δ, and ∘ SC2 VOC. When viewed in combination, these studies suggest that FRG is a potent therapeutic that can be used to prevent or diminish SC2 infection and or the COVID 19 disease manifestations induced by SC2 and its major variants while augmenting type 1 antiviral responses and controlling tissue fibrosis.

REGEN-COV-2 is a combination of the monoclonal antibodies casirivimab and imdevimab that bind to non-competing epitopes of the RBD of the S protein of SC2.¹³⁶ When administered via a subcutaneous route, iREGEN-COV2 markedly decreases the risk of hospitalization or death among high-risk persons with COVID 19. Subcutaneous REGEN-COV2 also prevents symptomatic infection in previously uninfected household contracts of infected persons and decreases the duration of the symptoms and the titers of the virus after SC2 infection.¹³⁷ Because the SC2 VOC have S protein mutations that involve the RBD, one can appreciate the importance of combining two antibodies that target different RBD epitopes to allow these antibodies to neutralize the various VOC including alpha, beta, gamma, delta and epsilon.¹³⁸ Because FRG and casirivimab/imdevimab control SC2 via different mechanisms, it is tempting to speculate that additive or synergistic antiviral and or anti-disease effects including preexposure and postexposure prophylaxis will be seen when FRG and REGEN-COV2 are administered simultaneously. One can also see how the administration of FRG and REGEN-COV2 in combination could protect against the selection of resistant SC2 variants.

Kasugamycin was discovered in 1965 in Streptomyces kasugaensis and has proven to have antibacterial and antifungal properties.¹³⁹ Since the 1960s, it has been employed as a pesticide to combat agricultural diseases like rice blast fungus and, as a result, has been extensively studied by the Environment Protection Agency (EPA).¹⁴⁰ Most recently Kasugamycin was shown to inhibit influenza and other viral infections.¹⁴¹ Previous studies from our laboratory have added to our understanding of kasugamycin by demonstrating that it is a powerful inhibitor of CHI3L1 induction of ACE2 and SPP that also inhibits type 2 adaptive immune responses and pathologic fibrosis.¹⁴² Importantly, the studies in this submission go further by demonstrating that these CHI3L1-based effects of kasugamycin can be seen in the ancestral and alpha, beta, gamma, delta and omicron SC2 VOC. When viewed in combination, these observations suggest that kasugamycin can be used as a prophylactic or therapeutic in COVID 19. This is an interesting concept because kasugamycin can be given via an intravenous or oral route and is known to have minimal toxicity in man.¹⁴³

At the onset of the SC2 pandemic, there was an urgency to mitigate this new viral illness. Since then, significant progress has been made in the treatment of COVID 19 due to intense research efforts that resulted in novel therapeutics and vaccine development at an unprecedented rate. The progress that was made, however, was diminished by the appearance of SC2 viral variants, particularly delta and omicron. It is now known that SC2 infection results in a disease with two phases. The early phase is characterized by cell infection and viral replication and the latter phase is characterized by a robust host antiviral immune response.¹⁴⁴ Current, therapies that are used in the early phase of SC2 infection include antivirals like remdesivir and anti-SC2 monoclonal antibody pairings like bamlanivimab/etesvimab and casirivimab/imdevfimab. When inflammation and a robust immune response have been triggered, anti-inflammatories like dexamethasone and immunomodulators are available.

The present studies add to our understanding of the therapies for the early phase of SC2 by demonstrating that the inhibition of ChI3L1 with FRG and or Kasugamycin ameliorates the infection induced by the α, β, γ, δ, and ∘ SC2 VOC. This raises the exciting possibility that FRG or Kasugamycin, alone or in combination with each other or other SC2 monoclonals, can have powerful prophylactic effects and or inhibit viral infection in SC2-exposed individuals. They also demonstrate that FRG and Kasugamycin can directly diminish viral replication and, by decreasing viral load, decrease disease pathology and severity.

### SUMMARY

COVID 19 is the disease caused by severe acute respiratory syndrome coronavirus-2 (SARS-CoV-2; SC2) which has caused a world-wide pandemic with striking morbidity and mortality. Evaluation of early SC2 strains suggested limited viral genetic diversity. However, genetic and epidemiologic investigations in the interim have revealed impressive genetic variability. Many of these viral variants are now defined as variants of concern (VOC) based on genetic alterations in their spike (S) and other proteins that cause enhanced transmissibility, decreased susceptibility to antibody neutralization or therapeutics and or their ability to induce severe disease. The delta (6) and omicron (o) variants are particularly problematic based on their impressive and unprecedented transmissibility and ability to cause break through infections. The delta variant also accumulates at high concentrations in host tissues and has caused waves of lethal disease. SC2 infection is mediated by S protein binding to cellular ACE2 receptors and subsequent S protein protease processing. Because studies from our laboratory have demonstrated that chitinase 3-like-1 (CHI3L1) stimulates ACE2 and S priming proteases, studies were undertaken to determine if interventions that target CHI3L1 are effective inhibitors of SC2 viral variant infection. The above-presented data demonstrate that CHI3L1 augments epithelial cell infection by pseudoviruses that express the alpha, beta, gamma, delta or omicron S proteins and that the CHI3L1 inhibitors anti-CHI3L1 and kasugamycin inhibit epithelial cell infection by these VOC pseudovirus moieties. Thus, CHI3L1 is a universal, VOC-independent therapeutic target in COVID 19.

### CONCLUSIONS

The studies described above indicate that: (1) chitinase 3 like 1 (CHI3L1) stimulates/upregulates ACE2 and a few of the spike activating proteases (SAPs) required for the spike protein to enter the host cells; and (2) this CHI3L1 stimulation of ACE2 and the SAPs was fully reversed by a CHI3L1 inhibitor. These data suggest administering a therapeutically effective amount of an inhibitor of CHI3L1 to subject at risk of, or afflicted with, an infection induced by a virus that enters host cells via the ACE2 cellular receptor can prevent, revert, or treat the viral infection.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain, using no more than routine experimentation, many equivalents to the specific embodiments described herein. Such equivalents are intended to be encompassed by the following claims.

### REFERENCES

¹ Perlman, S. and Netland, J. (2009). “Coronaviruses post-SARS: Update on replication and pathogenesis.” Nat. Rev. Microbiol. 7, 439-450.
² Hulswit, R. J. G., et al. (2016). “Coronavirus spike protein and tropism changes.” Adv Virus Res. 96: 29-57.
³ Li, F. (2013). “Receptor recognition and cross-species infections of SARS coronavirus.” Antiviral Res. 100: 246-254.
⁴ Li, F. (2016). “Structure, function, and evolution of coronavirus spike proteins.” Annu. Rev. Virol. 3: 237-261.
⁵ Graham, R. L., et al. (2013). “A decade after SARS: strategies for controlling emerging coronaviruses.” Nat. Rev. Microbiol. 11: 836-848.
⁶ Cui, J., et al. (2019). “Origin and evolution of pathogenic coronaviruses.” Nat. Rev. Microbiol. 17: 181-192.
⁷ Li, Q., et al. (2020). “Early transmission dynamics in Wuhan, China, of novel coronavirus-infected pneumonia.” N. Engl. J. Med. 382: 1199-1207.
⁸ Du, L., et al. (2009). “The spike protein of SARS-CoV-a target for vaccine and therapeutic development.” Nat. Rev. Microbiol. 7: 226-236.
⁹ Du, L., et al. (2017). “MERS-CoV spike protein: a key target for antivirals.” Expert Opin. Ther. Targets 21: 131-143.
¹⁰ Liu, S., et al. (2004). “Interaction between heptad repeat 1 and 2 regions in spike protein of SARS-associated coronavirus: implications for virus fusogenic mechanism and identification of fusion inhibitors.” Lancet 363: 938-947.
¹¹ Li, F., et al. (2005). “Structure of SARS coronavirus spike receptor-binding domain complexed with receptor.” Science 309: 1864-1868.
¹² Li, W, et al. (2003). “Angiotensin-converting enzyme 2 is a functional receptor for the SARS coronavirus.” Nature 426: 450-454.
¹³ Raj, V. S., et al. (2003). “Dipeptidyl peptidase 4 is a functional receptor for the emerging human coronavirus-EMC.” Nature 495: 251-254.
¹⁴ Zhou, P., et al. (2020). “A pneumonia outbreak associated with a new coronavirus of probable bat origin.” Nature 579: 270-273.
¹⁵ Harmsen, M. M. and De Haard, H. J. (2007). “Properties, production, and applications of camelid single-domain antibody fragments.” Applied Microbiology and Biotechnology. 77 (1): 13-22.
¹⁶ Padlan, E. A., et al. (1995). “Identification of specificity-determining residues in antibodies.” FASEB J. 9:133-139.
¹⁷ MacCallum, R. M., et al. (1996). “Antibody-antigen interactions: contact analysis and binding site topography.” J. Mol. Biol. 262(5):732-745.
¹⁸ Chothia, C. et al. (1987). “Canonical structures for the hypervariable regions of immunoglobulins.” J. Mol. Biol. 196: 901-917.
¹⁹ Chothia, C. et al. (1989). “Conformations of immunoglobulin hypervariable regions.” Nature 342: 877-883 (1989).
²⁰ U.S. Pat. No. 10,253,111; Elias, J. A. et al., “Methods and compositions relating to anti-CHI3L1 antibody reagents.” Issued: Apr. 9, 2019.
²¹ Kabat, E. A., et al. (1991). SEQUENCES OF PROTEINS OF IMMUNOLOGICAL INTEREST. Fifth Edition, U.S. Department of Health and Human Services, NIH Publication No. 91 3242.
²² U.S. Pat. No. 5,585,089; Queen, C. L. et al., “Humanized immunoglobulins.” Issued: Dec. 17, 1996.
²³ U.S. Pat. No. 6,824,989. Eisinger, D. et al., “Recombinant monoclonal antibody to phosphotyrosine-containing proteins.” Issued: Nov. 30, 2004.
²⁴ U.S. Pat. No. 6,835,823; Le, J. et al. “Anti-TNF antibodies and peptides of human tumor necrosis factor.” Issued: Dec. 28, 2004.
²⁵ THE MERCK MANUAL OF DIAGNOSIS AND THERAPY, 19^{th} Edition, (2011). Published by Merck Sharp & Dohme Corp., (ISBN 978-0-911910-19-3).
²⁶ THE ENCYCLOPEDIA OF MOLECULAR CELL BIOLOGY AND MOLECULAR MEDICINE, (1999-2012) Porter, R. S., et al. (eds.), published by Blackwell Science Ltd., (ISBN 9783527600908).
²⁷ MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
²⁸ IMMUNOLOGY, (2006). Luttmann, W. et al. (eds.), published by Elsevier (ISBN: 9780120885442).
²⁹ JANEWAY'S IMMUNOBIOLOGY, (2014). Murphy, K., et al. (eds.), published by Taylor & Francis Limited, (ISBN 0815345305, 9780815345305).
³⁰ LEWIN'S GENES XI, 11^{th} edition, (2014). Krebs, J. E., et al. (eds.), published by Jones & Bartlett Publishers (ISBN-1449659055).
³¹ MOLECULAR CLONING: A LABORATORY MANUAL, 4^{th} edition, (2012). Green M. R. and Sambrook, J. (eds.), published by Cold Spring Harbor Laboratory Press, Cold Spring Harbor, N.Y., USA (ISBN 1936113414).
³² BASIC METHODS IN MOLECULAR BIOLOGY, (2012). Davis et al. (eds.), published by Elsevier Science Publishing, Inc., New York, USA (ISBN 044460149X).
³³ LABORATORY METHODS IN ENZYMOLOGY: DNA, 1^{st} edition, (2013). Lorsch, J. (ed.), published by Elsevier (ISBN 0124199542).
³⁴ CURRENT PROTOCOLS IN MOLECULAR BIOLOGY (CPMB), (2014). Ausubel, F. M. (ed.), published by John Wiley and Sons (ISBN 047150338X, 9780471503385).
³⁵ CURRENT PROTOCOLS IN PROTEIN SCIENCE (CPPS), (2005). Coligan, J. E. (ed.), published by John Wiley and Sons, Inc.
³⁶ CURRENT PROTOCOLS IN IMMUNOLOGY (CPI), (2003). Coligan, J. E., et al. (eds.), published by John Wiley and Sons, Inc. (ISBN 0471142735, 9780471142737).
³⁷ Zhou, Y., et al. (2014). “Chitinase 3-like 1 Suppresses Injury and Promotes Fibroproliferative Responses in Mammalian Lung Fibrosis.” Sci. Transl. Med. 6(240): 240ra76.
³⁸ Hou, Y. J., et al. (2020). “SARS-CoV-2 D614G variant exhibits efficient replication ex vivo and transmission in vivo.” Science 370(6523): 1464-1468.
³⁹ Plante, J. A., et al. (2021). “Spike mutation D614G alters SARS-CoV-2 fitness.” Nature 592: 116-121.
⁴⁰ Wise, J. (2021). “Covid-19: The E484K mutation and the risks it poses.” BMJ2021; 372: n359.
⁴¹ Wise J. (2020). “Covid-19: New coronavirus variant is identified in U K.” BMJ2020; 371: m4857.
⁴² Peacock, Sharon (22 Dec. 2020). “Here's what we know about the new variant of coronavirus”. The Guardian.
⁴³ “Emerging SARS-CoV-2 Variants”. cdc.org (Science brief). Centers for Disease Control and Prevention. Jan. 28, 2021.
⁴⁴ “South Africa announces a new coronavirus variant.” The New York Times. Dec. 18, 2020.
⁴⁵ Mkhize, Z. (2020). “Update on Covid-19.” Dec. 18, 2020 Press release. South Africa. COVID-19 South African Online Portal.
⁴⁶ Faria, N. R., et al. (12 Jan. 2021). “Genomic characterisation of an emergent SARS-CoV-2 lineage in Manaus: preliminary findings.” Virological.
⁴⁷ “Japan finds new coronavirus variant in travelers from Brazil.” Japan Today, Jan. 11, 2021.
⁴⁸ Faria, N. R., et al. (12 Jan. 2021). “Genomic characterisation of an emergent SARS-CoV-2 lineage in Manaus: preliminary findings.” Virological.
⁴⁹ Id.
⁵⁰ International Patent Publication WO 2019/060675; Chupp, G. and Cohn, L., “Anti-YKL40 antibodies and methods of use.” Published: Mar. 28, 2019.
⁵¹ BIOCHEMISTRY, 2^{nd} edition, (1975). Lehninger, A. L. (ed.), published by Worth Publishers, New York at pp. 73-75.
⁵² Fujii, I. (2004). “Antibody affinity maturation by random mutagenesis.” Methods Mol. Biol. 248: 345-349.
⁵³ Maus, M. V., et al. (2014). “Antibody-modified T cells: CARs take the front seat for hematologic malignancies.” Blood 123: 2624-2635.
⁵⁴ Reardon, D. A., et al. (2014). “Immunotherapy advances forglioblastoma.” Neuro-Oncology 16: 1441-1458.
⁵⁵ Hoyos, V., et al. (2012). “Genetic modification of human T lymphocytes for the treatment of hematologic malignancies.” Haematologica 97:1622-1631.
⁵⁶ Byrd, J. C., et al. (2014). “Entering the era of targeted therapy for chronic lymphocytic leukemia: impact on the practicing clinician.” J. Clin. Oncol. 32: 3039-3047.
⁵⁷ Maher, J. and Wilkie, S. (2009). “CAR Mechanics: Driving T Cells into the MUC of Cancer.” Cancer Res. 69: 4559-4562.
⁵⁸ Tamada, K., et al. (2012). “Redirecting Gene-Modified T Cells toward Various Cancer Types Using Tagged Antibodies.” Clin. Cancer Res. 18: 6436-6445.
⁵⁹ Rockwood, D. N., et al. (2011). “Materials fabrication from Bombyx mori silk fibroin.” Nature Protocols 6: 1612-1631.
⁶⁰ U.S. Published Application No. 2011/0167602; Altman, G. H. et al., “Immunoneutral silk fiber-based medical devices.” Published: Jul. 14, 2011.
⁶¹ U.S. Published Application No. 2011/0009960; Altman, G. H. et al., “Prosthetic fabric structure.” Published: Jan. 13, 2011.
⁶² U.S. Published Application No. 2012/0296352; Altman, G. H. et al., “Sericin extracted fabrics.” Published: Nov. 22, 2012.
⁶³ U.S. Pat. No. 8,172,901; Altman, G. H. et al., “Prosthetic device and method of manufacturing the same.” Issued: May 8, 2012.
⁶⁴ Malumbres M., et al., (2008). “CDK inhibitors in cancer therapy: What is next?” Trends in Pharmacological Sciences 29 (1): 16-21.
⁶⁵ Source: AdooQ Bioscience https://www.adooq.com/
⁶⁶ Harmon, A. D., et al. (1979). “The structure of rohitukine, the main alkaloid of Amoora rohituka (syn. Aphanamixis polystachya) (Meliaceae).” Tetrahedron Lett. 20 (1): 721-724.
⁶⁷ Lakdawala, A. D., et al. (1988). “Immunopharmacological potential of rohitukine: a novel compound isolated from the plant Dysoxylum binectariferum.” Asia Pac. J. Pharmcol. 3 (1): 91-98.
⁶⁸ U.S. Pat. No. 5,317,095; Suzuki, Y. et al. “Alpha-D-glycosyl kasugamycin, its preparation, and antibacterial agent containing the same.” Issued: May 31, 1994. U.S. Pat. No. 4,554,269; Takaya, T. et al. “Kasugamycin derivatives, pharmaceutical compositions and method of use.” Issued: Nov. 19, 1985. U.S. Pat. No. 3,968,100; Umezawa, H. and Suhara, Y. “Guanidio derivatives of kasugamycin and their production.” Issued: Jul. 6, 1976.
⁶⁹ REMINGTON'S PHARMACEUTICAL SCIENCES, 18^{th} edition, (1990). A. R. Gennaro (ed.), published by MackPubl., Easton, PA
⁷⁰ International Patent Publication WO 2019/036666; Elias, J. A. et al., “Methods and Compositions Relating to the Treatment of Fibrosis.” Published: Feb. 21, 2019.
⁷¹ McNamara, J. O., et al. (2006). “Cell type-specific delivery of siRNAs with aptamer-siRNA chimeras.” Nat. Biotechnol. 24:1005-1015.
⁷² Kim, S. H., et al. (2008). “Local and systemic delivery of VEGF siRNA using polyelectrolyte complex micelles for effective treatment of cancer.” Journal of Controlled Release 129(2):107-116.
⁷³ U.S. Pat. No. 7,427,605; Davis, M. E. et al., “Inhibitors of ribonucleotide reductase subunit 2 and uses thereof.” Issued: Sep. 23, 2008.
⁷⁴ Ikeda, Y. and Taira, K. (2006). “Ligand-targeted delivery of therapeutic siRNA.” Pharmaceutical Res. 23:1631-1640.
⁷⁵ Soutschek, J., et al. (2004). “Therapeutic silencing of an endogenous gene by systemic administration of modified siRNAs.” Nature 432: 173-178.
⁷⁶ Lorenze, C., et al. (2004). “Steroid and lipid conjugates of siRNAs to enhance cellular uptake and gene silencing in liver cells.” Bioorg. Med. Chem. Lett. 14: 4975-4977.
⁷⁷ Hoffmann, M., et al. (March 2020). “SARS-CoV-2 Cell Entry Depends on ACE2 and TMPRSS2 and Is Blocked by a Clinically Proven Protease Inhibitor.” Cell 181(2): 271-280.
⁷⁸ Rahman, N., et al. (May 2020). “Virtual screening of natural products against type II transmembrane serine protease (TMPRSS2), the priming agent of Coronavirus 2 (SARS-CoV-2).” Molecules 25(10): 2271.
⁷⁹ Lee, C. G., et al. (2009). “Role of breast regression protein 39 (BRP-39)/chitinase 3-like-1 in Th2 and IL-13-induced tissue responses and apoptosis.” J. Exp. Med. 206(5):1149-66.
⁸⁰ Sohn, M. H., et al. (2010). “The chitinase-like proteins breast regression protein-39 and YKL-40 regulate hyperoxia-induced acute lung injury.” Am. J. Resp. and Critical Care Med. 182(7): 918-28.
⁸¹ Foster, K. A., et al. (1998). “Characterization of the A549 cell line as a type II pulmonary epithelial cell model for drug metabolism.” Experimental Cell Research. 243(2): 359-366.
⁸² Product description for ATCC@ PCS-301-010™—Primary Airway Epithelial Cells; Normal, Human (HSAEC).
⁸³ Zhu, Y., et al. (2010). “Cultured Human Airway Epithelial Cells (Calu-3): A Model of Human Respiratory Function, Structure, and Inflammatory Responses.” Critical Care Research and Practice. 2010: 1-8.
⁸⁴ See, e.g., Hoffmann, M., et al. (March 2020). “SARS-CoV-2 Cell Entry Depends on ACE2 and TMPRSS2 and Is Blocked by a Clinically Proven Protease Inhibitor.” Cell 181(2): 271-280; Matsuyama, S., et al. (March 2020). “Enhanced isolation of SARS-CoV-2 by TMPRSS2-expressing cells.” PNAS 117 (13) 7001-7003.
⁸⁵ Coutard, B., et al. (2020). “The spike glycoprotein of the new coronavirus 2019-nCoV contains a furin-like cleavage site absent in CoV of the same clade.” Antiviral Res. 176: 104742.
⁸⁶ Lee, C. G., et al. (2009). “Role of breast regression protein 39 (BRP-39)/chitinase 3-like-1 in Th2 and IL-13-induced tissue responses and apoptosis.” J. Exp. Med. 206: 1149-1166.
⁸⁷ Lee, C. G., et al. (2004). “Early growth response gene 1-mediated apoptosis is essential for trans-forming growth factor, β₁-induced pulmonary fibrosis.” J. Exp. Med. 200: 377-389.
⁸⁸ Islam, M. R., et al. (2020). “Genome-wide analysis of SARS-CoV-2 virus strains circulating worldwide implicates heterogeneity.” Sci. Rep. 10(1): 14004.
⁸⁹ Id.
⁹⁰ Korber, B., et al. (2020). “Tracking Changes in SARS-CoV-2 Spike: Evidence that D614G Increases Infectivity of the COVID-19 Virus.” Cell 182(4): 812-827.
⁹¹ van Dorp, L., et al. (2020). “Emergence of genomic diversity and recurrent mutations in SARS-CoV-2.” Infection, Genetics and Evolution 83: 104351.
⁹² Benvenuto, D., et al. (2020). “Evolutionary analysis of SARS-CoV-2: how mutation of Non-Structural Protein 6 (NSP6) could affect viral autophagy.” J. Infect. 81(1): e24-e27.
⁹³ Pachetti, M., et al. (2020). “Emerging SARS-CoV-2 mutation hot spots include a novel RNA-dependent-RNA polymerase variant.” J. Transl. Med. 18(1): 179.
⁹⁴ Comandatore, F., et al. (2020). “Identification of variable sites in Sars-CoV-2 and their abundance profiles in time.” bioRxiv. 2020.04.30.071027.
⁹⁵ Fisman, D. N. and Tuite, A. R. (2021). “Progressive Increase in Virulence of Novel SARS-CoV-2 Variants in Ontario, Canada.” medRxiv preprint. doi.org/10.1101/2021.07.05.21260050
⁹⁶ Sheikh, A., et al. (2021). “SARS-CoV-2 Delta VOC in Scotland: demographics, risk of hospital admission, and vaccine effectiveness.” The Lancet 397(10293): 2461-2462.
⁹⁷ Stowe, J., et al. (2021). “Effectiveness of COVID-19 vaccines against hospital admission with the Delta (B.1.617.2) variant.” UK Health Security Agency. https://khub.net/web/phe-national/public-library/-/document_library/v2WsRK3ZIEig/view_file/479607329
⁹⁸ Thompson, M. G., et al. (2021). “Prevention and Attenuation of COVID-19 with the BNT162b2 and mRNA-1273 Vaccines.” N. Engl. J. Med. July 22; 385(4): 320-329.
⁹⁹ Chen N, Zhou M, Dong X, Qu J, Gong F, Han Y, et al. “Epidemiological and clinical characteristics of 99 cases of 2019 novel coronavirus pneumonia in Wuhan, China: a descriptive study.” Lancet. 2020; 395(10223): 507-513;
Huang C, Wang Y, Li X, Ren L, Zhao J, Hu Y, et al. “Clinical features of patients infected with 2019 novel coronavirus in Wuhan, China. Lancet. 2020; 395(10223): 497-506;
Li Q, Guan X, Wu P, Wang X, Zhou L, Tong Y, et al. “Early Transmission Dynamics in Wuhan, China, of Novel Coronavirus-Infected Pneumonia.” N Engl J Med. 2020; 382(13): 1199-207;
Wang D, Hu B, Hu C, Zhu F, Liu X, Zhang J, et al. “Clinical Characteristics of 138 Hospitalized Patients With 2019 Novel Coronavirus-Infected Pneumonia in Wuhan, China.” JAMA. 2020;
Guan W J, Ni Z Y, Hu Y, Liang W H, Ou C Q, He J X, et al. “Clinical Characteristics of Coronavirus Disease 2019 in China.” N Engl J Med. 2020; 382(18): 1708-1720;
Wu Z, and McGoogan J M. “Characteristics of and Important Lessons From the Coronavirus Disease 2019 (COVID-19) Outbreak in China: Summary of a Report of 72314 Cases From the Chinese Center for Disease Control and Prevention.” JAMA. 2020;
CSSE J H U. “COVID-19 Dashboard by the Center for Systems Science and Engineering (CSSE) at Johns Hopkins University.” https://coronavirusihuedu/maphtml 2021;
Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹⁰⁰ Lake M A. “What we know so far: COVID-19 current clinical knowledge and research.” Clin Med (Lond). 2020; 20(2): 124-7.
¹⁰¹ Inciardi R M, Solomon S D, Ridker P M, and Metra M. “Coronavirus 2019 Disease (COVID-19), Systemic Inflammation, and Cardiovascular Disease.” J Am Heart Assoc. 2020; 9(16): e017756; Gupta A, Madhavan M V, Sehgal K, Nair N, Mahajan S, Sehrawat T S, et al. “Extrapulmonary manifestations of COVID-19.” Nat Med. 2020; 26(7): 1017-32.
¹⁰² Millet J K, and Whittaker G R. “Host cell proteases: Critical determinants of coronavirus tropism and pathogenesis.” Virus Res. 2015; 202: 120-34;
Coutard B, Valle C, de Lamballerie X, Canard B, Seidah N G, and Decroly E. “The spike glycoprotein of the new coronavirus 2019-nCoV contains a furin-like cleavage site absent in CoV of the same clade.” Antiviral Res. 2020; 176: 104742;
Hoffmann M, Kleine-Weber H, Schroeder S, Kruger N, Herrler T, Erichsen S, et al. “SARS-CoV-2 Cell Entry Depends on ACE2 and TMPRSS2 and Is Blocked by a Clinically Proven Protease Inhibitor.” Cell. 2020; 181(2): 271-280 e8;
Letko M, Marzi A, and Munster V. “Functional assessment of cell entry and receptor usage for SARS-CoV-2 and other lineage B betacoronaviruses.” Nat Microbiol. 2020; 5(4): 562-569.
¹⁰³ Hoffmann M, Kleine-Weber H, Schroeder S, Kruger N, Herrler T, Erichsen S, et al. “SARS-CoV-2 Cell Entry Depends on ACE2 and TMPRSS2 and Is Blocked by a Clinically Proven Protease Inhibitor.” Cell. 2020; 181(2): 271-280 e8;
Bourgonje A R, Abdulle A E, Timens W, Hillebrands J L, Navis G J, Gordijn S J, et al. “Angiotensin-converting enzyme 2 (ACE2), SARS-CoV-2 and the pathophysiology of coronavirus disease 2019 (COVID-19).” J Pathol. 2020;
Zhang H, Rostami M R, Leopold P L, Mezey J G, O'Beirne S L, Strulovici-Barel Y, et al. “Expression of the SARS-CoV-2 ACE2 Receptor in the Human Airway Epithelium.” Am J Respir Crit Care Med. 2020.
¹⁰⁴ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹⁰⁵ Baric R S. “Emergence of a Highly Fit SARS-CoV-2 Variant.” New England Journal of Medicine. 2020.
¹⁰⁶ Id.
Korber B, Fischer W M, Gnanakaran S, Yoon H, Theiler J, Abfalterer W, et al. “Tracking Changes in SARS-CoV-2 Spike: Evidence that D614G Increases Infectivity of the COVID-19 Virus.” Cell. 2020; 182(4):812-27.e19.
¹⁰⁷ Baric R S. “Emergence of a Highly Fit SARS-CoV-2 Variant.” New England Journal of Medicine. 2020; Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹⁰⁸ Id.
¹⁰⁹ del Rio C, Malani P N, and Omer S B. “Confronting the Delta Variant of SARS-CoV-2, Summer 2021.” JAMA. 2021; 326(11):1001-1002;
Dhar M S, Marwal R, Radhakrishnan V S, Ponnusamy K, Jolly B, Bhoyar R C, et al. “Genomic characterization and Epidemiology of an emerging SARS-CoV-2 variant in Delhi, India.” medRxiv. 2021:2021.06.02.21258076.
¹¹⁰ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹¹¹ Shastri J, Parikh S, Aggarwal V, Agrawal S, Chatterjee N, Shah R, et al. “Severe SARS-CoV-2 Breakthrough Reinfection With Delta Variant After Recovery From Breakthrough Infection by Alpha Variant in a Fully Vaccinated Health Worker.” Frontiers in Medicine. 2021; 8(1379).
¹¹² Cameroni E, Saliba C, Bowen J E, Rosen L E, Culap K, Pinto D, et al. “Broadly neutralizing antibodies overcome SARS-CoV-2 Omicron antigenic shift.” bioRxiv. 2021: 2021.12.12.472269.
¹¹³ Id;
Sheikh A, McMenamin J, Taylor B, and Robertson C. “SARS-CoV-2 Delta VOC in Scotland: demographics, risk of hospital admission, and vaccine effectiveness.” Lancet. 2021; 397(10293): 2461-2462;
Greaney A J, Loes A N, Crawford K H D, Starr T N, Malone K D, Chu H Y, et al. “Comprehensive mapping of mutations in the SARS-CoV-2 receptor-binding domain that affect recognition by polyclonal human plasma antibodies.” Cell Host Microbe. 2021; 29(3):463-76.e6.
¹¹⁴ Lee C G, Cho S J, Kang M J, Chapoval S P, Lee P J, Noble P W, et al. “Early growth response gene 1-mediated apoptosis is essential for transforming growth factor beta1-induced pulmonary fibrosis.” The Journal of experimental medicine.” 2004; 200(3): 377-389.
¹¹⁵ Kamle S, Ma B, He C H, Akosman B, Zhou Y, Lee C M, et al. “Chitinase 3-like-1 is a Therapeutic Target That Mediates the Effects of Aging in COVID-19.” bioRxiv. 2021:2021.01.05.425478.
¹¹⁶ Lee J-H, Lee C-M, Kim M-O, Park J W, Kamle S, Akosman B, et al. “Kasugamycin is a novel chitinase 1 inhibitor with strong antifibrotic effects on pulmonary fibrosis.” bioRxiv. 2021:2021.02.25.432796;
Qi H, Jiang X, Ding Y, Liu T, and Yang Q. “Discovery of Kasugamycin as a Potent Inhibitor of Glycoside Hydrolase Family 18 Chitinases.” Frontiers in Molecular Biosciences. 2021; 8(111)
¹¹⁷ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹¹⁸ Giovanetti M, Benedetti F, Campisi G, Ciccozzi A, Fabris S, Ceccarelli G, et al. “Evolution patterns of SARS-CoV-2: Snapshot on its genome variants.” Biochem Biophys Res Commun. 2021; 538: 88-91.
¹¹⁹ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021;
Giovanetti M, Benedetti F, Campisi G, Ciccozzi A, Fabris S, Ceccarelli G, et al. “Evolution patterns of SARS-CoV-2: Snapshot on its genome variants.” Biochem Biophys Res Commun. 2021; 538: 88-91.
¹²⁰ Dubey A, Choudhary S, Kumar P, and Tomar S. “Emerging SARS-CoV-2 Variants: Genetic Variability and Clinical Implications.” Current Microbiology. 2021; 79(1): 20;
Chia P Y, Xiang Ong S W, Chiew C J, Ang L W, Chavatte J-M, Mak T-M, et al. “Virological and serological kinetics of SARS-CoV-2 Delta variant vaccine-breakthrough infections: a multi-center cohort study.” medRxiv. 2021:2021.07.28.21261295.
¹²¹ Dubey A, Choudhary S, Kumar P, and Tomar S. “Emerging SARS-CoV-2 Variants: Genetic Variability and Clinical Implications.” Current Microbiology. 2021; 79(1): 20.
¹²² Mlcochova P, Kemp S A, Dhar M S, Papa G, Meng B, Ferreira I A T M, et al. “SARS-CoV-2 B.1.617.2 Delta variant replication and immune evasion.” Nature. 2021; 599(7883): 114-119.
¹²³ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021;
Song W, Gui M, Wang X, and Xiang Y. “Cryo-EM structure of the SARS coronavirus spike glycoprotein in complex with its host cell receptor ACE2.” PLoS Pathog. 2018; 14(8): e1007236.
¹²⁴ Ou J, Zhou Z, Dai R, Zhang J, Zhao S, Wu X, et al. “V367F Mutation in SARS-CoV-2 Spike RBD Emerging during the Early Transmission Phase Enhances Viral Infectivity through Increased Human ACE2 Receptor Binding Affinity.” J Virol. 2021; 95(16): e0061721;
Shen L, Triche T J, Bard J D, Biegel J A, Judkins A R, and Gai X. “Spike Protein NTD mutation G142D in SARS-CoV-2 Delta VOC lineages is associated with frequent back mutations, increased viral loads, and immune evasion.” medRxiv. 2021: 2021.09.12.21263475.
¹²⁵ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹²⁶ Cameroni E, Saliba C, Bowen J E, Rosen L E, Culap K, Pinto D, et al. “Broadly neutralizing antibodies overcome SARS-CoV-2 Omicron antigenic shift.” bioRxiv. 2021: 2021.12.12.472269.
¹²⁷ Id.
¹²⁸ Liu L, Iketani S, Guo Y, Chan J F W, Wang M, Liu L, et al. “Striking Antibody Evasion Manifested by the Omicron Variant of SARS-CoV-2.” bioRxiv. 2021: 2021.12.14.472719;
VanBlargan L A, Errico J M, Halfmann P J, Zost S J, Crowe J E, Purcell L A, et al. “An infectious SARS-CoV-2 B.1.1.529 Omicron virus escapes neutralization by several therapeutic monoclonal antibodies.” bioRxiv. 2021: 2021.12.15.472828.
¹²⁹ Id.
¹³⁰ Golcuk M, Yildiz A, and Gur M. “The Omicron Variant Increases the Interactions of SARS-CoV-2 Spike Glycoprotein with ACE2.” bioRxiv. 2021:2021.12.06.471377.
¹³¹ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.
¹³² Id.
¹³³ Lee C G, Herzog E L, Ahangari F, Zhou Y, Gulati M, Lee C M, et al. “Chitinase 1 is a biomarker for and therapeutic target in scleroderma-associated interstitial lung disease that augments TGF-beta1 signaling.” J Immunol. 2012; 189(5): 2635-44;
Zhou Y, He C H, Yang D S, Nguyen T, Cao Y, Kamle S, et al. “Galectin-3 Interacts with the CH/3L1 Axis and Contributes to Hermansky-Pudlak Syndrome Lung Disease.” J Immunol. 2018; 200(6): 2140-2153;
Dela Cruz C S, Liu W, He C H, Jacoby A, Gornitsky A, Ma B, et al. “Chitinase 3-like-1 (Chi3/1) Regulation of Streptococcus pneumoniae Lung Infection.” Cell Host and Microbe. 2012; 12(1):34-46;
Zhou Y, Peng H, Sun H, Peng X, Tang C, Gan Y, et al. “Chitinase 3-like 1 suppresses injury and promotes fibroproliferative responses in Mammalian lung fibrosis.” Science translational medicine. 2014; 6(240): 240ra76;
Kang M J, Lee C G, Lee J Y, Dela Cruz C S, Chen Z J, Enelow R, et al. “Cigarette smoke selectively enhances viral PAMP-and virus-induced pulmonary innate immune and remodeling responses in mice. J Clin Invest. 2008; 118(8):2771-2784;
Kang M J, Yoon C M, Kim B H, Lee C M, Zhou Y, Sauler M, et al. “Suppression of NLRX1 in chronic obstructive pulmonary disease.” J Clin Invest. 2015; 125(6):2458-2462;
He C H, Lee C G, Dela Cruz C S, Lee C M, Zhou Y, Ahangari F, et al. “Chitinase 3-like 1 regulates cellular and tissue responses via IL-13 receptor alpha2.” Cell reports. 2013; 4(4): 830-841;
Lee C G, Hartl D, Lee G R, Koller B, Matsuura H, Da Silva C A, et al. “Role of breast regression protein 39 (BRP-39)/chitinase 3-like-1 in Th2 and IL-13-induced tissue responses and apoptosis.” The Journal of experimental medicine. 2009; 206(5):1149-66.
¹³⁴ Dela Cruz C S, Liu W, He C H, Jacoby A, Gornitsky A, Ma B, et al. “Chitinase 3-like-1 (Chi3/1) Regulation of Streptococcus pneumoniae Lung Infection.” Cell Host and Microbe. 2012; 12(1):34-46;
Garnero P, Mazieres B, Gueguen A, Abbal M, Berdah L, Lequesne M, et al. “Cross-sectional association of 10 molecular markers of bone, cartilage, and synovium with disease activity and radiological joint damage in patients with hip osteoarthritis: the ECHODIAH cohort.” J Rheumatol. 2005; 32(4):697-703;
Hakala B E, White C, and Recklies A D. “Human cartilage gp-39, a major secretory product of articular chondrocytes and synovial cells, is a mammalian member of a chitinase protein family.” J Biol Chem. 1993; 268(34):25803-25810;
Johansen J S, Christoffersen P, Moller S, Price P A, and Bendtsen F. “Serum YLK-40 is increased in patients with hepatic fibrosis.” J Hepatology. 2000; 32: 911-920;
Johansen J S, Jensen H S, and Price P A. “A new biochemical marker for joint injury. Analysis of YKL-40 in serum and synovial fluid.” Br J Rheumatol. 1993; 32(11): 949-55;
Kucur M, Isman F K, Karadag B, Vural V A, and Tavsanoglu S. “Serum YKL-40 levels in patients with coronary artery disease.” Coron Artery Dis. 2007; 18: 391-396;
Lee C G. “Chitin, chitinases and chitinase-like proteins in allergic inflammation and tissue remodeling.” Yonsei Med J. 2009; 50(1): 22-30;
Lee C G, Da Silva C A, Dela Cruz C S, Ahangari F, Ma B, Kang M J, et al. “Role of chitin and chitinase/chitinase-like proteins in inflammation, tissue remodeling and injury.” Annu Rev Physiol. 2011; 73: 479-501;
Lee C G, and Elias J A. “Role of breast regression protein-39/YKL-40 in asthma and allergic responses.” Allergy Asthma Immunol Res. 2010; 2(1): 20-27;
Matsuura H, Hartl D, Kang M J, Dela Cruz C S, Koller B, Chupp G L, et al. “Role of breast regression protein-39 in the pathogenesis of cigarette smoke-induced inflammation and emphysema.” Am J Respir Cell Mol Biol. 2011; 44(6): 777-786;
Nordenbaek C, Johansen J S, Junker P, Borregaard N, Sorensen O, and Price P A. “YKL-40, a matrix protein of specific granules in neutrophils, is elevated in serum of patients with community-acquired pneumonia requiring hospitalization.” J Infect Dis. 1999; 180(5): 1722-1726;
Ostergaard C, Johansen J S, Benfield T, Price P A, and Lundgren J D. “YKL-40 is elevated in cerebrospinal fluid from patients with purulent meningitis.” Clin Diagn Lab Immunol. 2002; 9(3): 598-604.
¹³⁵ Kamle S, Ma B, He C H, Akosman B, Zhou Y, Lee C M, et al. “Chitinase 3-like-1 is a Therapeutic Target That Mediates the Effects of Aging in COVID-19.” bioRxiv. 2021:2021.01.05.425478.
¹³⁶ O'Brien M P, Forleo-Neto E, Musser B J, Isa F, Chan K-C, Sarkar N, et al. “Subcutaneous REGEN-COVAntibody Combination to Prevent Covid-19.” New England Journal of Medicine. 2021; 385(13): 1184-1195.
¹³⁷ Id.
¹³⁸ Baum A, Fulton B O, Wloga E, Copin R, Pascal K E, Russo V, et al. “Antibody cocktail to SARS-CoV-2 spike protein prevents rapid mutational escape seen with individual antibodies.” Science. 2020; 369(6506):1014-1018;
Copin R, Baum A, Wloga E, Pascal K E, Giordano S, Fulton B O, et al. “The monoclonal antibody combination REGEN-COV protects against SARS-CoV-2 mutational escape in preclinical and human studies.” Cell. 2021; 184(15): 3949-61.e11;
Wang P, Nair M S, Liu L, Iketani S, Luo Y, Guo Y, et al. “Antibody resistance of SARS-CoV-2 variants B.1.351 and B.1.1.7.” Nature. 2021; 593(7857): 130-135.
¹³⁹ Takeuchi T, Ishizuka M, Takayama H, Kureha K, Hamada M, and Umezawa H. “Pharmacology of Kasugamycin and the Effect on Pseudomonas Infection.” J Antibiot (Tokyo). 1965; 18: 107-110;
Umezawa H, Hamada M, Suhara Y, Hashimoto T, and Ikekawa T. “Kasugamycin, a new antibiotic.” Antimicrob Agents Chemother (Bethesda). 1965; 5: 753-757.
¹⁴⁰ USEPA. “Human Health Risk Assessment for Proposed Food Uses of the Fungicide Kasugamycin on Importing Fruiting Vegetables. Health Effects Division. 2005; Available from, as of Nov. 9, 2006: http://www.regulations.gov/fdmspublic/component/main.
¹⁴¹ Gopinath S, Kim M V, Rakib T, Wong P W, van Zandt M, Barry N A, et al. “Topical application of aminoglycoside antibiotics enhances host resistance to viral infections in a microbiota-independent manner.” Nature microbiology. 2018; 3(5):611-21.
¹⁴² Kamle S, Ma B, He C H, Akosman B, Zhou Y, Lee C M, et al. “Chitinase 3-like-1 is a Therapeutic Target That Mediates the Effects of Aging in COVID-19.” bioRxiv. 2021:2021.01.05.425478;
Lee J-H, Lee C-M, Kim M-O, Park J W, Kamle S, Akosman B, et al. “Kasugamycin is a novel chitinase 1 inhibitor with strong antifibrotic effects on pulmonary fibrosis.” bioRxiv. 2021:2021.02.25.432796.
¹⁴³ Takeuchi T, Ishizuka M, Takayama H, Kureha K, Hamada M, and Umezawa H. “Pharmacology of Kasugamycin and the Effect on Pseudomonas Infection.” J Antibiot (Tokyo). 1965; 18: 107-110; Ujváry I. In: Krieger R ed. “Hayes' Handbook of Pesticide Toxicology (Third Edition). New York: Academic Press; 2010: 119-229.
¹⁴⁴ Aleem A, Akbar Samad A B, and Slenker A K. “Emerging Variants of SARS-CoV-2 And Novel Therapeutics Against Coronavirus (COVID-19), StatPearls.” Treasure Island (FL):StatPearls Publishing LLC; 2021.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of preventing or treating an infection induced by a virus that enters cells via the cellular receptor Angiotensin-Converting Enzyme 2 (ACE2), comprising administering a therapeutically effective amount of a CHI3L1 inhibitor to a subject at risk of, or afflicted with, the infection.

2. The method of claim 1, wherein the virus is a coronavirus.

3. The method of claim 1, wherein the inhibitor of CHI3L1 is an antibody, antibody reagent, antigen-binding fragment thereof, or chimeric antigen receptor (CAR), that specifically binds a CHI3L1 polypeptide.

4. The method of claim 3, wherein the antibody, antibody reagent, antigen-binding portion thereof, or CAR comprising the complementarity determining regions (CDRs) of: (a) a light chain CDR1 having the amino acid sequence of SEQ ID NO: 4; (b) a light chain CDR2 having the amino acid sequence of SEQ ID NO: 5; (c) a light chain CDR3 having the amino acid sequence of SEQ ID NO: 6; (d) a heavy chain CDR1 having the amino acid sequence of SEQ ID NO: 1; (e) a heavy chain CDR2 having the amino acid sequence of SEQ ID NO: 2; and (f) a heavy chain CDR3 having the amino acid sequence of SEQ ID NO: 3.

5. The method of claim 4, wherein the antibody, antibody reagent, antigen-binding portion thereof, or CAR comprises a heavy chain sequence having the amino acid sequence selected from any one of SEQ ID NOS: 15-26.

6. The method of claim 4, wherein the antibody, antibody reagent, antigen-binding portion thereof, or CAR comprises a light chain sequence having the amino acid sequence selected from any one of SEQ ID NOS: 27-34.

7. The method of claim 4, wherein the antibody, antibody reagent, antigen-binding portion thereof, or CAR comprises a heavy chain sequence having the amino acid sequence selected from any of SEQ ID NOS: 15-26 and a light chain sequence having the amino acid sequence selected from any one of SEQ ID NOS: 27-34.

8. The method of claim 4, wherein the antibody, antibody reagent, antigen-binding portion thereof, or CAR comprises a heavy chain sequence having the amino acid sequence of SEQ ID NO: 13.

9. The method of claim 4, wherein the antibody, antibody reagent, antigen-binding portion thereof, or CAR comprises a light chain sequence having the amino acid sequence of SEQ ID NO: 14.

10. The method of claim 4, wherein the antibody, antibody reagent, antigen-binding portion thereof, or CAR comprises a heavy chain sequence having the amino acid sequence of SEQ ID NO: 13 and a light chain sequence having the amino acid sequence of SEQ ID NO: 14.

11. The method of claim 3, wherein the antibody, antibody reagent, antigen-binding portion thereof, or CAR further comprises a conservative substitution relative to the heavy chain sequence or the light chain sequence, wherein the conservative substitution is in a sequence not comprised by a CDR.

12. The method of claim 3, wherein the antibody, antibody reagent, antigen-binding portion thereof, or CAR is fully humanized except for the CDR sequences.

13. The method of claim 3, wherein the antibody, antibody reagent, antigen-binding portion thereof, or CAR is selected from the group consisting of: an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a diabody, a multispecific antibody, a dual specific antibody, an anti-idiotypic antibody, and a bispecific antibody.

14. The method of claim 1, wherein the subject is further administered a therapeutically effective amount of one or more of:
(i) an inhibitor CHI3L1 and chitinase 1;
(ii) an inhibitor of CHI3L1 phosphorylation;
(iii) remdesivir;
(iv) dexamethasone;
(v) REGEN-COV-2;
(vi) Baricitinib;
(vii) Sotrovimab;
(viii) PAXLOVID™; or
(ix) molnupiravir.

15-35. (canceled)


SEQUENCE LISTING (62 sequences)

SEQ ID NO: 1 - protein, 8 aa, synthetic construct
GYTFTNYG

SEQ ID NO: 2 - protein, 8 aa, synthetic construct
INTYTGEP

SEQ ID NO: 3 - protein, 13 aa, synthetic construct
ARLGYGKFYVMDY

SEQ ID NO: 4 - protein, 11 aa, synthetic construct
QSLVHSNGNTY

SEQ ID NO: 5 - skipped

SEQ ID NO: 6 - protein, 9 aa, synthetic construct
SQSTHVTWT

SEQ ID NO: 7 - DNA, 24 bp, synthetic construct
  source 1..24: mol_type = other DNA
gggtataccttcacaaactatgga

SEQ ID NO: 8 - DNA, 24 bp, synthetic construct
  source 1..24: mol_type = other DNA
ataaatacctacactggagagcca

SEQ ID NO: 9 - DNA, 39 bp, synthetic construct
  source 1..39: mol_type = other DNA
gcaagattgggatatggtaaattctatgttatggactac

SEQ ID NO: 10 - DNA, 33 bp, synthetic construct
  source 1..33: mol_type = other DNA
cagagccttgtacacagtaatggaaacacctat

SEQ ID NO: 11 - skipped

SEQ ID NO: 12 - DNA, 27 bp, synthetic construct
  source 1..27: mol_type = other DNA
tctcaaagtacacatgttacgtggacg

SEQ ID NO: 13 - protein, 120 aa, synthetic construct
QIQLVQSGPELKKPGETVKISCKASGYTFTNYGMNWVKQAPGKGLKWMGWINTYTGEPTYADDFKGRFAFSLETSASTAYLQINNLRNEDMSTYFCARLGYGKFYVMDYWGQGTSVTVSS

SEQ ID NO: 14 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVTWTFGGGTKLEIK

SEQ ID NO: 15 - protein, 120 aa, synthetic construct
QIQLVQSGSELKKPGASVKISCKASGYTFTNYGMNWVRQAPGQGLEWMGWINTYTGEPTYADDFKGRFVFSLDTSVSTAYLQISSLKAEDTSVYFCARLGYGKFYVMDYWGQGTSVTVSS

SEQ ID NO: 16 - protein, 120 aa, synthetic construct
QIQLVQSGSELKKPGASVKISCKASGYTFTNYGMNWVRQAPGQGLEWMGWINTYTGEPTYAQGFTGRFVFSLDTSVSTAYLQISSLKAEDTSVYFCARLGYGKFYVMDYWGQGTSVTVSS

SEQ ID NO: 17 - protein, 120 aa, synthetic construct
QIQLVQSGPELKKPGASVKISCKASGYTFTNYGMNWVRQAPGQGLKWMGWINTYTGEPTYADDFTGRFVFSLDTSVSTAYLQISSLKAEDTSVYFCARLGYGKFYVMDYWGQGTSVTVSS

SEQ ID NO: 18 - protein, 120 aa, synthetic construct
QIQLVQSGSELKKPGASVKISCKASGYTFTNYGMNWVRQAPGQGLKWMGWINTYTGEPTYADDFKGRFVFSLDTSVSTAYLQISSLKAEDTSVYFCARLGYGKFYVMDYWGQGTSVTVSS

SEQ ID NO: 19 - protein, 120 aa, synthetic construct
QIQLVQSGPELKKPGASVKISCKASGYTFTNYGMNWVKQAPGQGLKWMGWINTYTGEPTYAQGFTGRFVFSLDTSVSTAYLQISSLKAEDTSTYFCARLGYGKFYVMDYWGQGTSVTVSS

SEQ ID NO: 20 - protein, 120 aa, synthetic construct
QIQLVQSGPELKKPGASVKISCKASGYTFTSYAMNWVKQAPGQGLKWMGWINTYTGEPTYADDFKGRFVFSLDTSVSTAYLQISSLKAEDTSVYFCARLGYGKFYVMDYWGQGTSVTVSS

SEQ ID NO: 21 - protein, 120 aa, synthetic construct
QIQLVQSGHEVKQPGASVKISCKASGYTFTNYGMNWVPQAPGQGLEWMGWINTYTGEPTYADDFKGRFVFSLDTSASTAYLQISSLKAEDMSMYFCARLGYGKFYVMDYWGQGTSVTVSS

SEQ ID NO: 22 - protein, 120 aa, synthetic construct
QIQLVQSGHEVKQPGASVKISCKASGYTFTNYGMNWVPQAPGQGLEWMGWINTYTGEPTYAQGFTGRFVFSLDTSASTAYLQISSLKAEDMSMYFCARLGYGKFYVMDYWGQGTSVTVSS

SEQ ID NO: 23 - protein, 120 aa, synthetic construct
QIQLVQSGHEVKQPGASVKISCKASGYTFTNYGMNWVKQAPGQGLKWMGWINTYTGEPTYADDFTGRFVFSLDTSASTAYLQISSLKAEDMSMYFCARLGYGKFYVMDYWGQGTSVTVSS

SEQ ID NO: 24 - protein, 120 aa, synthetic construct
QIQLVQSGPEVKQPGASVKISCKASGYTFTNYGMNWVPQAPGQGLKWMGWINTYTGEPTYADDFTGRFVFSLDTSASTAYLQISSLKAEDMSMYFCARLGYGKFYVMDYWGQGTSVTVSS

SEQ ID NO: 25 - protein, 120 aa, synthetic construct
QIQLVQSGPEVKQPGASVKISCKASGYTFTNYGMNWVKQAPGQGLKWMGWINTYTGEPTYAQGFTGRFVFSLDTSASTAYLQISSLKAEDMSTYFCARLGYGKFYVMDYWGQGTSVTVSS

SEQ ID NO: 26 - protein, 120 aa, synthetic construct
QIQLVQSGPEVKQPGASVKISCKASGYSFTTYGMNWVKQAPGQGLEWMGWINTYTGEPTYADDFKGRFVFSLDTSASTAYLQISSLKAEDMSTYFCARLGYGKFYVMDYWGQGTSVTVSS

SEQ ID NO: 27 - protein, 112 aa, synthetic construct
DVVMTQSPLSLPVTLGQPASISCRSSQSLVHSNGNTYLNWYQQRPGQSPRLLIYKVSNRFSGVPDRFSGSGSGTDFTLKISRVEAEDVGVYFCSQSTHVTWTFGGGTKLEIK

SEQ ID NO: 28 - protein, 112 aa, synthetic construct
DVVMTQSPLSLPVTLGQPASISCRSSQSLVHSDGNTYLHWYQQRPGQSPRLLIYKVSNRFSGVPDRFSGSGSGTDFTLKISRVEAEDVGVYFCSQSTHVTWTFGGGTKLEIK

SEQ ID NO: 29 - protein, 112 aa, synthetic construct
DVVMTQSPLSLPVTLGQPASISCRSSQSLVHSNGNTYLHWYQQRPGQSPRLLIYKVSNRDSGVPDRFSGSGSGTDFTLKISRVEAEDVGVYFCSQSTHVTWTFGGGTKLEIK

SEQ ID NO: 30 - protein, 112 aa, synthetic construct
DVVMTQSPLSLPVTLGQPASISCRSSQSLVHSNGNTYLHWFQQRPGQSPRLLIYKVSNRFSGVPDRFSGSGSGTDFTLKISRVEAEDVGVYFCSQSTHVTWTFGGGTKLEIK

SEQ ID NO: 31 - protein, 112 aa, synthetic construct
DIVMTQTPLSLSVTPGQPASISCKSSQSLVHSNGNTYLHWYLQKPGQSPQLLIYKVSNRFSGVPDRFSGSGSGTDFTLKISRVEAEDVGVYYCSQSTHVTWTFGGGTKLEIK

SEQ ID NO: 32 - protein, 112 aa, synthetic construct
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVTWTFGGGTKLEIK

SEQ ID NO: 33 - protein, 112 aa, synthetic construct
DVVMTQSPLSLPVTLGQPASISCRSSQSLVHSNGNTYLHWFLQRPGQSPRLLIYKVSNRFSGVPDRFSGSGSGTDFTLKISRVEAEDVGVYYCSQSTHVTWTFGGGTKLEIK

SEQ ID NO: 34 - protein, 112 aa, synthetic construct
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVTWTFGGGTKLEIK

SEQ ID NO: 35 - protein, 12 aa, synthetic construct
FRGQEDASPDRF

SEQ ID NO: 36 - protein, 12 aa, synthetic construct
RGATVHRILGQQ

SEQ ID NO: 37 - protein, 12 aa, synthetic construct
ASSETGVGAPIS

SEQ ID NO: 38 - protein, 12 aa, synthetic construct
IKEAQPGKKQLL

SEQ ID NO: 39 - protein, 12 aa, synthetic construct
SNDHIDTWEWND

SEQ ID NO: 40 - protein, 12 aa, synthetic construct
YPGRRDKQHFTT

SEQ ID NO: 41 - protein, 12 aa, synthetic construct
LRLGAPASKLVM

SEQ ID NO: 42 - protein, 12 aa, synthetic construct
PGIPGRFTKEAG

SEQ ID NO: 43 - protein, 12 aa, synthetic construct
GSQRFSKIASNT

SEQ ID NO: 44 - protein, 12 aa, synthetic construct
GKVTIDSSYDIA

SEQ ID NO: 45 - protein, 12 aa, synthetic construct
GMLNTLKNRNPN

SEQ ID NO: 46 - protein, 12 aa, synthetic construct
SNTQSRRTFIKS

SEQ ID NO: 47 - protein, 5 aa, synthetic construct
GGGGS

SEQ ID NO: 48 - protein, 6 aa, synthetic construct
HHHHHH

SEQ ID NO: 49 - DNA, 21 bp, synthetic construct
  source 1..21: mol_type = other DNA
cgaagccgaagacctgttcta

SEQ ID NO: 50 - DNA, 21 bp, synthetic construct
  source 1..21: mol_type = other DNA
caagtgctccaactctgggat

SEQ ID NO: 51 - DNA, 21 bp, synthetic construct
  source 1..21: mol_type = other DNA
cttttgcctgggaattgcctc

SEQ ID NO: 52 - DNA, 21 bp, synthetic construct
  source 1..21: mol_type = other DNA
cctggttgctatgggtggtag

SEQ ID NO: 53 - DNA, 21 bp, synthetic construct
  source 1..21: mol_type = other DNA
tccagactccgatcatcaagc

SEQ ID NO: 54 - DNA, 23 bp, synthetic construct
  source 1..23: mol_type = other DNA
atcaaacctttagtgcagagtgg

SEQ ID NO: 55 - DNA, 21 bp, synthetic construct
  source 1..21: mol_type = other DNA
cagtctgagcacatctgtcct

SEQ ID NO: 56 - DNA, 20 bp, synthetic construct
  source 1..20: mol_type = other DNA
gggcaagtgtggactgttcc

SEQ ID NO: 57 - DNA, 21 bp, synthetic construct
  source 1..21: mol_type = other DNA
aacacaccgattctcgtcctc

SEQ ID NO: 58 - DNA, 19 bp, synthetic construct
  source 1..19: mol_type = other DNA
catcgccttccacttggtc

SEQ ID NO: 59 - DNA, 22 bp, synthetic construct
  source 1..22: mol_type = other DNA
aagtggtaatagtccccgaaga

SEQ ID NO: 60 - DNA, 23 bp, synthetic construct
  source 1..23: mol_type = other DNA
gctcatggtgttcagaattgtgt

SEQ ID NO: 61 - DNA, 21 bp, synthetic construct
  source 1..21: mol_type = other DNA
ctgtattccccgttgtgtagc

SEQ ID NO: 62 - DNA, 19 bp, synthetic construct
  source 1..19: mol_type = other DNA
ctcggagcatactgaggca